(19) United States
(12) Patent Application Publication
(10) Pub. No.: US 2025/0146083 A1
(43) Pub. Date: May 8, 2025

(54) IDENTIFYING FIBROBLAST SUBTYPES IN THYROID CANCER

(71) Applicant: Vanderbilt University, Nashville, TN (US)

(72) Inventors: Vivian L. Weiss, Nashville, TN (US); Matthew Loberg, Nashville, TN (US); George Xu, Nashville, TN (US)

(21) Appl. No.: 18/942,140

(22) Filed: Nov. 8, 2024

Related U.S. Application Data
(60) Provisional application No. 63/597,074, filed on Nov. 8, 2023.

(51) Int. Cl. C12Q 1/6886

(52) U.S. Cl. CPC C12Q 1/6886; C12Q 2600/112; C12Q 2600/158

(57) ABSTRACT

Identifying a fibroblast subtype in a thyroid cancer involves obtaining a fine needle aspiration (FNA) biopsy sample from a subject, the sample including nucleic acids; assaying the nucleic acids in the sample for an increase in the amount of mRNA produced by genes of a fibroblast gene signature relative to a control level of mRNA produced by the genes in a sample obtained from a healthy subject; and identifying the fibroblast subtype if there is an increase in the amount of mRNA in the sample relative to the control level.

### RELATED APPLICATIONS

This application claims priority from U.S. Provisional Application Ser. No. 63/597,074 filed Nov. 8, 2023, the entire disclosure of which is incorporated herein by this reference.

### GOVERNMENT INTEREST

This invention was made with government support under grant numbers CA272875 and CA240901 awarded by the National Institutes of Health. The government has certain rights in the invention.

### INTRODUCTION

Thyroid cancer is common, with both incidence and mortality rates increasing.^{1,2} Most malignant thyroid lesions are indolent, well-differentiated tumors such as papillary or follicular thyroid carcinomas (PTC and FTC, respectively). These tumors can be successfully treated with surgical resection of the thyroid followed by radioactive iodine.^{3,4} However, many thyroid cancers recur or progress after such standard-of-care treatment. Rarely, recurrence can even occur decades after initial therapy and may involve distant metastasis or transformation to poorly differentiated (PDTC) or anaplastic thyroid cancer (ATC).⁵ ATC is one of the most lethal cancers in existence. The median survival of patients with ATC is just 3-5 months—indicating a dire need to better understand drivers of thyroid cancer progression.⁶

DNA and RNA sequencing have revolutionized our understanding of many tumors, providing insight into underlying biology and drivers of aggressive disease. However, the genomic understanding of thyroid cancer has lagged behind other tumors. Until recently, thyroid cancer molecular testing was used almost exclusively for diagnostic purposes at the time of initial biopsy.^{3,7} Genomic studies have identified common driver mutations in well-differentiated thyroid cancers, allowing commercial genomic classifiers to distinguish malignant from benign lesions with high sensitivity, specificity, and accuracy.^{8,9,10,11,12,13} BRAF V600E and RAS mutations are mutually exclusive and represent the most common driver alterations in well-differentiated thyroid cancers. The frequency of these alterations has led to the molecular classification of thyroid cancers as either BRAF-like or RAS-like, based on the gene expression patterns of BRAF V600E and RAS-mutant PTCs.¹⁴ Other alterations detected by diagnostic molecular tests include gene fusions such as NTRK1/3, PAX8/PPARG, and RET; copy number alterations; microRNA dysregulation; and gene expression abnormalities.^{15,16,17}

Despite these advances in molecular diagnostics, thyroid cancer prognostication and post-surgical treatment are still largely guided by clinical and histopathologic features. This cancer management is in stark contrast to the biomarker-driven personalized management of many other cancers, such as non-small cell lung cancer.^{18,19} The lack of molecular biomarker testing for thyroid cancer is due to our limited understanding of the drivers of advanced disease. While patients with BRAF-like tumors have slightly worse outcomes (5% mortality in BRAF-mutant PTC versus 1% in RAS-mutant PTC),²⁰ the majority of BRAF-like thyroid cancers have an excellent prognosis. Recent large sequencing studies have implicated TP53, PIK3CA, and TERT promoter (TERTp) mutations in aggressive thyroid cancer, particularly in combination with BRAF V600E.^{21,22,23,24,25,26} This work has led to the development of the first commercial molecular-based test for high-risk disease.²⁷ Despite this tremendous advance, there are still patients with recurrent, metastatic, and de-differentiated thyroid cancer that lack high-risk mutations. Additional tools are needed to further enhance the risk-stratification and management of patients.

The role of the tumor microenvironment in cancer progression is an active area of investigation and has led to many advances in prognostication and therapy.²⁸ However, there has been limited research in the microenvironment of thyroid cancer. To this end, recent work identified a subgroup of BRAF-like lesions enriched in cancer-associated fibroblasts (CAFs) that may have more aggressive behavior.²⁹ Another study showed that in thyroid tumors driven by BRAF V600E mutation and PTEN loss, fibroblasts may promote progression by remodeling collagen in the tumor microenvironment.³⁰ Other research has suggested roles of infiltrating immune cells such as macrophages in supporting aggressive thyroid cancer behaviors.^{31,32} As such, clinical trials are ongoing to evaluate the efficacy of checkpoint inhibitor therapy for anaplastic thyroid carcinoma.^{33,34,35,36,37,38}

Thus, obtaining biopsy samples that contain CAFs is useful for studying the tumor microenvironment and understanding the role of these cells in cancer progression. Surgical biopsy is one technique that is used to obtain samples including CAFs, which involves the surgical removal of a portion of the tumor or the entire tumor, either through open biopsy or less invasive techniques like laparoscopy. This method yields large and comprehensive tissue samples, allowing for a detailed analysis of the tumor microenvironment, including CAFs. The main disadvantage is that surgical biopsies are more invasive, requiring longer recovery times and carrying higher risks of complications compared to needle biopsies.

Core needle biopsy (CNB) is another method used to obtain samples containing CAFs, which makes use of a large needle to extract a core of tissue, often guided by imaging techniques such as ultrasound, CT, or MRI. This method provides large tissue samples, which are likely to contain a representative mix of tumor cells and stromal components, including CAFs. However, CNB can be invasive, causing discomfort and complications such as bleeding or infection.

Accordingly, there remains a need in the art for improved methods for obtaining and analyzing samples containing cancer-associated fibroblasts.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

Embodiments of the presently-disclosed subject matter include a method for identifying enrichment of a fibroblast subtype in a thyroid cancer, which comprises obtaining biopsy sample from a subject, the sample including nucleic acids; assaying the nucleic acids in the sample for an increase in the amount of mRNA produced by genes consisting of 3-80 of the genes set forth in Table 1A, Table 1B, Table 1C, Table 1D, Table 1E, Table 2A, Table 2B, Table 2C, Table 2D, Table 2E, Table 2F, Table 3, Table 4, and Table 5 relative to a control level of mRNA produced by the genes in a sample obtained from a healthy subject; and identifying an enrichment of the fibroblast subset population if there is an increase in the amount of mRNA in the sample relative to the control level.

Embodiments of the presently-disclosed subject matter include a method for identifying a fibroblast subtype, macrophage subtype, or myofibroblast subtypes in a thyroid cancer, which comprise obtaining biopsy sample from a subject, the sample including nucleic acids; assaying the nucleic acids in the sample for an increase in the amount of mRNA produced by genes consisting of 3-80 of the genes set forth in Table 1A, Table 1B, Table 1C, Table 1D, Table 1E, Table 2A, Table 2B, Table 2C, Table 2D, Table 2E, Table 2F, Table 3, Table 4, and Table 5 relative to a control level of mRNA produced by the genes in a sample obtained from a healthy subject; and identifying an enrichment of fibroblast subtype, macrophage subtype, or myofibroblast subtypes if there is an increase in the amount of mRNA in the sample relative to the control level.

Embodiments of the presently-disclosed subject matter include a method of detecting expression of genes in a fine needle aspiration (FNA) biopsy sample from a subject, which comprises the steps of determining expression levels in the FNA biopsy sample of each of the gene of a fibroblast gene signature, wherein the fibroblast gene signature comprises 3-80 of the genes set forth in Table 1A, Table 1B, Table 1C, Table 1D, Table 1E, Table 2A, Table 2B, Table 2C, Table 2D, Table 2E, Table 2F, Table 3, Table 4, and Table 5; wherein the expression levels of each of the genes are determined by sequencing genetic material in the FNA biopsy sample.

Embodiments of the presently-disclosed subject matter include a treatment method, which comprises determining expression levels in a fine needle aspiration (FNA) biopsy sample from a subject of each gene of a fibroblast gene signature, wherein the fibroblast gene signature comprises 3-80 of the genes set forth in Table 1A, Table 1B, Table 1C, Table 1D, Table 1E, Table 2A, Table 2B, Table 2C, Table 2D, Table 2E, Table 2F, Table 3, Table 4, and Table 5; wherein the expression levels of each of the genes are determined by sequencing genetic material in the FNA biopsy sample; comparing the expression levels to expression levels for each gene in a control sample from a normal healthy individual or a benign individual; and administering radioactive iodine to the subject when there is overexpression of the genes in the FNA biopsy sample relative expression levels of the genes in a control sample from a normal healthy individual or a benign individual.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A-1F. Mutations associated with aggressive thyroid cancer. (FIG. 1A) Cohort summary quantifying number of samples within each diagnosis. Abbreviations: MNG, multinodular goiter; HT, Hashimoto thyroiditis; FA, follicular adenoma; OA, oncocytic adenoma; NIFTP, noninvasive follicular thyroid neoplasm with papillary-like nuclear features; EFVPTC, encapsulated follicular variant papillary thyroid carcinoma, OTC, oncocytic thyroid carcinoma; FTC, follicular thyroid carcinoma; PTC, papillary thyroid carcinoma; IFVPTC, infiltrative follicular variant papillary thyroid carcinoma, PDTC, poorly differentiated thyroid carcinoma; ATC, anaplastic thyroid carcinoma. (FIG. 1B) Schematic representation showing patient disease categorization as well as pipeline used for sequencing data collection. 312 formalin-fixed, paraffin-embedded (FFPE) resection samples underwent high-throughput sequencing. (FIG. 1C) Oncoplot showing mutational landscape of malignant thyroid lesions. The 20 most frequently mutated genes after filtering are displayed. Annotation bars above show diagnosis, tissue location of the lesion sequenced, sex of the patient, age of the patient at surgery, and patient disease categorization. Detected thyroid cancer fusions are also shown. (FIG. 1D-1F) Progression-free survival (PFS) plots for patients with malignant thyroid lesions with and without TERT promoter mutation (FIG. 1D), TP53 mutation (FIG. 1E), and PIK3CA mutation (FIG. 1F). p values were calculated with log rank test.

FIG. 2A-2C. Association of TERT promoter (TERTp), TP53, and PIK3CA mutations and aggressive disease. (FIG. 2A) Overall survival plots for patients with malignant thyroid lesions with and without TERTp, TP53, and PIK3CA mutations. P values were calculated with log-rank test. (FIG. 2B) Indolent vs aggressive samples, top 20 mutations after mutation filtering in local PTC, IFVPTC, and EFVPTC lesions. Abbreviations: PTC=Papillary Thyroid Carcinoma; IFVPTC=Infiltrative Follicular Variant Papillary Thyroid Carcinoma; EFVPTC=Encapsulated Follicular Variant Papillary Thyroid Carcinoma. (FIG. 2C) Indolent vs aggressive samples, top 20 mutations after mutation filtering in local FTC and OTC lesions. Abbreviations: FTC=Follicular Thyroid Carcinoma; OTC=Oncocytic Thyroid Carcinoma. Red asterisks indicate TERTp, TP53, and PIK3CA mutations.

FIG. 3A-3H. Molecular Aggression and Prediction (MAP) score. (FIG. 3A) Diagram outlining BRAF-RAS score (BRS) classification method. Positive BRS lesions were categorized as RAS-like, and negative BRS lesions were classified as BRAF-like. (FIG. 3B) Boxplots showing BRS from local disease samples. Shading indicates clinical behavior (pink, aggressive; black, indolent; gray, no clinical follow-up after sample collection). Abbreviations: FA, follicular adenoma; OA, oncocytic adenoma; FTC, follicular thyroid carcinoma; OTC, oncocytic thyroid carcinoma; EFVPTC, encapsulated follicular variant papillary thyroid carcinoma, NIFTP, noninvasive follicular thyroid neoplasm with papillary-like nuclear features; PTC, papillary thyroid carcinoma; IFVPTC, infiltrative follicular variant papillary thyroid carcinoma, PDTC, poorly differentiated thyroid carcinoma; ATC, anaplastic thyroid carcinoma. (FIG. 3C) Diagram outlining method for identifying genes enriched in samples from patients with aggressive vs. indolent disease. (FIG. 3D) Venn diagrams showing the overlap of genes that are upregulated in BRAF-like lesions, RAS-like lesions, and aggressive disease lesions. Thresholds for upregulation was an adjusted p value<0.05 and fold change of ≥4 (aggressive disease upregulated for BRAF-like Venn diagram) or ≥2 (BRAF-like upregulated, RAS-like upregulated, and aggressive disease upregulated for RAS-like Venn diagram). (FIG. 3E) Boxplots of MAP score calculated from the 549 genes that overlap between BRAF-like and aggressive lesions in (FIG. 3C) (FTC, follicular thyroid carcinoma; OTC, oncocytic thyroid carcinoma; EFVPTC, encapsulated follicular variant papillary thyroid carcinoma, NIFTP, noninvasive follicular thyroid neoplasm with papillary-like nuclear features; PTC, papillary thyroid carcinoma; IFVPTC, infiltrative follicular variant papillary thyroid carcinoma, PDTC, poorly differentiated thyroid carcinoma; ATC, anaplastic thyroid carcinoma). Dots indicate lesions from patients with aggressive disease. (FIG. 3F) Boxplots of MAP score in TCGA samples plotted by histology, extrathyroidal extension, and disease stage. Three outliers for MAP score were omitted for improved visualization of plots. p values calculated with Kruskal-Wallis test with pairwise Wilcoxon rank-sum test and Bonferroni's correction. (FIG. 3G) Gene ontology results for the 549 genes comprising MAP score, showing enrichment of extracellular matrix, immune, cell cycle, and epithelial differentiation processes. Statistical analysis of fold enrichment was performed with Fisher's exact with false discovery rate correction. (FIG. 3H) Summary diagram of MAP score components predicted to be enriched in gene ontology analysis.

FIG. 4A-4C. BRAF-RAS Score (BRS) is not correlated with progression-free survival (PFS) in differentiated thyroid cancer. (FIG. 4A) Normalized gene expression heatmap showing the 69 genes used to calculate BRS. Samples are ordered by diagnosis, and genes are ordered by hierarchical clustering. Diagnoses abbreviations: FA=Follicular Adenoma; OA=Oncocytic Adenoma; NIFTP=Noninvasive Follicular Thyroid Neoplasm with Papillary-like Nuclear Features; EFVPTC=Encapsulated Follicular Variant Papillary Thyroid Carcinoma; OTC=Oncocytic Thyroid Carcinoma; FTC=Follicular Thyroid Carcinoma; PTC=Papillary Thyroid Carcinoma; IFVPTC=Infiltrative Follicular Variant Papillary Thyroid Carcinoma; PDTC=Poorly Differentiated Thyroid Carcinoma; ATC=Anaplastic Thyroid Carcinoma. (FIG. 4B) PFS of BRAF-like (red) and RAS-like (blue) differentiated thyroid tumors (local disease location only, lesion subtypes FTC, OTC, EFVPTC, NIFTP, PTC, IFVPTC, IFVPTC) after initial treatment. P values were calculated with log-rank test. (FIG. 4C) PFS plot of patients with differentiated and transformed thyroid lesions (local disease location only, all lesion subtypes except MNG, OTC, FA, and OA) with BRAF-like (red) vs RAS-like (blue). P values were calculated with log-rank test.

FIG. 5A-5H. TDS, PI3K and ERK scores in thyroid carcinoma. (FIG. 5A) Normalized gene expression heatmap showing genes used to calculate PI3K-AKT-mTOR score (PI3K score). Samples are ordered by diagnosis, and genes are ordered by hierarchical clustering. (FIG. 5B) Box plot of PI3K score by diagnosis. (FIG. 5C) Normalized gene expression heatmap showing the 52 genes used to calculate ERK gene expression score. Samples are ordered by diagnosis, and genes are ordered by hierarchical clustering. (FIG. 5D) Box plot of ERK score by diagnosis. (FIG. 5E) Normalized gene expression heatmap showing 16 genes used to calculate the Thyroid Differentiation score, TDS. PTC samples with background Hashimoto's thyroiditis (HT) excluded due to low tumor purity. Samples are ordered by diagnosis, and genes are ordered by hierarchical clustering. (FIG. 5F) Box plot of TDS score by diagnosis. PTC samples with background HT excluded due to low tumor purity. (FIG. 5G) Scatterplot with linear model of the relationship between BRS and TDS, neoplastic samples only (FA, OA, NIFTP, EFVPTC, OTC, FTC, PTC, IFVPTC, PDTC, ATC). PTC samples with background HT excluded due to low tumor purity. (FIG. 5H) Gene Ontology Analysis of thyroid processes upregulated in RAS-like tumors. Diagnosis abbreviations: FA=Follicular Adenoma; OA=Oncocytic Adenoma; NIFTP=Noninvasive Follicular Thyroid Neoplasm with Papillary-like Nuclear Features; EFVPTC=Encapsulated Follicular Variant Papillary Thyroid Carcinoma; OTC=Oncocytic Thyroid Carcinoma; FTC=Follicular Thyroid Carcinoma; PTC=Papillary Thyroid Carcinoma; IFVPTC=Infiltrative Follicular Variant Papillary Thyroid Carcinoma; PDTC=Poorly Differentiated Thyroid Carcinoma; ATC=Anaplastic Thyroid Carcinoma.

FIG. 6A-6H. MAP score is associated with CAF, neutrophil, and M2 macrophage infiltrate in thyroid tumors. (A) Volcano plot showing differentially expressed genes (fold change>2, adjusted p value<0.05) between malignant localized thyroid lesions with positive and negative MAP score. Samples with Hashimoto thyroiditis were excluded. Select markers of extracellular matrix, cancer-associated fibroblasts, and key immune cell populations are labeled. (FIGS. 6B and 6C) Boxplots of all malignant thyroid lesions, excluding samples with Hashimoto thyroiditis, showing log-transformed CAF markers FAP and LRRC15, M2 macrophage polarization markers MRC1 and CD163, and neutrophil markers ELANE and FCGR3B from bulk RNA sequencing data (FIG. 6B) or log-transformed EPIC CAF score, CIBERSORT absolute value M2 macrophage score, and TIMER neutrophils score (FIG. 6C). Samples are categorized as negative MAP score and positive MAP score. p values were calculated with Wilcoxon rank-sum test. (FIG. 6D) Heatmap of select deconvolution results from TCGA, an external well-differentiated thyroid cancer cohort. BRS, MAP score category, and MAP score annotations displayed on the top of the heatmap, followed by TIMER scores, CIBERSORT absolute value M1/M2 macrophage scores, and EPIC CAF scores. Samples are sorted by increasing MAP score from left to right. (FIG. 6E) Boxplots of EPIC CAF score, CIBERSORT absolute value M2 macrophage score, and TIMER neutrophil score, with samples organized into the following thyroid lesion subtype groups: RAS-like (FTC, OTC, EFVPTC, and NIFTP), BRAF-like (PTC and IFVPTC), PDTC, and ATC. All scores are on a log 2 scale. p values were calculated with Kruskal-Wallis test with pairwise Wilcoxon rank-sum test and Bonferroni's correction. (FIG. 6F) Clustering of transcriptomic data from a representative ATC spatial transcriptomics sample with spatial mapping of clusters (upper left), UMAP (lower left), and differential gene expression heatmap showing the top 10 markers for each of the clusters (right). Clusters are labeled as CAF, ATC tumor cells, and intermixed immune cells based on marker genes in the heatmap. (FIG. 6G) SpaCET spatial deconvolution showing estimated spatial capture area cell fractions for CAF and macrophage for eight ATC samples. (FIG. 6H) Boxplots of MAP scores in ATCs, split into groups with either low or high histologic quantification of CAFs, FAP+ CAFs, neutrophils, and MRC1+ macrophages. Representative histology of specific cell types is shown to the left of quantification. p values were calculated with Wilcoxon rank-sum test.

FIG. 7. CAFs, neutrophils, and macrophages are enriched in positive MAP score thyroid cancers. Heatmap of deconvolution results. Diagnosis, tissue location, aggressive disease, MAP score category, and MAP score annotations displayed on the top of the heatmap, followed by sample location, TIMER and absolute value CIBERSORT (CIBERSORT-Abs) immune deconvolution scores, EPIC and MCPCOUNTER CAF scores, and TIDE results including TIDE score, dysfunction score, and exclusion score. Samples are arranged by increasing MAP score from left to right within each diagnosis. PTC samples from patients with Hashimoto Thyroiditis (HT) were excluded.

FIG. 8A-8C. Immune cell infiltrate is associated with MAP score in external well-differentiated thyroid cancer cohort. (FIG. 8A) Heatmap of immune deconvolution results from TCGA thyroid cohort bulk RNA-sequencing data. Histological type, BRAF mutation, RAS mutation status, BRS, MAP score category, and MAP score annotations displayed on the top of the heatmap, followed by TIMER and absolute value CIBERSORT immune deconvolution scores, EPIC and MCPCOUNTER CAF scores, and TIDE results including TIDE score, dysfunction score, and exclusion score. Samples are arranged by increasing MAP score from left to right. (FIG. 8B) Box plots showing statistical comparison between negative and positive MAP score PTCs (TCGA cohort) for select populations from the deconvolution heatmap in A. P values were calculated with Wilcoxon rank-sum test. (FIG. 8C) Box plots showing statistical comparison between negative and positive MAP score PTCs (TCGA cohort) for marker genes of M2 macrophages, CAFs, and neutrophils. P values were calculated with Wilcoxon rank-sum test.

FIG. 9A-9H. MAP score is associated with tumor microenvironment composition in ATCs. (FIG. 9A) Heatmap of select deconvolution results for PDTCs and ATCs. Diagnosis, tissue location, aggressive disease, MAP score category, and MAP score annotations displayed on the top of the heatmap, followed by TIMER scores, M1/M2 absolute value CIBERSORT immune deconvolution scores, and EPIC CAF scores. Samples are arranged by increasing MAP score from left to right within each diagnosis. Representative histology shown for PDTC, lymphocyte-rich ATC, and CAF-rich ATC. (FIG. 9B) Moderate and high MAP score ATC tumor categorization diagram (tumors split by 50th percentile MAP score), and boxplots of EPIC CAF score, CIBERSORT absolute M2 macrophage score, CIBERSORT absolute M1 macrophage score, and TIMER CD8+ T cell score, comparing moderate and high MAP score ATCs. All scores are on a log 2 scale. p values were calculated with Wilcoxon rank-sum test. (FIG. 9C) Representative multiplex IF image of ATC with MRC1+ macrophages and adjacent FAP+fibroblasts. White arrows indicate MRC1+ cells. Green, pan-cytokeratin, white, MRC1, FAP, nuclear. Quantification of staining below showing the relationship between FAP staining and MRC1 staining. R2 and p value generated from a linear model with FAP staining score as the independent variable. (FIG. 9D) Linear model of M2 macrophage and fibroblast co-localization from SpaCET deconvolution of eight ATCs as a dependent variable of MAP score (left), with representative spatial capture area M2 macrophage and fibroblast non-parametric rho correlation plots of moderate and high MAP score ATCs (right). (FIG. 9E) Representative images of lymphocyte deconvolution from spatial transcriptomics data of moderate and high MAP score tumors (left) and comparison of average lymphoid spatial capture fraction between moderate and high MAP score tumors for all eight spatial transcriptomic samples (right). p value was calculated with Wilcoxon rank-sum test. (FIG. 9F) Representative CD3 stained samples showing histologically excluded or included T cells. (FIG. 9G) TIDE exclusion score in moderate and high MAP score ATCs and association with CD3 staining. p values were calculated with Wilcoxon rank-sum test. (FIG. 9H) TIDE score in moderate and high MAP score tumors. p value was calculated with Wilcoxon rank-sum test.

FIG. 10A-10C. Molecular Aggression and Prediction (MAP) score subdivides the immune infiltrate in anaplastic thyroid carcinoma. (FIG. 10A-10C) Box plots showing statistical comparison between anaplastic thyroid carcinomas with moderate (<50^{th} percentile) and high (>50^{th} percentile) MAP scores for bulk RNA-sequencing marker genes of cancer-associated fibroblasts (FIG. 10A), M2 macrophages (FIG. 10B), and cytotoxic T-cells (FIG. 10C). P values were calculated with Wilcoxon rank-sum test.

FIG. 11A-11C. Spatial transcriptomics highlights the distinct tumor microenvironments of MAP-high and MAP-moderate ATCs. (FIG. 11A) Spatial capture area non-parametric Spearman correlation plots for percent of individual spots that are predicted cancer-associated fibroblast (x-axis) and M2 macrophage (y-axis) by SpaCET deconvolution of anaplastic thyroid carcinoma (ATC) spatial transcriptomic data (top row=Moderate MAP score samples; bottom row=high MAP score samples). (FIG. 11B) Hematoxylin and eosin staining of ATC spatial transcriptomic samples (top row=moderate MAP score samples; bottom row=high MAP score samples). Three samples were only stained with hematoxylin due to supply chain shortages. (FIG. 11C) Spatial depiction of SpaCET lymphoid capture area deconvolution (MAP score moderate samples on the left, MAP score high samples on the right). Box plot depicts quantitative assessment of average capture area lymphoid deconvolution for each sample and compares MAP moderate ATCs to MAP high ATCs. P value was calculated with Wilcoxon rank-sum test.

FIG. 12A-12D. MAP score is associated with disease progression and predicted response to immune checkpoint blockade therapy. (FIG. 12A) Diagram showing 5-year survival of patients with well-differentiated thyroid cancer and patients with transformed thyroid cancer. Table showing percent of samples that are metastatic in the internal cohort from Vanderbilt University Medical Center and University of Washington Medical Center and the external cohort from TCGA. (FIG. 12B) PFS in patients with well-differentiated and transformed thyroid cancer (left), as well as patients with only well-differentiated thyroid cancer (right), with positive or negative MAP score. p values were calculated with log rank test. (FIG. 12C) Disease-free survival in TCGA patients with well-differentiated thyroid cancer with positive or negative MAP score. p values were calculated with log rank test. (FIG. 12D) Receiver operating characteristic curve showing association between aggression and TERTp/TP53/PIK3CA mutation, MAP score, and TERTp/TP53/PIK3CA mutation+MAP score, for patients with well-differentiated and transformed thyroid cancer (left), well-differentiated thyroid cancer (center-left), well-differentiated thyroid cancer sampled prior to aggression (center-right), and well-differentiated thyroid cancer sampled prior to aggression excluding any samples with a mutation in TERTp, TP53, and PIK3CA (right). Area under the curve values with 95% confidence intervals are shown. Metastatic tumors were excluded.

FIG. 13A-13C. Molecular Aggression and Prediction Score (MAP) is associated with disease progression. (FIG. 13A and FIG. 13B), Overall survival (OS) with positive or negative MAP score for patients with differentiated and transformed lesions (local disease location only) (FIG. 13A) and for TCGA patients (FIG. 13B). P values were calculated with log-rank test. (FIG. 13C), Forest plots of logistic regression results. Odds ratios shown for TERTp/TP53/PIK3CA mutation, and odds ratio per interquartile range increase shown for all other variables. 95% confidence interval indicated by colored lines.

FIG. 14. Outcome and therapy prediction using MAP score. Summary diagram showing the otential role of MAP score for risk strategifying thyroid tumors.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes a method of detecting expression of genes in a fine needle aspiration (FNA) biopsy sample from a subject. The presently-disclosed subject matter also includes method for identifying enrichment of a fibroblast subtype in a sample from a subject. The presently-disclosed subject matter also includes a method for identifying fibroblast subtypes in a sample from a subject. The presently-disclosed subject matter also includes a method for identifying myofibroblast subtypes in a sample from a subject. The presently-disclosed subject matter also includes a method for identifying macrophage subtypes in a sample from a subject.

In the context of cancer, fibroblasts, myofibroblasts, and macrophages play complex roles in the tumor microenvironment. Their subtypes vary by function, phenotype, and contribution to cancer progression, particularly in cancers like thyroid cancer.

In cancers, cancer-associated fibroblasts (CAFs) have diverse roles in supporting tumor growth, immune evasion, and extracellular matrix (ECM) remodeling. There are a variety of cancer-associated fibroblast (CAF) subtypes, each playing distinct roles in tumor progression and metastasis. SFRP2+ fibroblasts, which express Secreted Frizzled-Related Protein 2 (SFRP2), are involved in modulating Wnt signaling pathways and are known to promote tumor growth and angiogenesis, contributing to the aggressive behavior of thyroid cancer. Another subtype, CD36+ fibroblasts, identified through single-cell RNA sequencing, express CD36, a receptor involved in fatty acid metabolism, and significantly promote the proliferation, migration, and invasion of PTC cells while inhibiting their apoptosis. Myofibroblast CAFs (myCAFs) are typically located near tumor cells and express high levels of α-smooth muscle actin (α-SMA). These fibroblasts are involved in extracellular matrix (ECM) remodeling and provide structural support to the tumor, facilitating cancer cell invasion and metastasis. Inflammatory CAFs (iCAFs), positioned further away from tumor cells, express lower levels of α-SMA but secrete higher levels of inflammatory cytokines such as IL-6, IL-8, and IL-11, contributing to a pro-inflammatory tumor microenvironment that supports tumor growth and immune evasion. Antigen-presenting CAFs (apCAFs) have the ability to present antigens and express molecules involved in immune modulation, influencing the immune response within the tumor microenvironment and potentially affecting the efficacy of immunotherapies. These fibroblast subtypes highlight the complexity of the tumor microenvironment in thyroid cancer and underscore the diverse roles that CAFs play in cancer progression. Secretory CAFs (sCAFs) secrete growth factors (e.g., TGF-β) and ECM components, facilitating tumor growth and metastasis.

Myofibroblasts are activated fibroblasts with a contractile phenotype, often arising in response to tissue injury but also present in cancer. In cancer, they are known to support tumor growth and metastasis by remodeling ECM and secreting pro-tumorigenic factors. There are a variate of myofibroblast subtypes. α-SMA-expressing myofibroblasts are characterized by high levels of α-SMA and a contractile phenotype, and which promote ECM stiffness and remodeling. Fibroblast-activation-protein-alpha (FAPα)-positive myofibroblasts promote tumor invasion and immunosuppression within the tumor microenvironment. TGF-β-induced myofibroblasts arise in response to TGF-β signaling, enhancing ECM production and contributing to tissue fibrosis and immune modulation. In thyroid cancer, these cells play a role in creating a pro-fibrotic environment that can support cancer progression and resistance to therapy.

Macrophages in the tumor microenvironment, often called tumor-associated macrophages (TAMs), can adopt various phenotypes that support or suppress tumor growth. There are a number of TAM subtypes. M1-like macrophages (pro-inflammatory) are classically activated macrophages with anti-tumor functions, producing pro-inflammatory cytokines (e.g., TNF-α, IL-1β) and promoting immune responses. M2-like Macrophages (pro-tumor) are alternatively activated macrophages associated with tissue repair, immunosuppression, and tumor progression. They secrete anti-inflammatory cytokines (e.g., IL-10, TGF-β) and support angiogenesis and metastasis. M2a, M2b, M2c Subtypes are further differentiations within M2 macrophages: M2a is induced by IL-4/IL-13, associated with tissue repair; M2b is induced by immune complexes, linked to immunoregulation, and M2c is induced by IL-10, involved in matrix remodeling and immunosuppression. Metastasis-associated macrophages are specialized TAMs located in areas of metastatic growth, facilitating cancer cell survival and colonization. In thyroid cancer, TAMs are often skewed towards the M2 phenotype, which can contribute to a tumor-friendly environment by suppressing immune responses and supporting tumor growth and invasion.

Methods of the presently-disclosed subject matter identifying enrichment of a fibroblast subtype, identifying fibroblast subtypes, identifying myofibroblast subtypes, and/or identifying macrophage subtypes in a sample from a subject. Involve obtaining a biopsy sample from a subject.

As compared to other biopsy techniques, such as surgical biopsy and core needle biopsy, fine needle aspiration (FNA) biopsy is highly useful and desirable for several reasons. FNA is minimally invasive, utilizing a thin needle to extract cells or fluid from a target area, which results in less discomfort, minimal scarring, and a quicker recovery time compared to other biopsy methods like core needle or surgical biopsies. The procedure is also quick and efficient, often taking just a few minutes and can be performed in a healthcare provider's office, making it convenient for both patients and healthcare providers. With the aid of imaging techniques like ultrasound or CT scans, FNA can target deep or hard-to-reach areas, ensuring accurate sampling. Moreover, the risk of complications, such as infection or bleeding, is relatively low with FNA, making it a safer option for many patients.

Fine needle aspiration (FNA) biopsy samples are generally useful for diagnosing various conditions due to their minimally invasive nature and efficiency. However, when it comes to evaluating fibrotic lesions or fibroblasts, FNA has long been considered to be ineffective.^{89, 90, 91} For instance, tumors with abundant stromal fibrosis have lower diagnostic yields with FNA. The dense fibrous tissue can hinder the needle from obtaining sufficient cellular material, leading to non-diagnostic or inconclusive results. Additionally, the smaller sample size obtained through FNA can be a limitation, particularly for lesions with heterogeneous components or those requiring extensive histologic architecture for accurate diagnosis. This has especially been the believe in connection with fibrotic lesions. There is a long-standing believe that fibrotic lesions (i.e., full of fibroblasts) fail to adequately aspirate on FNA.⁸⁹ FNA has consistently been reported to yield non-diagnostic samples,⁸⁹⁻⁹¹ such that FNA has been considered an ineffective tool for obtaining diagnostic samples including fibroblasts, including cancer associated CAFs. Accordingly, the skilled artisan would recognize FNA is a valuable tool, but would avoid it for evaluating fibrotic lesions or fibroblasts due to the challenges in obtaining adequate and representative samples, selecting instead methods such as core needle biopsy or surgical biopsy.

Accordingly, it is unexpected and surprising that the inventors discovered that the methods disclosed herein could be used with FNA biopsy samples to effectively identify enrichment of a fibroblast subtype, fibroblast subtypes, myofibroblast subtypes, and/or macrophage subtype.

Embodiments of the presently-disclosed subject matter include a method for identifying enrichment of a fibroblast subtype in a thyroid cancer, which comprises obtaining biopsy sample from a subject, the sample including nucleic acids; assaying the nucleic acids in the sample for an increase in the amount of mRNA produced by genes consisting of 3-80 of the genes set forth in Table 1A, Table 1B, Table 1C, Table 1D, Table 1E, Table 2A, Table 2B, Table 2C, Table 2D, Table 2E, Table 2F, Table 3, Table 4, and Table 5 relative to a control level of mRNA produced by the genes in a sample obtained from a healthy subject; and identifying an enrichment of the fibroblast subset population if there is an increase in the amount of mRNA in the sample relative to the control level. In some embodiments, the biopsy sample is a fine needle aspiration (FNA) biopsy sample. In some embodiments, a portion of the genetic material in the FNA biopsy sample is from fibroblasts. In some embodiments, the subject is being evaluated for presence, metastasis, and/or recurrence of thyroid cancer. In some embodiments, the biopsy sample is taken from a tumor microenvironment.

Embodiments of the presently-disclosed subject matter include a method for identifying a fibroblast subtype, macrophage subtype, or myofibroblast subtypes in a thyroid cancer, which comprise obtaining biopsy sample from a subject, the sample including nucleic acids; assaying the nucleic acids in the sample for an increase in the amount of mRNA produced by genes consisting of 3-80 of the genes set forth in Table 1A, Table 1B, Table 1C, Table 1D, Table 1E, Table 2A, Table 2B, Table 2C, Table 2D, Table 2E, Table 2F, Table 3, Table 4, and Table 5 relative to a control level of mRNA produced by the genes in a sample obtained from a healthy subject; and identifying an enrichment of fibroblast subtype, macrophage subtype, or myofibroblast subtypes if there is an increase in the amount of mRNA in the sample relative to the control level. In some embodiments, the biopsy sample is a fine needle aspiration (FNA) biopsy sample. In some embodiments, a portion of the genetic material in the FNA biopsy sample is from fibroblasts. In some embodiments, the subject is being evaluated for presence, metastasis, and/or recurrence of thyroid cancer. In some embodiments, the biopsy sample is taken from a tumor microenvironment.

Embodiments of the presently-disclosed subject matter include a method of detecting expression of genes in a fine needle aspiration (FNA) biopsy sample from a subject, which comprises the steps of determining expression levels in the FNA biopsy sample of each of the gene of a fibroblast gene signature, wherein the fibroblast gene signature comprises 3-80 of the genes set forth in Table 1A, Table 1B, Table 1C, Table 1D, Table 1E, Table 2A, Table 2B, Table 2C, Table 2D, Table 2E, Table 2F, Table 3, Table 4, and Table 5; wherein the expression levels of each of the genes are determined by sequencing genetic material in the FNA biopsy sample. In some embodiments, a portion of the genetic material in the FNA biopsy sample is from fibroblasts. In some embodiments, the subject is being evaluated for presence, metastasis, and/or recurrence of thyroid cancer. In some embodiments, the biopsy sample is taken from a tumor microenvironment. In some embodiments, the method further comprises comparing the expression levels to expression levels for each gene in a control sample from a normal healthy individual or a benign individual, wherein overexpression of the genes in the FNA biopsy sample relative to the control is associated with high risk of presence, metastasis, and/or recurrence of thyroid cancer.

Embodiments of the presently-disclosed subject matter include a treatment method, which comprises determining expression levels in a fine needle aspiration (FNA) biopsy sample from a subject of each gene of a fibroblast gene signature, wherein the fibroblast gene signature comprises 3-80 of the genes set forth in Table 1A, Table 1B, Table 1C, Table 1D, Table 1E, Table 2A, Table 2B, Table 2C, Table 2D, Table 2E, Table 2F, Table 3, Table 4, and Table 5; wherein the expression levels of each of the genes are determined by sequencing genetic material in the FNA biopsy sample; comparing the expression levels to expression levels for each gene in a control sample from a normal healthy individual or a benign individual; and administering radioactive iodine to the subject when there is overexpression of the genes in the FNA biopsy sample relative expression levels of the genes in a control sample from a normal healthy individual or a benign individual.

In some embodiments of the methods as disclosed herein, the gene signature comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 genes selected from the genes set forth in Table 1A, Table 1B, Table 1C, Table 1D, Table 1E, Table 2A, Table 2B, Table 2C, Table 2D, Table 2E, Table 2F, Table 3, Table 4, and Table 5.

TABLE 1A
Genes for detection in fine needle aspiration (FNA) biopsy sample
myCAF_Genes

| | | | | |
|---|---|---|---|---|
| POSTN | MMP9 | CDH11 | TNFRSF6B | SLC16A3 |
| MMP11 | SFRP2 | DIO2 | MXRA5 | ANTXR1 |
| CST1 | CTSK | CD82 | PDPN | LRRC15 |
| COL1A1 | CXCL1 | FAP | TNFSF10 | EPSTI1 |
| CTHRC1 | COL6A3 | SPARC | MDK | SAT1 |
| COL11A1 | COL5A2 | COL12A1 | GREM1 | CXCL10 |
| CXCL8 | SULF1 | SDC1 | S100A16 | SPON2 |
| VCAN | MMP13 | COMP | TNFAIP6 | NNMT |
| COL3A1 | CCN2 | HTRA1 | PLAUR | RCN1 |
| MFAP2 | LUM | COL6A2 | PKM | CALU |
| HSPA6 | CCN1 | IFI27 | RCN3 | CYP1B1 |
| COL1A2 | COL5A1 | LGALS1 | CXCL6 | P4HB |
| INHBA | AEBP1 | ADM | SPHK1 | ADAM12 |
| THBS2 | CTSB | FTH1 | ISLR | SOD2 |
| COL10A1 | IL32 | PLOD2 | MMP7 | LY6E |
| COL6A1 | IGF2 | SERPINH1 | IFI6 | NBL1 |
| COL8A1 | RARRES2 | MMP2 | SERPINE1 | ENO1 |
| FN1 | PLAU | TMEM158 | H19 | ADAMTS2 |
| MMP14 | MMP1 | GJA1 | TMSB10 | CNN2 |
| | | | | RUNX2 |

TABLE 1B
Genes for detection in fine needle aspiration (FNA) biopsy sample
iCAF_Genes

| | | | | |
|---|---|---|---|---|
| APOD | IGF1 | PTGDS | GDF10 | SFRP1 |
| MYOC | PODN | ABCA6 | CTSF | ADD3 |
| CXCL14 | IGFBP6 | ABCA10 | SVEP1 | PMP22 |
| CFD | SELENOP | CYBRD1 | COL15A1 | PSAP |
| GSN | OGN | FGF7 | PLPP3 | DHRS3 |
| CXCL12 | ABI3BP | CST3 | LUM | PLXDC2 |
| PLA2G2A | ANGPTL1 | CCL4 | HLA-DRB1 | PLTP |
| C3 | NOV | PDGFRA | LSP1 | HSPB6 |
| FBLN1 | C1S | SERPINE2 | BOC | RNASE4 |
| CCDC80 | CLU | CTGF | ANXA1 | TSC22D3 |
| EFEMP1 | F3 | PDGFRL | AKR1C1 | LGALS3 |
| FGL2 | GPNMB | OSR1 | NFIA | SERPING1 |
| DPT | PRELP | EPHX1 | COL14A1 | ENG |
| DCN | PI16 | NFIB | METTL7A | LAMA2 |
| LTBP4 | SFRP2 | SUCNR1 | COLEC12 | PRNP |
| CFH | TNXB | S100A10 | ARL6IP5 | USP53 |
| ADH1B | C1R | SLIT2 | HSPG2 | TSPAN8 |
| ABCA8 | TFF3 | SCARA5 | F10 | ALDH2 |
| SRPX | ZFP36L2 | PTN | FAM180B | C16orf89 |
| MFAP4 | LRP1 | SSPN | CRISPLD1 | NFIX |
| FBLN2 | CD34 | FLRT2 | TMEM119 | IGSF10 |
| C7 | ALDH1A1 | IGFBP3 | CD74 | TIMP3 |
| MGP | MMP2 | PIK3R1 | ABCA9 | TG |
| GPC3 | OMD | GAS1 | MGST1 | IGF2.1 |
| SFRP4 | LEPR | CDO1 | WISP2 | DIO3OS |
| ITM2A | SERPINF1 | PID1 | ISLR | AHNAK |
| CHRDL1 | FMO2 | CPXM2 | ELN | TSHZ2 |
| FBLN5 | EMP1 | | SPRY1 | MATN2 |

TABLE 1C
Genes for detection in fine needle aspiration (FNA) biopsy sample
APOE_CAF_Genes

| | | | | |
|---|---|---|---|---|
| PRKG1 | PLCL1 | PARD3 | NEAT1 | ZBTB20 |
| APOE | CCDC102B | COL4A1 | EBF1 | RNF152 |
| SOX5 | ARIH1 | CACNA1C | UTRN | CACNB2 |
| DLC1 | NRXN3 | APOC1 | APBB2 | COL4A2 |
| PTPRG | RASAL2 | UACA | ZSWIM6 | |
| PEAK1 | NRG3 | RBMS3 | | |

TABLE 1D
Genes for detection in fine needle aspiration (FNA) biopsy sample
iPVCAF_Genes

| | | | | |
|---|---|---|---|---|
| RGS5 | HIGD1B | ARHGDIB | THY1 | FAM213A |
| FABP4 | COX412 | TPPP3 | FAM162B | MYO1B |
| CD36 | LHFP | C20orf27 | CYGB | KCNJ8 |
| STEAP4 | FABP5 | PMEPA1 | TINAGL1 | MARCKSL1 |
| NDUFA4L2 | GJA4 | MEF2C | CHN1 | ARHGAP15 |
| | | | | GMFG |

TABLE 1E
Genes for detection in fine needle aspiration (FNA) biopsy sample
dPVCAF_Genes

| | | | | |
|---|---|---|---|---|
| MYH11 | WFDC1 | CDKN1A | CYCS | NDUFA4 |
| ACTA2 | DSTN | CNN1 | TPM1 | CRYAB |
| RERGL | LBH | MYLK | NRARP | ACTN4 |
| MUSTN1 | NET1 | RCAN2 | MFGE8 | PTP4A3 |
| TAGLN | CRIP1 | C11orf96 | ADAMTS1 | MT1X |
| SORBS2 | PHLDA2 | CSRP2 | FLNA | CRIP2 |
| MYL9 | MCAM | GADD45B | FRZB | ID1 |
| BCAM | PPP1R14A | MT1M | TINAGL1 | GPRC5C |
| TPM2 | CSRP1 | LMOD1 | CAV1 | BTG2 |
| PLN | CASQ2 | FAM107B | MAP3K20 | ATF3 |
| ADIRF | ACTG2 | JAG1 | | |

TABLE 2A
Genes for detection in fine needle aspiration (FNA) biopsy sample

| LUM_CAF_Genes | avg_log2FC | pct. 1 | pct. 2 | p_val_adj |
|---|---|---|---|---|
| SFRP2 | 3.68486709 | 0.893 | 0.135 | 0 |
| COL10A1 | 2.6883945 | 0.478 | 0.046 | 0 |
| LUM | 3.43790728 | 0.988 | 0.509 | 9.98E−232 |
| LAMP5 | 1.6697064 | 0.289 | 0.028 | 1.84E−186 |
| CTHRC1 | 2.7555268 | 0.925 | 0.551 | 3.07E−185 |
| COL11A1 | 2.94571183 | 0.485 | 0.096 | 1.40E−167 |
| COL1A1 | 2.47988478 | 1 | 0.742 | 2.21E−165 |
| MMP13 | 1.6683909 | 0.174 | 0.012 | 3.79E−149 |
| COL8A1 | 2.43450804 | 0.535 | 0.132 | 5.61E−149 |
| DCN | 1.83202624 | 0.968 | 0.577 | 1.60E−146 |
| COL3A1 | 1.93491823 | 0.993 | 0.783 | 9.96E−142 |
| CTSK | 2.22356907 | 0.609 | 0.189 | 5.72E−130 |
| ITGBL1 | 2.04311422 | 0.403 | 0.082 | 4.93E−125 |
| COMP | 2.62248975 | 0.299 | 0.045 | 1.79E−124 |
| SFRP4 | 1.86448825 | 0.664 | 0.263 | 1.07E−103 |
| UQCR11.1 | 1.36675925 | 0.393 | 0.08 | 1.17E−103 |
| NBL1 | 2.53981121 | 0.664 | 0.305 | 3.63E−103 |
| SPARC | 1.43961864 | 0.99 | 0.889 | 3.89E−103 |
| VCAN | 1.98095449 | 0.841 | 0.545 | 2.73E−101 |
| POSTN | 2.79786752 | 0.779 | 0.462 | 5.69E−94 |
| MMP11 | 2.5924791 | 0.356 | 0.081 | 1.40E−90 |
| AEBP1 | 2.11140819 | 0.749 | 0.537 | 9.25E−81 |
| TMEM66 | 1.06351432 | 0.264 | 0.048 | 2.01E−79 |
| THBS2 | 2.16920594 | 0.664 | 0.378 | 3.78E−77 |
| COL6A3 | 1.3712738 | 0.863 | 0.639 | 6.90E−69 |
| HTRA1 | 2.26693479 | 0.692 | 0.483 | 1.57E−67 |
| ASPN | 2.11656788 | 0.724 | 0.418 | 1.95E−61 |
| C1S | 1.40526535 | 0.652 | 0.332 | 8.97E−61 |
| COL5A2 | 1.55201118 | 0.754 | 0.581 | 1.74E−54 |
| TAGLN | 1.20040494 | 0.803 | 0.548 | 4.21E−54 |
| LRRC15 | 1.03242688 | 0.159 | 0.029 | 1.98E−48 |
| FAM198B | 1.44909282 | 0.311 | 0.101 | 7.11E−47 |
| PTRF | 1.1398741 | 0.463 | 0.192 | 1.07E−42 |
| MMP2 | 1.68698396 | 0.644 | 0.499 | 2.42E−40 |
| FBLN1 | 1.03544809 | 0.726 | 0.523 | 5.22E−39 |
| OMD | 1.2893487 | 0.284 | 0.095 | 8.45E−37 |
| DIO2 | 1.33654026 | 0.229 | 0.065 | 1.18E−36 |
| CYP1B1 | 1.51552259 | 0.316 | 0.118 | 1.19E−35 |
| TIMP3 | 1.93840484 | 0.632 | 0.46 | 4.15E−35 |
| FGF7 | 1.38158592 | 0.328 | 0.128 | 4.84E−33 |
| SDC1 | 1.70775541 | 0.336 | 0.149 | 7.85E−33 |
| MXRA5 | 1.6954208 | 0.483 | 0.303 | 2.35E−31 |
| PPIC | 1.41952812 | 0.622 | 0.504 | 3.64E−31 |
| LINC00657 | 1.00311432 | 0.328 | 0.126 | 3.85E−31 |
| CD99 | 1.09206095 | 0.734 | 0.716 | 2.93E−29 |
| IGF2 | 1.04129386 | 0.142 | 0.033 | 2.76E−28 |
| CXCL14 | 1.35585731 | 0.241 | 0.08 | 6.39E−28 |
| KIAA1217 | 1.161229 | 0.254 | 0.099 | 2.40E−25 |
| CTGF | 1.52136068 | 0.639 | 0.53 | 2.67E−24 |
| COL5A1 | 1.23142674 | 0.622 | 0.507 | 9.52E−23 |
| ISLR | 1.58143429 | 0.44 | 0.29 | 5.50E−22 |
| SULF1 | 1.69498074 | 0.49 | 0.35 | 7.16E−22 |
| IGFBP3 | 1.73210925 | 0.328 | 0.17 | 1.58E−21 |
| CDH11 | 1.21479541 | 0.53 | 0.381 | 3.27E−20 |
| SERPINF1 | 1.06255387 | 0.637 | 0.537 | 3.29E−20 |
| PALLD | 1.43737584 | 0.502 | 0.392 | 4.18E−19 |
| IGFBP4 | 1.05348464 | 0.657 | 0.578 | 4.85E−19 |
| TIMP2 | 1.21999287 | 0.612 | 0.536 | 3.52E−18 |
| GJA1 | 1.44013137 | 0.368 | 0.233 | 4.10E−16 |
| VCAM1 | 1.01824972 | 0.204 | 0.088 | 1.75E−13 |
| PRSS23 | 1.10796218 | 0.55 | 0.43 | 6.32E−13 |
| ANTXR1 | 1.49695388 | 0.46 | 0.395 | 1.66E−11 |
| CCDC80 | 1.12305346 | 0.592 | 0.564 | 2.59E−10 |
| MMP14 | 1.19215093 | 0.517 | 0.438 | 6.99E−10 |
| MXRA8 | 1.06919339 | 0.555 | 0.533 | 1.17E−09 |
| INHBA | 1.04447655 | 0.455 | 0.35 | 4.05E−09 |
| COL12A1 | 1.80281586 | 0.47 | 0.424 | 4.57E−08 |
| FAP | 1.26618024 | 0.43 | 0.381 | 1.14E−07 |
| VMP1 | 1.02003926 | 0.567 | 0.592 | 3.59E−07 |
| RAB31 | 1.30299685 | 0.448 | 0.411 | 5.73E−07 |
| OLFML3 | 1.11487886 | 0.368 | 0.292 | 3.70E−06 |
| FNDC1 | 1.04502589 | 0.206 | 0.122 | 1.45E−05 |
| PRELP | 1.06054915 | 0.192 | 0.106 | 4.41E−05 |
| EDIL3 | 1.13831661 | 0.301 | 0.232 | 6.80E−04 |
| ITGB5 | 1.16376302 | 0.326 | 0.268 | 7.46E−04 |
| ANKH | 1.17516261 | 0.316 | 0.258 | 0.00114162 |
| THBS1 | 1.18307175 | 0.261 | 0.185 | 0.0018194 |

TABLE 2B
Genes for detection in fine needle aspiration (FNA) biopsy sample

| RARRES2_CAF_Genes | avg_log2FC | pct. 1 | pct. 2 | p_val_adj |
|---|---|---|---|---|
| FMO1 | 1.03063573 | 0.284 | 0.021 | 0 |
| CHI3L1 | 1.37123591 | 0.247 | 0.024 | 0 |
| IL24 | 2.05913677 | 0.282 | 0.004 | 0 |
| IL1R1 | 1.04225264 | 0.578 | 0.157 | 0 |
| COL6A3 | 1.74092477 | 0.931 | 0.627 | 0 |
| TMEM158 | 1.61659769 | 0.69 | 0.216 | 0 |
| COL7A1 | 1.41162894 | 0.473 | 0.064 | 0 |
| MME | 1.75103703 | 0.676 | 0.158 | 0 |
| APOD | 1.73844648 | 0.463 | 0.092 | 0 |
| CXCL8 | 3.44943473 | 0.515 | 0.12 | 0 |
| CXCL6 | 2.29571991 | 0.461 | 0.094 | 0 |
| CXCL1 | 2.15538187 | 0.487 | 0.106 | 0 |
| IL7R | 1.10801742 | 0.363 | 0.051 | 0 |
| IL6 | 2.12971318 | 0.357 | 0.053 | 0 |
| AEBP1 | 1.66641769 | 0.88 | 0.521 | 0 |
| PTGDS | 1.23611855 | 0.304 | 0.028 | 0 |
| CD82 | 1.28422893 | 0.627 | 0.18 | 0 |
| MMP3 | 2.50567893 | 0.204 | 0.01 | 0 |
| C1S | 1.44193504 | 0.875 | 0.308 | 0 |
| C1R | 1.44034081 | 0.858 | 0.392 | 0 |
| EPSTI1 | 1.18611268 | 0.517 | 0.123 | 0 |
| GREM1 | 1.47522763 | 0.433 | 0.085 | 0 |
| FGF7 | 1.60149153 | 0.611 | 0.104 | 0 |
| CA12 | 1.84348833 | 0.662 | 0.145 | 0 |
| MMP9 | 2.22124402 | 0.412 | 0.04 | 0 |
| COL6A1 | 2.06637216 | 0.917 | 0.707 | 0 |
| COL6A2 | 2.34118403 | 0.945 | 0.804 | 0 |
| DCN | 1.34784122 | 0.929 | 0.565 | 1.99E−292 |
| ADM | 1.9067435 | 0.675 | 0.23 | 7.17E−292 |
| LUM | 1.62348752 | 0.88 | 0.498 | 3.02E−286 |
| GJA1 | 1.29212351 | 0.63 | 0.213 | 3.21E−286 |
| CTSK | 1.24404579 | 0.607 | 0.174 | 1.99E−282 |
| CEBPB | 1.52949725 | 0.82 | 0.429 | 2.97E−276 |
| TDO2 | 1.12100053 | 0.411 | 0.084 | 2.23E−275 |
| SOD2 | 1.86373276 | 0.717 | 0.316 | 4.43E−273 |
| NEAT1 | 1.56708211 | 0.949 | 0.716 | 1.99E−262 |
| MMP14 | 1.64856386 | 0.804 | 0.419 | 4.33E−240 |
| SLC16A3 | 1.37521522 | 0.698 | 0.299 | 8.02E−240 |
| NAMPT | 1.5104263 | 0.703 | 0.313 | 1.24E−237 |
| IL32 | 1.30065879 | 0.672 | 0.267 | 3.89E−233 |
| HLA-B | 1.58922607 | 0.914 | 0.723 | 1.81E−231 |
| MMP2 | 1.46836636 | 0.816 | 0.484 | 1.05E−230 |
| LOXL2 | 1.26158596 | 0.656 | 0.275 | 4.34E−230 |
| IER3 | 2.36812369 | 0.711 | 0.391 | 1.32E−229 |
| NNMT | 1.54458724 | 0.807 | 0.441 | 1.12E−228 |
| THBS2 | 1.30360025 | 0.755 | 0.362 | 2.00E−222 |
| CLEC2B | 1.27179896 | 0.618 | 0.229 | 2.36E−222 |
| RARRES2 | 2.20042272 | 0.711 | 0.4 | 9.24E−222 |
| FTH1 | 1.92457095 | 0.991 | 0.979 | 1.73E−220 |
| CCL2 | 1.66607472 | 0.461 | 0.129 | 2.08E−220 |
| EMILIN1 | 1.08421875 | 0.621 | 0.253 | 1.26E−218 |
| WNT5A | 1.48150699 | 0.581 | 0.23 | 2.75E−218 |
| TNFSF10 | 1.11392358 | 0.569 | 0.195 | 8.88E−217 |
| NBL1 | 1.07086227 | 0.682 | 0.291 | 1.46E−214 |
| HLA-A | 1.43477612 | 0.905 | 0.739 | 1.44E−206 |
| RND3 | 1.48229391 | 0.753 | 0.396 | 4.80E−203 |
| GAS1 | 1.17850688 | 0.583 | 0.23 | 1.74E−199 |
| DUSP1 | 1.37726384 | 0.855 | 0.611 | 3.16E−197 |
| SAT1 | 1.62998621 | 0.878 | 0.659 | 1.35E−194 |
| CXCL3 | 1.53878823 | 0.434 | 0.123 | 2.60E−188 |
| STC1 | 1.5442482 | 0.394 | 0.105 | 2.92E−188 |
| TWIST2 | 1.25902096 | 0.528 | 0.217 | 2.87E−182 |
| NDRG1 | 1.20304185 | 0.661 | 0.307 | 1.03E−179 |
| TIMP1 | 2.2775618 | 0.95 | 0.882 | 1.01E−178 |
| THBS1 | 1.09206982 | 0.495 | 0.168 | 3.79E−178 |
| NFKBIZ | 1.00025431 | 0.614 | 0.267 | 1.48E−169 |
| COL5A2 | 1.20781426 | 0.819 | 0.571 | 2.48E−165 |
| RARRES1 | 1.06072385 | 0.321 | 0.081 | 1.11E−163 |
| TYMP | 1.2936995 | 0.727 | 0.456 | 3.67E−162 |
| SELENOM | 1.51013245 | 0.665 | 0.421 | 2.02E−159 |
| CXCL5 | 1.22183395 | 0.328 | 0.082 | 6.55E−159 |
| ANKRD28 | 1.05272834 | 0.639 | 0.329 | 9.94E−159 |
| HLA-C | 1.14531198 | 0.89 | 0.697 | 1.34E−156 |
| INHBA | 1.27511981 | 0.666 | 0.334 | 1.60E−156 |
| LY6E | 1.1378019 | 0.805 | 0.531 | 1.74E−155 |
| PLOD2 | 1.05665947 | 0.678 | 0.39 | 1.55E−140 |
| COL1A1 | 1.16224523 | 0.937 | 0.736 | 1.97E−140 |
| CYTOR | 1.05978307 | 0.643 | 0.347 | 7.11E−138 |
| SERPINE1 | 1.05242118 | 0.586 | 0.259 | 1.74E−126 |
| DIO2 | 1.45745293 | 0.245 | 0.058 | 5.96E−126 |
| SLC2A3 | 1.10525906 | 0.667 | 0.391 | 7.55E−122 |
| PLIN2 | 1.28674133 | 0.612 | 0.337 | 1.28E−119 |
| ANGPTL4 | 1.19572556 | 0.631 | 0.328 | 2.89E−118 |
| HAS2 | 1.22776741 | 0.481 | 0.226 | 1.49E−117 |
| MT2A | 1.49146215 | 0.941 | 0.838 | 2.53E−116 |
| TNFAIP6 | 1.36453695 | 0.655 | 0.403 | 9.68E−110 |
| PDLIM4 | 1.00915836 | 0.553 | 0.297 | 1.14E−107 |
| TIMP3 | 1.51055877 | 0.727 | 0.449 | 1.85E−106 |
| COL3A1 | 1.04840424 | 0.937 | 0.778 | 1.21E−102 |
| LGALS1 | 1.03217603 | 0.961 | 0.958 | 3.32E−98 |
| CXCL12 | 1.08062116 | 0.436 | 0.196 | 6.10E−89 |
| DDIT4 | 1.09361437 | 0.631 | 0.399 | 5.52E−85 |
| MMP1 | 1.65775909 | 0.33 | 0.127 | 2.61E−82 |
| IGFBP2 | 1.11285724 | 0.398 | 0.26 | 1.10E−28 |

TABLE 2C
Genes for detection in fine needle aspiration (FNA) biopsy sample

| CXCL14_CAF_Genes | avg_log2FC | pct. 1 | pct. 2 | p_val_adj |
|---|---|---|---|---|
| PODN | 1.70377288 | 0.572 | 0.053 | 0 |
| LEPR | 1.92208603 | 0.474 | 0.049 | 0 |
| CTSK | 1.65164433 | 0.653 | 0.172 | 0 |
| S100A10 | 1.79036148 | 0.96 | 0.616 | 0 |
| DPT | 2.26176446 | 0.665 | 0.182 | 0 |
| ANGPTL1 | 1.63242753 | 0.444 | 0.046 | 0 |
| PRELP | 2.1037352 | 0.635 | 0.079 | 0 |
| CD34 | 1.70285616 | 0.548 | 0.049 | 0 |
| EFEMP1 | 2.48447374 | 0.807 | 0.286 | 0 |
| ACKR3 | 2.33430262 | 0.621 | 0.078 | 0 |
| FBLN2 | 2.78960064 | 0.843 | 0.111 | 0 |
| ABI3BP | 1.99361213 | 0.647 | 0.155 | 0 |
| CCDC80 | 3.5859126 | 0.983 | 0.542 | 0 |
| PCOLCE2 | 1.87558778 | 0.53 | 0.09 | 0 |
| APOD | 3.40501265 | 0.608 | 0.085 | 0 |
| SFRP2 | 3.86564767 | 0.829 | 0.113 | 0 |
| CXCL14 | 4.4470362 | 0.713 | 0.049 | 0 |
| TNXB | 2.62630799 | 0.755 | 0.093 | 0 |
| SFRP4 | 4.44108205 | 0.859 | 0.239 | 0 |
| FGL2 | 1.4787434 | 0.366 | 0.02 | 0 |
| ITM2A | 2.9034187 | 0.784 | 0.077 | 0 |
| CHRDL1 | 1.80675629 | 0.537 | 0.065 | 0 |
| PDGFRL | 1.99847541 | 0.647 | 0.182 | 0 |
| SCARA5 | 1.79372008 | 0.523 | 0.039 | 0 |
| OGN | 1.72657078 | 0.773 | 0.274 | 0 |
| OMD | 1.94338935 | 0.578 | 0.073 | 0 |
| GSN | 3.2673668 | 0.976 | 0.708 | 0 |
| LRRN4CL | 1.17120887 | 0.385 | 0.043 | 0 |
| CPXM2 | 1.11315169 | 0.354 | 0.033 | 0 |
| C1S | 2.49632178 | 0.924 | 0.307 | 0 |
| C1R | 2.28273421 | 0.903 | 0.391 | 0 |
| MFAP5 | 2.55254353 | 0.876 | 0.331 | 0 |
| IGFBP6 | 2.56662966 | 0.923 | 0.547 | 0 |
| LUM | 2.50232776 | 0.949 | 0.496 | 0 |
| DCN | 3.58289432 | 0.999 | 0.562 | 0 |
| F10 | 1.33603089 | 0.422 | 0.052 | 0 |
| FBLN5 | 1.87074697 | 0.666 | 0.157 | 0 |
| FGF7 | 1.58558276 | 0.583 | 0.107 | 0 |
| SERPINF1 | 2.15409823 | 0.893 | 0.52 | 0 |
| MFAP4 | 2.69705549 | 0.862 | 0.262 | 0 |
| ABCA10 | 1.0501255 | 0.257 | 0.02 | 0 |
| CST3 | 1.63146144 | 0.992 | 0.879 | 0 |
| WISP2 | 2.50773546 | 0.619 | 0.074 | 0 |
| SLPI | 2.03421533 | 0.598 | 0.148 | 0 |
| CFD | 3.96924047 | 0.916 | 0.218 | 0 |
| C3 | 3.24115556 | 0.748 | 0.088 | 0 |
| FBLN1 | 3.01330455 | 0.958 | 0.504 | 0 |
| PLA2G2A | 4.81525237 | 0.49 | 0.028 | 0 |
| PRG4 | 1.80612296 | 0.322 | 0.041 | 0 |
| CLEC3B | 1.5935288 | 0.465 | 0.058 | 0 |
| PI16 | 2.78589687 | 0.681 | 0.041 | 0 |
| IGF1 | 2.15350864 | 0.592 | 0.168 | 0 |
| CILP | 1.37729005 | 0.325 | 0.017 | 0 |
| ADH1B | 1.79072944 | 0.312 | 0.022 | 0 |
| FAM180B | 1.05693925 | 0.294 | 0.024 | 0 |
| IGF2 | 1.19707387 | 0.277 | 0.022 | 0 |
| ABCA8 | 1.92397597 | 0.605 | 0.196 | 5.02E−292 |
| HAS1 | 1.45676592 | 0.207 | 0.018 | 4.63E−286 |
| SERPING1 | 1.56929249 | 0.877 | 0.495 | 9.73E−278 |
| MGST1 | 1.71940434 | 0.691 | 0.253 | 1.17E−276 |
| CFH | 2.19224821 | 0.771 | 0.393 | 3.56E−276 |
| S100A4 | 1.35721742 | 0.977 | 0.597 | 6.63E−274 |
| ITM2B | 1.33190991 | 0.978 | 0.863 | 2.80E−272 |
| GPNMB | 1.81153562 | 0.752 | 0.366 | 8.23E−265 |
| FSTL1 | 1.63882332 | 0.898 | 0.608 | 9.50E−265 |
| FBN1 | 1.83963611 | 0.85 | 0.511 | 1.93E−262 |
| MMP2 | 1.78512472 | 0.808 | 0.486 | 5.19E−254 |
| KLF4 | 1.74578932 | 0.593 | 0.21 | 4.97E−246 |
| SEPP1 | 1.2922523 | 0.673 | 0.212 | 2.61E−242 |
| MGP | 1.55452926 | 0.986 | 0.663 | 2.73E−237 |
| TIMP3 | 1.6337655 | 0.829 | 0.444 | 3.52E−234 |
| CYBRD1 | 1.64054722 | 0.746 | 0.431 | 2.98E−229 |
| PMP22 | 1.53229565 | 0.803 | 0.519 | 3.09E−225 |
| MEDAG | 1.17962404 | 0.421 | 0.111 | 1.00E−217 |
| CD99 | 1.25341113 | 0.884 | 0.707 | 1.81E−211 |
| PTGIS | 1.2510986 | 0.477 | 0.147 | 1.93E−205 |
| GAS1 | 1.50455017 | 0.58 | 0.231 | 7.49E−204 |
| PPIC | 1.55258069 | 0.757 | 0.492 | 4.21E−198 |
| FOS | 1.42880721 | 0.905 | 0.704 | 1.20E−197 |
| ANXA2 | 1.10012638 | 0.934 | 0.713 | 1.23E−196 |
| TIMP2 | 1.35024922 | 0.809 | 0.523 | 8.57E−196 |
| DUSP1 | 1.37665651 | 0.869 | 0.611 | 2.41E−187 |
| CELF2 | 1.05346392 | 0.447 | 0.138 | 1.03E−186 |
| GPX3 | 1.64166932 | 0.519 | 0.176 | 1.11E−185 |
| NOV | 1.72515371 | 0.256 | 0.048 | 1.53E−175 |
| NOVA1 | 1.09841254 | 0.35 | 0.088 | 5.64E−173 |
| SEMA3C | 1.58833137 | 0.545 | 0.24 | 1.91E−167 |
| NNMT | 1.29289186 | 0.792 | 0.443 | 5.65E−165 |
| ZFP36 | 1.52023069 | 0.795 | 0.562 | 4.04E−162 |
| ARL6IP5 | 1.30123148 | 0.773 | 0.565 | 7.49E−161 |
| ABLIM1 | 1.13556122 | 0.413 | 0.135 | 9.48E−161 |
| ISLR | 1.39518476 | 0.567 | 0.278 | 9.86E−153 |
| DHRS3 | 1.3513066 | 0.476 | 0.187 | 7.71E−152 |
| CYP1B1 | 1.1495915 | 0.37 | 0.109 | 9.53E−152 |
| RAMP2 | 1.52330295 | 0.47 | 0.186 | 3.41E−151 |
| NR4A1 | 1.36600542 | 0.527 | 0.215 | 8.49E−151 |
| HSPG2 | 1.24289297 | 0.543 | 0.249 | 1.07E−148 |
| COL14A1 | 1.18319559 | 0.679 | 0.332 | 4.78E−147 |
| CEBPD | 1.50966843 | 0.71 | 0.462 | 7.26E−146 |
| NBL1 | 1.23854092 | 0.591 | 0.297 | 1.03E−144 |
| CXCL12 | 1.6248735 | 0.477 | 0.194 | 7.66E−141 |
| F3 | 1.51988027 | 0.426 | 0.16 | 4.05E−138 |
| HTRA3 | 1.52077089 | 0.548 | 0.272 | 1.72E−137 |
| ELN | 1.2320705 | 0.583 | 0.263 | 2.36E−137 |
| IGFBP5 | 1.36237226 | 0.808 | 0.47 | 7.50E−136 |
| JUNB | 1.20915102 | 0.848 | 0.69 | 1.66E−132 |
| ALDH2 | 1.05220988 | 0.408 | 0.154 | 9.61E−130 |
| ADD3 | 1.19976832 | 0.629 | 0.392 | 1.46E−125 |
| CD248 | 1.03684383 | 0.53 | 0.239 | 1.06E−124 |
| CD55 | 1.30608405 | 0.596 | 0.327 | 4.73E−121 |
| UAP1 | 1.36336137 | 0.523 | 0.274 | 9.21E−120 |
| RARRES1 | 1.2933029 | 0.286 | 0.084 | 3.41E−117 |
| RGCC | 1.20032089 | 0.304 | 0.091 | 1.37E−114 |
| HLA-DRB1 | 1.09928811 | 0.371 | 0.132 | 2.57E−114 |
| PROCR | 1.08686831 | 0.497 | 0.231 | 2.81E−113 |
| IGFBP4 | 1.23183395 | 0.759 | 0.57 | 5.22E−110 |
| MYADM | 1.1113322 | 0.68 | 0.482 | 2.50E−109 |
| EMP1 | 1.18827243 | 0.736 | 0.495 | 1.28E−105 |
| EGR1 | 1.18215109 | 0.724 | 0.53 | 9.55E−102 |
| OLFML3 | 1.00924631 | 0.501 | 0.283 | 2.66E−91 |
| PIK3R1 | 1.07748473 | 0.457 | 0.231 | 2.08E−90 |
| AHNAK | 1.0539179 | 0.745 | 0.581 | 2.48E−90 |
| SRPX | 1.25184368 | 0.484 | 0.267 | 3.63E−89 |
| ZFP36L2 | 1.02209086 | 0.715 | 0.508 | 1.62E−85 |
| CYB5A | 1.03698881 | 0.527 | 0.329 | 1.93E−85 |
| TGFBR2 | 1.03781648 | 0.499 | 0.286 | 8.43E−84 |
| PLPP3 | 1.06050929 | 0.418 | 0.209 | 1.01E−82 |
| TSC22D3 | 1.10761017 | 0.698 | 0.528 | 8.32E−79 |
| THBS1 | 1.3589131 | 0.382 | 0.175 | 2.00E−78 |
| BTG2 | 1.13615746 | 0.413 | 0.201 | 1.41E−75 |
| FHL1 | 1.12469711 | 0.584 | 0.431 | 1.00E−74 |
| PTGDS | 1.23637372 | 0.156 | 0.037 | 1.24E−73 |
| MT1X | 1.17832894 | 0.689 | 0.52 | 3.57E−73 |
| THBS4 | 1.33714534 | 0.163 | 0.041 | 5.10E−73 |
| CTGF | 1.15729872 | 0.74 | 0.521 | 6.20E−72 |
| AKAP12 | 1.18466093 | 0.588 | 0.434 | 9.83E−60 |
| GADD45B | 1.21741444 | 0.624 | 0.513 | 9.83E−50 |
| MTRNR2L12 | 1.3247366 | 0.525 | 0.418 | 2.53E−40 |
| COMP | 1.72339087 | 0.138 | 0.045 | 2.60E−40 |
| CYR61 | 1.0281768 | 0.598 | 0.495 | 8.88E−40 |
| PLCG2 | 1.03653824 | 0.298 | 0.223 | 3.85E−12 |

TABLE 2D
Genes for detection in fine needle aspiration (FNA) biopsy sample

| ACKR3_CAF_Genes | avg_log2FC | pct. 1 | pct. 2 | p_val_adj |
|---|---|---|---|---|
| FABP3 | 2.18803068 | 0.619 | 0.124 | 0 |
| LEPR | 1.27981689 | 0.551 | 0.053 | 0 |
| TGFBR3 | 1.89241812 | 0.839 | 0.166 | 0 |
| S100A10 | 2.92800195 | 1 | 0.62 | 0 |
| S100A11 | 1.45417559 | 0.997 | 0.869 | 0 |
| S100A6 | 2.00501648 | 0.999 | 0.883 | 0 |
| S100A4 | 2.71729089 | 1 | 0.602 | 0 |
| CADM3 | 1.57618994 | 0.58 | 0.013 | 0 |
| LINC01133 | 2.16911463 | 0.859 | 0.093 | 0 |
| UAP1 | 1.77967681 | 0.847 | 0.265 | 0 |
| DPT | 1.81791727 | 0.795 | 0.184 | 0 |
| PRELP | 1.43329922 | 0.777 | 0.082 | 0 |
| CD55 | 3.06938929 | 0.98 | 0.317 | 0 |
| CD34 | 2.05271314 | 0.873 | 0.044 | 0 |
| VIT | 1.48376397 | 0.668 | 0.022 | 0 |
| EFEMP1 | 2.40844912 | 0.97 | 0.288 | 0 |
| GYPC | 1.69601778 | 0.886 | 0.373 | 0 |
| AOX1 | 1.01667788 | 0.486 | 0.052 | 0 |
| IGFBP5 | 2.87964843 | 0.995 | 0.468 | 0 |
| EFHD1 | 1.10238366 | 0.674 | 0.095 | 0 |
| ACKR3 | 3.41820152 | 0.947 | 0.074 | 0 |
| CHL1 | 1.09416968 | 0.484 | 0.018 | 0 |
| FBLN2 | 2.59137285 | 0.982 | 0.116 | 0 |
| ABI3BP | 1.25908745 | 0.727 | 0.159 | 0 |
| CCDC80 | 2.00497117 | 0.997 | 0.548 | 0 |
| FSTL1 | 2.57508561 | 0.993 | 0.609 | 0 |
| ACKR4 | 1.05265419 | 0.468 | 0.025 | 0 |
| PCOLCE2 | 3.75280735 | 0.986 | 0.079 | 0 |
| HTRA3 | 2.17658701 | 0.826 | 0.265 | 0 |
| SH3D19 | 1.21432173 | 0.691 | 0.148 | 0 |
| C1QTNF3 | 2.09924477 | 0.935 | 0.249 | 0 |
| PPIC | 1.8880812 | 0.97 | 0.488 | 0 |
| GFPT2 | 1.29672155 | 0.676 | 0.138 | 0 |
| TNXB | 3.21949206 | 0.981 | 0.095 | 0 |
| CREB5 | 1.65081535 | 0.859 | 0.288 | 0 |
| SEMA3C | 2.60111289 | 0.915 | 0.23 | 0 |
| CD99 | 1.66309888 | 0.988 | 0.706 | 0 |
| ITM2A | 2.23119641 | 0.918 | 0.083 | 0 |
| CHRDL1 | 1.32259389 | 0.653 | 0.068 | 0 |
| PDGFRL | 2.08463622 | 0.874 | 0.181 | 0 |
| SCARA5 | 2.09607616 | 0.888 | 0.033 | 0 |
| OSR2 | 1.24391859 | 0.551 | 0.066 | 0 |
| NOV | 1.14837739 | 0.457 | 0.044 | 0 |
| GAS1 | 1.60365644 | 0.784 | 0.229 | 0 |
| OMD | 1.1218311 | 0.615 | 0.079 | 0 |
| KLF4 | 2.25515938 | 0.897 | 0.205 | 0 |
| GSN | 2.24624197 | 0.997 | 0.711 | 0 |
| LRRN4CL | 1.51553074 | 0.723 | 0.035 | 0 |
| CD248 | 1.59845413 | 0.855 | 0.231 | 0 |
| PDGFD | 1.54603829 | 0.709 | 0.055 | 0 |
| CELF2 | 1.17833516 | 0.693 | 0.134 | 0 |
| PLAC9 | 1.93528899 | 0.993 | 0.672 | 0 |
| ABLIM1 | 1.49731314 | 0.747 | 0.127 | 0 |
| CPXM2 | 1.06593788 | 0.514 | 0.032 | 0 |
| C1S | 1.60131614 | 0.97 | 0.314 | 0 |
| MFAP5 | 4.03288548 | 0.999 | 0.334 | 0 |
| MGST1 | 1.91959247 | 0.955 | 0.249 | 0 |
| IGFBP6 | 3.70153749 | 1 | 0.55 | 0 |
| DCN | 2.64979829 | 1 | 0.569 | 0 |
| MEDAG | 1.3735043 | 0.696 | 0.105 | 0 |
| CAB39L | 1.05961222 | 0.518 | 0.049 | 0 |
| F10 | 2.15782587 | 0.849 | 0.041 | 0 |
| TRAC | 1.28235229 | 0.546 | 0.082 | 0 |
| STXBP6 | 1.33570829 | 0.659 | 0.1 | 0 |
| NOVA1 | 1.8353331 | 0.731 | 0.078 | 0 |
| JDP2 | 1.57534499 | 0.743 | 0.171 | 0 |
| FBLN5 | 1.62574173 | 0.796 | 0.16 | 0 |
| FBN1 | 3.23654019 | 0.999 | 0.511 | 0 |
| ANXA2 | 1.78977222 | 0.997 | 0.714 | 0 |
| CTSH | 1.52989395 | 0.709 | 0.135 | 0 |
| ALDH1A3 | 1.26538976 | 0.539 | 0.064 | 0 |
| PCSK6 | 1.19016213 | 0.526 | 0.024 | 0 |
| HSD3B7 | 1.33020434 | 0.653 | 0.128 | 0 |
| GAS7 | 1.23148732 | 0.592 | 0.074 | 0 |
| MFAP4 | 3.50798003 | 0.992 | 0.266 | 0 |
| RAMP2 | 2.11182469 | 0.853 | 0.175 | 0 |
| ARL4D | 1.13809192 | 0.595 | 0.115 | 0 |
| C17orf58 | 2.3139746 | 0.834 | 0.173 | 0 |
| ABCA8 | 1.23176643 | 0.749 | 0.196 | 0 |
| ABCA9 | 1.07803727 | 0.536 | 0.058 | 0 |
| TIMP2 | 1.8544456 | 0.982 | 0.521 | 0 |
| METRNL | 1.72449568 | 0.849 | 0.284 | 0 |
| CST3 | 1.94470079 | 1 | 0.881 | 0 |
| PROCR | 1.62433352 | 0.862 | 0.221 | 0 |
| WISP2 | 1.74232943 | 0.714 | 0.079 | 0 |
| SLPI | 3.36073036 | 0.915 | 0.143 | 0 |
| PTGIS | 1.32051531 | 0.711 | 0.143 | 0 |
| CFD | 4.4428072 | 1 | 0.226 | 0 |
| TIMP3 | 1.58194864 | 0.978 | 0.444 | 0 |
| ADAMTS5 | 1.56589114 | 0.746 | 0.204 | 0 |
| CBR3 | 1.70130201 | 0.751 | 0.18 | 0 |
| PRG4 | 4.25510384 | 0.934 | 0.022 | 0 |
| CLEC3B | 3.16714684 | 0.943 | 0.046 | 0 |
| PI16 | 3.39099279 | 0.941 | 0.041 | 0 |
| RPS4Y1 | 1.1241297 | 0.839 | 0.178 | 0 |
| ADH1B | 1.37511359 | 0.327 | 0.026 | 0 |
| CYP4B1 | 1.38681946 | 0.519 | 0.009 | 0 |
| RP11-14N7.2 | 1.59413734 | 0.807 | 0.077 | 0 |
| MYEOV2 | 1.15500006 | 0.849 | 0.176 | 0 |
| CCDC109B | 1.01992666 | 0.549 | 0.03 | 0 |
| AC090498.1 | 1.07065229 | 0.712 | 0.089 | 0 |
| PRKCDBP | 1.2101313 | 0.889 | 0.2 | 0 |
| C10orf54 | 1.35531764 | 0.688 | 0.052 | 0 |
| VIMP | 1.10169683 | 0.803 | 0.135 | 0 |
| PTRF | 1.22183241 | 0.876 | 0.171 | 0 |
| FBLN1 | 1.67729354 | 0.982 | 0.51 | 4.72E−295 |
| DBN1 | 1.38881496 | 0.827 | 0.334 | 1.11E−293 |
| PLPP3 | 1.30046361 | 0.699 | 0.202 | 2.32E−293 |
| SMIM14 | 1.44358143 | 0.896 | 0.419 | 3.57E−293 |
| NT5E | 1.1417561 | 0.714 | 0.21 | 1.68E−288 |
| ADI1 | 1.53645285 | 0.891 | 0.435 | 1.81E−287 |
| SEPW1 | 1.13244213 | 0.896 | 0.244 | 1.71E−283 |
| LSP1 | 1.18176057 | 0.792 | 0.252 | 6.97E−281 |
| DCLK1 | 1.06856059 | 0.612 | 0.155 | 1.06E−280 |
| GPX4 | 1.48452619 | 0.986 | 0.722 | 2.22E−280 |
| C1R | 1.47525326 | 0.951 | 0.397 | 5.15E−279 |
| CD81 | 1.46002633 | 0.954 | 0.656 | 9.08E−279 |
| S100A13 | 1.58232674 | 0.955 | 0.599 | 1.73E−274 |
| GPNMB | 1.36418171 | 0.899 | 0.367 | 2.20E−271 |
| LOXL1 | 1.5376895 | 0.818 | 0.35 | 1.38E−269 |
| PMP22 | 1.41788255 | 0.943 | 0.518 | 1.81E−269 |
| CPE | 1.426866 | 0.959 | 0.375 | 1.86E−268 |
| SELM | 1.05873446 | 0.914 | 0.252 | 4.19E−266 |
| PLA2G2A | 1.68695957 | 0.336 | 0.041 | 2.33E−265 |
| RHOB | 1.74611814 | 0.924 | 0.497 | 1.95E−261 |
| ANXA1 | 1.25465576 | 0.995 | 0.745 | 5.76E−260 |
| MXRA7 | 1.41246367 | 0.872 | 0.451 | 3.30E−257 |
| GLUL | 1.42320425 | 0.85 | 0.383 | 7.67E−251 |
| ANXA4 | 1.31784372 | 0.815 | 0.376 | 5.93E−250 |
| VEGFB | 1.60719743 | 0.823 | 0.405 | 1.62E−249 |
| ATP5E | 1.27158738 | 0.954 | 0.35 | 1.19E−246 |
| SERPING1 | 1.33676939 | 0.973 | 0.497 | 1.73E−246 |
| ISLR | 1.17085012 | 0.759 | 0.275 | 8.19E−243 |
| ADD3 | 1.27960938 | 0.836 | 0.388 | 1.02E−242 |
| ITM2B | 1.2473098 | 0.997 | 0.864 | 1.62E−241 |
| AHNAK | 1.34149084 | 0.953 | 0.576 | 5.13E−241 |
| PPP1R14B | 1.4972506 | 0.873 | 0.477 | 3.92E−239 |
| EMILIN2 | 1.05051822 | 0.601 | 0.182 | 8.76E−236 |
| KLF2 | 1.46273277 | 0.85 | 0.375 | 2.90E−233 |
| DUSP1 | 1.52796025 | 0.965 | 0.612 | 3.60E−229 |
| CYBRD1 | 1.2159414 | 0.859 | 0.431 | 8.11E−228 |
| FOS | 1.66308847 | 0.969 | 0.704 | 4.24E−227 |
| CEBPD | 1.66209849 | 0.882 | 0.459 | 2.66E−226 |
| VAT1 | 1.17478031 | 0.718 | 0.298 | 5.18E−223 |
| TGFBR2 | 1.11772815 | 0.731 | 0.281 | 4.38E−222 |
| MMP2 | 1.23276799 | 0.926 | 0.486 | 7.22E−221 |
| OGN | 1.14443346 | 0.846 | 0.279 | 6.02E−220 |
| SDCBP | 1.27030017 | 0.939 | 0.593 | 8.68E−218 |
| LAPTM4A | 1.01707001 | 0.995 | 0.845 | 3.41E−214 |
| RAB32 | 1.25795032 | 0.866 | 0.439 | 6.02E−214 |
| GLIPR2 | 1.21714498 | 0.805 | 0.397 | 2.77E−211 |
| MT-ND4L | 1.23725862 | 0.951 | 0.648 | 1.74E−210 |
| FCGRT | 1.04193618 | 0.869 | 0.446 | 3.86E−204 |
| MYL12B | 1.01756222 | 0.976 | 0.749 | 3.42E−203 |
| TSPAN4 | 1.0819279 | 0.811 | 0.368 | 2.05E−202 |
| JUND | 1.34944873 | 0.959 | 0.638 | 1.43E−201 |
| PTHLH | 1.36803283 | 0.524 | 0.157 | 2.03E−200 |
| SFRP4 | 1.61269784 | 0.697 | 0.255 | 4.20E−198 |
| SERPINF1 | 1.11340658 | 0.958 | 0.523 | 7.41E−198 |
| DBI | 1.11676819 | 0.919 | 0.596 | 1.35E−196 |
| RNH1 | 1.10814177 | 0.924 | 0.581 | 1.00E−191 |
| C3 | 1.00241644 | 0.468 | 0.109 | 2.00E−190 |
| ACTG1 | 1.06065964 | 0.984 | 0.843 | 4.26E−189 |
| RHOA | 1.01432309 | 0.961 | 0.692 | 7.04E−183 |
| UGDH | 1.07185698 | 0.714 | 0.331 | 1.88E−179 |
| LTBP4 | 1.01481366 | 0.957 | 0.452 | 3.37E−179 |
| CYB5A | 1.00604521 | 0.716 | 0.324 | 3.22E−176 |
| COPZ2 | 1.06996532 | 0.701 | 0.328 | 7.56E−174 |
| DPYSL3 | 1.02506208 | 0.669 | 0.296 | 1.16E−173 |
| SH3BGRL3 | 1.19477133 | 0.978 | 0.682 | 3.93E−172 |
| RRAS | 1.11599092 | 0.78 | 0.441 | 4.35E−163 |
| ZFP36L2 | 1.10967884 | 0.912 | 0.504 | 8.94E−163 |
| TAF10 | 1.00772919 | 0.686 | 0.322 | 7.24E−161 |
| UGP2 | 1.23150287 | 0.788 | 0.457 | 3.42E−157 |
| SSR3 | 1.03218628 | 0.895 | 0.583 | 4.11E−150 |
| IER2 | 1.24167016 | 0.889 | 0.587 | 8.04E−133 |
| EGR1 | 1.01658508 | 0.862 | 0.528 | 3.52E−127 |
| YWHAH | 1.00573712 | 0.742 | 0.477 | 2.80E−106 |

TABLE 2E
Genes for detection in fine needle aspiration (FNA) biopsy sample

| ACTA2_CAF_Genes | avg_log2FC | pct. 1 | pct. 2 | p_val_adj |
|---|---|---|---|---|
| TINAGL1 | 1.71653124 | 0.702 | 0.157 | 0 |
| S100A4 | 1.46647473 | 0.945 | 0.591 | 0 |
| FRZB | 1.90429509 | 0.653 | 0.162 | 0 |
| PTMA | 1.09671962 | 0.994 | 0.966 | 0 |
| SOD3 | 1.95612764 | 0.779 | 0.355 | 0 |
| IGFBP7 | 1.21717408 | 0.999 | 0.845 | 0 |
| SPARCL1 | 1.58155926 | 0.954 | 0.447 | 0 |
| CALD1 | 1.31869988 | 0.947 | 0.903 | 0 |
| TPM2 | 2.30906266 | 0.938 | 0.589 | 0 |
| TAGLN | 2.91027761 | 0.99 | 0.519 | 0 |
| ADIRF | 2.7497607 | 0.965 | 0.424 | 0 |
| ACTA2 | 3.1514551 | 0.975 | 0.547 | 0 |
| CRIP1 | 2.26962961 | 0.907 | 0.565 | 0 |
| TPM1 | 1.64044012 | 0.88 | 0.739 | 0 |
| MYH11 | 2.34523075 | 0.473 | 0.024 | 0 |
| DSTN | 2.07518157 | 0.963 | 0.837 | 0 |
| MYL9 | 2.62803496 | 0.965 | 0.747 | 0 |
| PPP1R14A | 1.70423794 | 0.733 | 0.349 | 0 |
| BCAM | 1.75308281 | 0.407 | 0.083 | 0 |
| PLN | 1.93072208 | 0.43 | 0.056 | 0 |
| WFDC1 | 2.12089688 | 0.438 | 0.019 | 0 |
| RERGL | 1.76191212 | 0.189 | 0.003 | 0 |
| ESAM | 1.35182732 | 0.384 | 0.081 | 1.18E−299 |
| SEPT4 | 1.45672125 | 0.503 | 0.144 | 1.30E−293 |
| PGF | 1.64785798 | 0.607 | 0.232 | 1.44E−284 |
| MEF2C | 1.3927966 | 0.658 | 0.245 | 7.95E−281 |
| MYLK | 1.7932663 | 0.621 | 0.273 | 1.34E−273 |
| LGI4 | 1.09513594 | 0.262 | 0.042 | 1.56E−267 |
| CALM2 | 1.18497268 | 0.945 | 0.871 | 2.30E−266 |
| COX4I2 | 1.18917685 | 0.586 | 0.186 | 1.06E−261 |
| LMOD1 | 1.02786874 | 0.251 | 0.04 | 3.92E−258 |
| PTP4A3 | 1.56907151 | 0.471 | 0.151 | 1.44E−248 |
| ID3 | 1.32385186 | 0.811 | 0.461 | 1.13E−246 |
| MCAM | 1.67091233 | 0.586 | 0.272 | 3.83E−231 |
| NDUFA4L2 | 1.00468193 | 0.882 | 0.523 | 4.95E−216 |
| CDH6 | 1.11171092 | 0.385 | 0.107 | 1.93E−201 |
| HIGD1B | 1.0262334 | 0.505 | 0.165 | 2.62E−197 |
| GNB2L1 | 1.09259863 | 0.73 | 0.347 | 1.79E−196 |
| RCAN2 | 1.32394326 | 0.33 | 0.093 | 6.68E−196 |
| MFGE8 | 1.52745782 | 0.705 | 0.483 | 3.03E−195 |
| HSPB1 | 1.33733613 | 0.902 | 0.83 | 2.84E−190 |
| SEPW1 | 1.25762095 | 0.574 | 0.245 | 6.39E−186 |
| LBH | 1.6252613 | 0.496 | 0.221 | 3.80E−185 |
| IFITM2 | 1.1878277 | 0.802 | 0.687 | 6.20E−184 |
| PRKCDBP | 1.36553927 | 0.508 | 0.203 | 1.90E−183 |
| ATP5I | 1.14936312 | 0.604 | 0.271 | 6.42E−179 |
| NTRK2 | 1.00860164 | 0.268 | 0.063 | 1.01E−177 |
| ATP5G2 | 1.09918291 | 0.608 | 0.275 | 1.44E−176 |
| JUNB | 1.5000598 | 0.797 | 0.69 | 2.53E−175 |
| ATP5L | 1.04942026 | 0.628 | 0.293 | 1.45E−168 |
| SORBS2 | 1.55038697 | 0.337 | 0.115 | 5.35E−162 |
| TPPP3 | 1.10027025 | 0.633 | 0.317 | 7.52E−154 |
| PHLDA2 | 1.79825012 | 0.521 | 0.293 | 1.58E−151 |
| GPRC5C | 1.09652818 | 0.319 | 0.102 | 4.60E−146 |
| PTRF | 1.18832793 | 0.44 | 0.179 | 6.01E−146 |
| USMG5 | 1.03620917 | 0.563 | 0.262 | 1.88E−145 |
| MAP3K7CL | 1.31237174 | 0.325 | 0.111 | 1.41E−143 |
| RGS16 | 1.42294848 | 0.336 | 0.11 | 1.77E−142 |
| HES4 | 1.42029738 | 0.606 | 0.411 | 2.25E−139 |
| UQCR11.1 | 1.06560485 | 0.262 | 0.073 | 5.90E−133 |
| ID1 | 1.39874884 | 0.432 | 0.201 | 4.08E−127 |
| TBX2 | 1.10452956 | 0.322 | 0.115 | 1.82E−123 |
| C14orf2 | 1.00020157 | 0.525 | 0.251 | 2.56E−122 |
| CRYAB | 1.28064491 | 0.562 | 0.345 | 4.36E−122 |
| MGST3 | 1.05100091 | 0.772 | 0.754 | 8.55E−122 |
| OAZ2 | 1.4735048 | 0.604 | 0.464 | 1.98E−116 |
| CSRP1 | 1.65233583 | 0.55 | 0.416 | 1.56E−104 |
| GPX3 | 1.04347681 | 0.39 | 0.178 | 6.74E−102 |
| LINC00152 | 1.09291753 | 0.393 | 0.173 | 1.01E−101 |
| FOS | 1.16377456 | 0.767 | 0.71 | 1.25E−98 |
| COL18A1 | 1.1048293 | 0.663 | 0.562 | 3.84E−94 |
| GADD45B | 1.56488718 | 0.609 | 0.512 | 6.21E−90 |
| CRIP2 | 1.20006638 | 0.625 | 0.546 | 7.39E−89 |
| MT2A | 1.23077383 | 0.916 | 0.838 | 2.34E−84 |
| CSRP2 | 1.47674452 | 0.493 | 0.347 | 7.81E−84 |
| C11orf96 | 1.39644288 | 0.54 | 0.413 | 4.79E−77 |
| TSC22D1 | 1.2998306 | 0.589 | 0.523 | 2.45E−73 |
| ACTG2 | 1.43458688 | 0.185 | 0.063 | 4.58E−71 |
| ID4 | 1.14737339 | 0.371 | 0.206 | 5.23E−71 |
| ZFP36 | 1.34783844 | 0.632 | 0.569 | 5.54E−67 |
| SH3BGRL | 1.03551216 | 0.634 | 0.625 | 1.07E−66 |
| EPAS1 | 1.13976913 | 0.455 | 0.317 | 1.41E−63 |
| SOCS3 | 1.26530555 | 0.405 | 0.254 | 1.80E−59 |
| MT1G | 2.04216679 | 0.184 | 0.069 | 5.35E−57 |
| MT1E | 1.45666933 | 0.702 | 0.667 | 7.15E−47 |
| PDLIM1 | 1.07933152 | 0.472 | 0.376 | 1.87E−46 |
| MT1M | 2.19174618 | 0.364 | 0.25 | 3.04E−40 |
| PDGFA | 1.10911676 | 0.352 | 0.247 | 1.60E−38 |
| NET1 | 1.29367298 | 0.313 | 0.23 | 1.95E−29 |
| MT1A | 1.28800936 | 0.162 | 0.077 | 1.38E−28 |
| MT1X | 1.90003671 | 0.533 | 0.528 | 6.03E−23 |
| CRIM1 | 1.01824039 | 0.262 | 0.186 | 7.26E−21 |
| HSPA1B | 1.26755119 | 0.468 | 0.46 | 5.27E−19 |
| CNN1 | 1.06925951 | 0.243 | 0.178 | 1.44E−15 |
| FAM107B | 1.06226394 | 0.271 | 0.215 | 2.38E−15 |
| LPP | 1.05062568 | 0.434 | 0.454 | 4.03E−15 |
| HSPA1A | 1.24073362 | 0.559 | 0.641 | 1.34E−10 |
| HSPA6 | 1.65398338 | 0.171 | 0.123 | 3.56E−07 |
| CDKN1A | 1.00528105 | 0.327 | 0.318 | 1.02E−04 |

TABLE 2F
Genes for detection in fine needle aspiration (FNA) biopsy sample

| RGS5_CAF_Genes | avg_log2FC | pct. 1 | pct. 2 | p_val_adj |
|---|---|---|---|---|
| ID3 | 2.18269938 | 0.804 | 0.417 | 0 |
| TINAGL1 | 1.7200137 | 0.538 | 0.122 | 0 |
| GJA4 | 2.00375151 | 0.458 | 0.097 | 0 |
| RGS5 | 3.29899205 | 0.987 | 0.216 | 0 |
| CALM2 | 1.16764842 | 0.901 | 0.871 | 0 |
| FRZB | 1.42397386 | 0.452 | 0.142 | 0 |
| IGFBP7 | 1.87737639 | 0.998 | 0.825 | 0 |
| SPARCL1 | 1.93021339 | 0.921 | 0.389 | 0 |
| CPE | 1.43169035 | 0.668 | 0.337 | 0 |
| CDH6 | 1.45935463 | 0.339 | 0.081 | 0 |
| MEF2C | 1.87933767 | 0.625 | 0.199 | 0 |
| SKP1 | 1.21386447 | 0.778 | 0.814 | 0 |
| FAM162B | 1.3908053 | 0.306 | 0.057 | 0 |
| STEAP4 | 1.1951541 | 0.334 | 0.065 | 0 |
| TPM2 | 1.12044873 | 0.743 | 0.586 | 0 |
| IFITM2 | 1.47510589 | 0.729 | 0.688 | 0 |
| IFITM1 | 1.60745165 | 0.659 | 0.392 | 0 |
| THY1 | 1.3069022 | 0.743 | 0.613 | 0 |
| ITGB1 | 1.10033373 | 0.796 | 0.84 | 0 |
| ADIRF | 1.68502363 | 0.839 | 0.382 | 0 |
| A2M | 1.62217068 | 0.583 | 0.167 | 0 |
| ARHGDIB | 1.95968792 | 0.631 | 0.328 | 0 |
| NDUFA4L2 | 1.56409967 | 0.912 | 0.471 | 0 |
| RPL21 | 1.27170206 | 0.98 | 0.942 | 0 |
| PGF | 1.72896097 | 0.492 | 0.208 | 0 |
| CRIP1 | 1.55622989 | 0.784 | 0.547 | 0 |
| B2M | 1.27482338 | 0.999 | 0.952 | 0 |
| TPPP3 | 2.14133152 | 0.705 | 0.261 | 0 |
| HIGD1B | 2.41830837 | 0.634 | 0.093 | 0 |
| SEPT4 | 1.48005831 | 0.391 | 0.122 | 0 |
| COX4I2 | 2.30201335 | 0.671 | 0.116 | 0 |
| MYL9 | 1.20098382 | 0.827 | 0.748 | 0 |
| ATP5G3 | 1.26473945 | 0.466 | 0.196 | 0 |
| ATP5I | 1.50145847 | 0.59 | 0.231 | 0 |
| SEPP1 | 1.34458482 | 0.491 | 0.18 | 0 |
| GNB2L1 | 1.39036515 | 0.755 | 0.293 | 0 |
| SHFM1 | 1.37694581 | 0.553 | 0.225 | 0 |
| ATP5L | 1.50986101 | 0.638 | 0.248 | 0 |
| USMG5 | 1.4712759 | 0.569 | 0.222 | 0 |
| ATP5G2 | 1.6433791 | 0.629 | 0.228 | 0 |
| LHFP | 2.14745988 | 0.694 | 0.186 | 0 |
| C14orf2 | 1.44471326 | 0.54 | 0.213 | 0 |
| TCEB2 | 1.40527837 | 0.616 | 0.247 | 0 |
| ATP5E | 1.42928257 | 0.759 | 0.288 | 0 |
| SEPW1 | 1.15899907 | 0.499 | 0.219 | 8.44E−300 |
| NGFRAP1 | 1.26796303 | 0.438 | 0.179 | 9.83E−299 |
| ATP5J2 | 1.22812329 | 0.453 | 0.194 | 7.15E−285 |
| ATP5B | 1.32610841 | 0.407 | 0.165 | 9.59E−283 |
| LPL | 1.03867356 | 0.177 | 0.029 | 2.80E−274 |
| GUCY1B3 | 1.10457969 | 0.259 | 0.068 | 5.27E−268 |
| ATP5J | 1.16776846 | 0.466 | 0.212 | 1.97E−265 |
| ATP5D | 1.20671877 | 0.44 | 0.195 | 1.48E−263 |
| GMFG | 1.2073072 | 0.294 | 0.092 | 4.88E−261 |
| ATP5O | 1.22444573 | 0.401 | 0.171 | 9.47E−254 |
| RPS4Y1 | 1.24864379 | 0.387 | 0.163 | 9.51E−236 |
| APOE | 1.25205912 | 0.624 | 0.471 | 8.33E−231 |
| CYGB | 1.75771811 | 0.517 | 0.361 | 6.73E−225 |
| FXYD6 | 1.24298522 | 0.286 | 0.103 | 1.91E−212 |
| GLTSCR2 | 1.10534403 | 0.352 | 0.151 | 1.05E−202 |
| HOPX | 1.12658112 | 0.241 | 0.08 | 3.56E−185 |
| SEP15 | 1.04195386 | 0.359 | 0.17 | 2.36E−176 |
| ENPEP | 1.02715278 | 0.237 | 0.082 | 1.84E−172 |
| COL18A1 | 1.35484113 | 0.603 | 0.563 | 1.78E−170 |
| WBP5 | 1.0344961 | 0.356 | 0.167 | 3.50E−170 |
| PTP4A3 | 1.25949974 | 0.314 | 0.144 | 6.02E−165 |
| LINC00998 | 1.01335416 | 0.21 | 0.068 | 7.76E−164 |
| PRKCDBP | 1.00321503 | 0.385 | 0.191 | 8.82E−163 |
| OLFML2A | 1.20104314 | 0.305 | 0.138 | 9.91E−162 |
| RASD1 | 1.47323657 | 0.278 | 0.119 | 3.62E−159 |
| ATP5A1 | 1.00031056 | 0.283 | 0.121 | 5.17E−153 |
| CHCHD10 | 1.41608614 | 0.486 | 0.374 | 1.48E−152 |
| ATPIF1 | 1.02713238 | 0.323 | 0.158 | 1.20E−142 |
| CHN1 | 1.55724924 | 0.45 | 0.331 | 1.94E−142 |
| CD36 | 1.33238299 | 0.241 | 0.1 | 3.38E−133 |
| NDRG2 | 1.25754367 | 0.297 | 0.156 | 9.24E−126 |
| MFGE8 | 1.17338695 | 0.542 | 0.49 | 1.55E−118 |
| KCNJ8 | 1.12865384 | 0.258 | 0.127 | 5.44E−116 |
| ARHGAP15 | 1.39495602 | 0.351 | 0.227 | 1.52E−113 |
| C20orf27 | 1.49760675 | 0.4 | 0.309 | 1.40E−101 |
| OAZ2 | 1.37264163 | 0.482 | 0.472 | 1.66E−79 |
| PLXDC1 | 1.21740193 | 0.357 | 0.267 | 7.27E−77 |
| CD9 | 1.04848251 | 0.533 | 0.518 | 1.02E−75 |
| MCAM | 1.10936202 | 0.368 | 0.279 | 3.80E−70 |
| SH3BGRL | 1.13651849 | 0.555 | 0.641 | 2.42E−68 |
| ALDOA | 1.02529979 | 0.501 | 0.501 | 5.86E−59 |
| NOTCH3 | 1.02003723 | 0.484 | 0.482 | 1.98E−51 |
| CRIP2 | 1.12961013 | 0.498 | 0.563 | 7.92E−43 |
| PDGFRB | 1.04921205 | 0.493 | 0.548 | 8.74E−39 |
| COL4A1 | 1.03318376 | 0.486 | 0.53 | 1.04E−30 |
| MYO1B | 1.20016596 | 0.36 | 0.36 | 2.28E−26 |
| SLC12A2 | 1.11228813 | 0.224 | 0.173 | 9.94E−26 |
| EBF1 | 1.00261948 | 0.342 | 0.338 | 2.18E−18 |
| FAM213A | 1.01121263 | 0.262 | 0.242 | 5.36E−14 |
| STOM | 1.07396588 | 0.389 | 0.464 | 2.14E−08 |
| CSRP2 | 1.03355863 | 0.33 | 0.363 | 1.59E−05 |
| EPHX1 | 1.04058681 | 0.34 | 0.396 | 0.0015283 |

TABLE 3
Genes for detection in fine needle aspiration (FNA) biopsy sample
Normal_Fibroblast

| | | | | |
|---|---|---|---|---|
| ACACB | CAB39L | COX4I2 | HBB | SH2D1A |
| ACSM5 | CADM3 | CTSG | HBD | SLC16A12 |
| ADAMTS5 | CCDC69 | CYP21A2 | HIGD1B | SLC19A3 |
| ADRB1 | CD14 | CYP3A5 | HRCT1 | SLC1A2 |
| AGTR1 | CD34 | CYP4B1 | IL33 | SLC2A4 |
| AKR1C4 | CD36 | CYP4F12 | KANK3 | SLC7A4 |
| ANKRD20A4P | CDH6 | CYP4X1 | KCNA2 | SLC8A1 |
| ANKRD31 | CDHR3 | DACT2 | KCNAB1 | SMOC1 |
| AQP7 | CDHR4 | DGAT2 | KLF2 | SPAG17 |
| ARHGAP6 | GDF10 | FHL5 | PPARG | TCF7L1 |
| ASPA | GHR | KRT222 | PPP1R1A | TEF |
| ATOH8 | GPD1 | LIFR | PRG2 | TNMD |
| ATP1A2 | HBA1 | LMO3 | PXDNL | TNNT3 |
| ATP8B4 | HBA2 | LRAT | REEP1 | TNXB |
| BMP5 | DGKB | LRP1B | RET | TPSAB1 |
| C1QTNF7 | DNASE1L3 | MLXIPL | S1PR1 | TPSB2 |
| FAM241A | DPY19L2 | NEGR1 | SCARA5 | TRHDE |
| CA3 | EBF2 | NOVA1 | SCARF1 | VIPR1 |
| CHRDL1 | EBF3 | NPR1 | SCN4A | VIT |
| CIDEC | ECM2 | NPY1R | SCN4B | VSIG10L |
| CLEC3B | ESAM | NTN1 | CAVIN2 | VWF |
| COL21A1 | F10 | NTN4 | SEC14L5 | WDR17 |
| FNDC5 | FAM162B | PCOLCE2 | SEMA6C | WNT11 |
| FRMPD1 | FAM180B | PGM5 | SERINC4 | ZDHHC11 |
| FZD4 | FBLN5 | PID1 | SGCA | ZNF304 |
| G0S2 | | PLIN1 | SPRR2F | ZNF839 |
| | | PLIN4 | STEAP4 | |

iCAF

| | | | | |
|---|---|---|---|---|
| CCL19 | EGFLAM | CAPN6 | LSP1 | SLIT3 |
| CRABP1 | PLXDC1 | SCARA5 | PTGS2 | HSPB6 |
| CYP1B1 | CXCL11 | DLK1 | NR2F1 | IL6 |
| TNFRSF4 | CCL8 | MEG3 | CEBPA | FBLN5 |
| CXCL13 | CXCL3 | TAC1 | KDM6B | OGN |
| CXCL9 | ITM2A | THUMPD3-AS1 | PID1 | PLA2G2A |
| IL34 | VEGFD | GMFG | ADH1B | CHRDL1 |
| ABI3BP | FXYD1 | VCAM1 | CXCL12 | CYGB |
| CYP7B1 | PLPP3 | FMO4 | TNXB | FGF7 |
| F10 | TNFRSF10D | PDGFD | RSPO3 | PI16 |
| COLEC12 | LAMC3 | GADD45G | DCN | PLAC9 |
| EMILIN1 | CXCL10 | SNAI1 | C16orf89 | CCN5 |
| GGT5 | ZBTB16 | | GDF10 | PTX3 |
| | | | PAMR1 | CCL2 |

myCAF

| | | | | |
|---|---|---|---|---|
| SFRP4 | COL8A1 | DPT | TGFB1 | FBN1 |
| CCDC80 | GAS1 | ELN | TGFB3 | MATN3 |
| OGN | COL3A1 | FBLN2 | TNN | LRRC15 |
| DCN | OMD | TMEM204 | CST2 | ISLR |
| PTGER3 | COL11A1 | SEPTIN11 | HES4 | P3H1 |
| SFRP2 | CILP | TNFAIP6 | COL10A1 | NBL1 |
| PDGFRL | NEXN | GGT5 | THBS4 | SPON1 |
| SMOC2 | ASPN | INAFM1 | NKD2 | SULF1 |
| MMP23B | RARRES2 | OLFML2B | OLFM2 | FNDC1 |
| CPXM2 | FIBIN | IGF1 | COL6A3 | CNN1 |
| COL14A1 | TMEM119 | IGF2 | LRRC17 | MIAT |
| ITGBL1 | KERA | CST1 | THY1 | CPXM1 |
| CCN5 | ID4 | LAMP5 | HTRA3 | P4HA3 |
| CILP2 | GRP | LOXL1 | ADAM12 | GXYLT2 |
| | COMP | EDNRA | PLPP4 | P3H4 |
| | | | CREB3L1 | |

CAF_S1

| | | | | |
|---|---|---|---|---|
| LAMA2 | ABI3BP | TBX5-AS1 | GAS1 | SCARA5 |
| SFRP4 | GALNT5 | CXCL12 | PTX3 | IGFL2 |
| PDGFRA | RSPO3 | HTRA3 | FLNC | CEMIP |
| LRRC15 | WTAPP1 | DIO2 | SPON1 | PI16 |
| GREM1 | PODN | TNNT3 | CST1 | FAM133CP |
| SFRP2 | CHRDL1 | COL6A3 | CNTN3 | SLC66A1L |
| JCAD | CILP | KCNK2 | COMP | AOX1 |
| DCN | PRDM6 | KERA | CD177 | CLCN4 |
| CPXM2 | CCL11 | COL11A1 | MFAP4 | HS3ST3A1 |
| PTGFR | CAPN6 | STMN2 | LSP1 | TMEM176B |
| WNT2 | PDGFRL | SYNDIG1 | F13A1 | CST2 |
| ADAMTSL1 | PTGS2 | ISLR | PLA2G2A | FBN1 |
| FBLN5 | SEMA3D | CCN5 | GDF10 | GXYLT2 |
| DPP4 | DPT | BEND6 | UST | FGL2 |
| P4HA3 | NOX4 | ADH1C | FHAD1 | GABRB2 |
| CCDC80 | DNM3 | GRIA3 | OGN | COL3A1 |
| PRICKLE1 | COL6A6 | FAT4 | LIPG | SLC1A7 |
| GALNT12 | CPZ | FNDC1 | SORCS2 | |
| ZFHX4 | FLRT2 | COL10A1 | FGF7 | |
| EPYC | NEGR1 | ADH1B | ABCA9 | |
| DCHS1 | BMPER | OMD | | |

CAF_S1

| | | | | |
|---|---|---|---|---|
| MMP2 | SPOCK1 | COMP | TMEM176B | SPON2 |
| MFAP5 | EPYC | F2R | CST2 | CPEB1 |
| LAMA2 | FBLN1 | CD177 | FBN1 | ARHGAP28 |
| SFRP4 | MXRA5 | MBP | GXYLT2 | PLXDC2 |
| LUM | DCHS1 | TNFSF4 | FGL2 | CLIC6 |
| PDGFRA | QPCT | MFAP4 | SERPINE1 | TNXB |
| LRRC15 | DNM3 | LSP1 | GABRB2 | TIMP2 |
| GREM1 | HTRA1 | F13A1 | ALDH1A3 | KLK4 |
| SFRP2 | COL6A6 | PLA2G2A | VEPH1 | CADPS |
| JCAD | CPZ | LSAMP | COL3A1 | IRS2 |
| DCN | SNCAIP | GDF10 | MILR1 | CTSB |
| THBS2 | FLRT2 | PTX3 | ACSL5 | BHLHE22 |
| CPXM2 | NPR3 | BDKRB1 | HS3ST3A1 | ZFHX4-AS1 |
| PTGFR | PMP22 | FLNC | SLC1A7 | ABCA10 |
| WNT2 | SOD2 | MMP1 | PLXNC1 | MEG3 |
| ADAMTSL1 | LPAR1 | SPON1 | S100A10 | NTM |
| CTSK | FAM20A | CST1 | SERPINE2 | C1R |
| GJB2 | NEGR1 | CNTN3 | DKK2 | ARL4C |
| BHMT2 | BNC2 | UST | PLPP4 | DRP2 |
| GAS7 | BMPER | FHAD1 | CXCL13 | FIBIN |
| CTHRC1 | TBX5-AS1 | OGN | F2RL2 | SIGLEC17P |
| C3 | FOXP2 | LIPG | SLC24A2 | FGD6 |
| FBLN5 | CXCL12 | SORCS2 | MEG8 | PDGFD |
| DPP4 | HTRA3 | SCG2 | GFPT2 | SRPX |
| P4HA3 | DIO2 | FRMD6 | C14orf132 | FKBP9P1 |
| CCDC80 | JAM2 | FGF7 | NAV1 | SLC6A6 |
| PTGIS | TNNT3 | ABCA9 | RNF144A | SAT1 |
| PRICKLE1 | COL6A3 | CCN5 | SSC5D | ADAM12 |
| C1S | MIR100HG | LRP1 | HAS2 | UAP1 |
| GALNT12 | KCNK2 | PLAUR | TENM1 | COL15A1 |
| ABI3BP | MOXD1 | BEND6 | FAM155A | PKD1L2 |
| GALNT15 | KERA | ADH1C | ST3GAL5 | SLC7A8 |
| RSPO3 | COL11A1 | DEPDC7 | HSPA12B | IGFBP6 |
| WTAPP1 | STMN2 | GRIA3 | PRTFDC1 | RSPO1 |
| PODN | SYNDIG1 | MFAP2 | GPNMB | MIR3120 |
| CHRDL1 | FAP | RARRES1 | EFEMP1 | IGF1 |
| CILP | ELMO1 | FAT4 | SRPX2 | FSTL1 |
| PRDM6 | LRRC2 | FNDC1 | LINC00922 | APOD |
| PTGDS | ISLR | SCARA5 | KIAA0930 | ADAMTS16 |
| ABCA6 | ALDH1A1 | IGFL2 | SVEP1 | PRELP |
| CCL11 | COL10A1 | CEMIP | PDPN | VCAN |
| CAPN6 | SNED1 | PI16 | DNM1 | CYP11A1 |
| PDGFRL | CDH11 | PTGFRN | TDRD10 | GPR68 |
| DAB2 | XG | FAM133CP | BICC1 | SLC1A3 |
| PTGS2 | SIRPA | SLC66A1L | AHNAK2 | MMP11 |
| SEMA3D | ADH1B | AOX1 | MAPK7 | CLIC2 |
| EMP1 | OMD | CLCN4 | FGFR1 | COL4A3 |
| DPT | POSTN | CHL1 | ACKR4 | GPC3 |
| NOX4 | MMP13 | ADAM23 | CELF2 | PCSK5 |
| ZFHX4 | GAS1 | HMCN1 | LPAR4 | TCN2 |

CAF_S1_Subset_IFNγ-iCAF_{—}

| | | | | |
|---|---|---|---|---|
| CCL19 | TNFRSF4 | ABI3BP | CXCL10 | PLXDC1 |
| VCAM1 | CXCL13 | CYP7B1 | EMILIN1 | CXCL11 |
| CRABP1 | CXCL9 | F10 | GGT5 | CCL8 |
| CYP1B1 | CCL2 | COLEC12 | EGFLAM | CXCL3 |
| CXCL10 | IL34 | | | |

CAF_S1_Subset_wound-myCAF

| | | | | |
|---|---|---|---|---|
| SFRP4 | SMOC2 | COL8A1 | ASPN | COMP |
| CCDC80 | MMP23B | GAS1 | RARRES2 | DPT |
| OGN | CPXM2 | COL3A1 | FIBIN | ELN |
| DCN | COL14A1 | OMD | TMEM119 | FBLN2 |
| PTGER3 | ITGBL1 | COL11A1 | KERA | IGF1 |
| SFRP2 | CCN5 | CILP | ID4 | IGF2 |
| PDGFRL | CILP2 | NEXN | GRP | |

CAF S1_Subset_TGFβ-myCAF

| | | | | |
|---|---|---|---|---|
| CST1 | TNN | NKD2 | HTRA3 | ID4 |
| LAMP5 | CST2 | OLFM2 | TMEM204 | GGT5 |
| LOXL1 | HES4 | COL6A3 | SEPTIN11 | INAFM1 |
| EDNRA | COL10A1 | LRRC17 | COMP | CILP |
| TGFB1 | ELN | COL3A1 | TNFAIP6 | OLFML2B |
| TGFB3 | THBS4 | THY1 | | |

CAF_S1_Subset_IL-iCAF

| | | | | |
|---|---|---|---|---|
| ITM2A | CXCL10 | THUMPD3-AS1 | COLEC12 | PTGS2 |
| CXCL12 | ZBTB16 | GMFG | PDGFD | NR2F1 |
| VEGFD | CAPN6 | CYGB | GADD45G | F10 |
| FXYD1 | SCARA5 | CCL8 | SNAI1 | CEBPA |
| PLPP3 | DLK1 | VCAM1 | LSP1 | KDM6B |
| TNFRSF10D | MEG3 | FMO4 | CCL2 | PID1 |
| LAMC3 | TAC1 | FBLN5 | IL6 | |

CAF_S1_Subset_detox-iCAF

| | | | | |
|---|---|---|---|---|
| ADH1B | C16orf89 | SLIT3 | PLA2G2A | PLAC9 |
| CXCL10 | GDF10 | HSPB6 | CHRDL1 | CCN5 |
| CXCL12 | PAMR1 | IL6 | CYGB | PTX3 |
| TNXB | FXYD1 | FBLN5 | FGF7 | CCL2 |
| RSPO3 | ZBTB16 | OGN | PI16 | CXCL3 |
| DCN | | | | |

CAF_S1_Subset_ecm-myCAF

| | | | | |
|---|---|---|---|---|
| ASPN | ITGBL1 | FBN1 | SEPTIN11 | CPXM1 |
| COL3A1 | COL8A1 | LOXL1 | NBL1 | FIBIN |
| THY1 | COL14A1 | MATN3 | SPON1 | P4HA3 |
| SFRP2 | ADAM12 | LRRC15 | SULF1 | GXYLT2 |
| COL10A1 | OLFML2B | COMP | FNDC1 | CILP2 |
| COL6A3 | ELN | ISLR | CNN1 | P3H4 |
| LRRC17 | PLPP4 | P3H1 | MIAT | CCDC80 |
| CILP | CREB3L1 | COL11A1 | MMP23B | |
| GRP | | | | |

iCAF

| | | | | |
|---|---|---|---|---|
| C3 | SRPX | THBS1 | FSTL1 | IGFBP6 |
| DUSP1 | MT2A | CCL2 | FBLN2 | FBN1 |
| FBLN1 | MEDAG | OGN | NR4A3 | BDKRB1 |
| LMNA | IGF1 | GSN | MFAP5 | TPPP3 |
| CLU | MGST1 | DPT | ABL2 | RASD1 |
| CCDC80 | MCL1 | PLA2G2A | SGK1 | MT1A |
| MYC | CEBPD | NAMPT | CILP | CXCL14 |
| EFEMP1 | S100A10 | ITM2A | UGDH | PI16 |
| HAS1 | UAP1 | RGCC | FBLN5 | APOE |
| NR4A1 | TNXB | JUND | ADAMTS1 | CXCL8 |
| CFD | CEBPB | NNMT | ADH1B | ARC |
| ANXA1 | PNRC1 | ZFP36 | CCN5 | PTX3 |
| CXCL12 | SOCS3 | PIM1 | GPX3 | TNFAIP6 |
| FGF7 | PTGDS | CPE | S100A4 | MT1E |
| KLF4 | FOSB | GFPT2 | IL6 | MT1X |
| EMP1 | NFKBIA | SOD2 | HAS2 | CXCL1 |
| GPRC5A | CXCL2 | KDM6B | PLAC9 | |

myCAF

| | | | | |
|---|---|---|---|---|
| MYL9 | BGN | CTHRC1 | INHBA | POSTN |
| CALD1 | IGFBP7 | ACTA2 | COL10A1 | GRP |
| MMP11 | TPM2 | TAGLN | TPM1 | CST1 |
| HOPX | | | | |

pan-myCAF

| | | | | |
|---|---|---|---|---|
| ADIRF | TPM2 | CSRP1 | CPM | CRYAB |
| ACTA2 | PTP4A3 | CAV1 | PGF | OLFML2A |
| MYH11 | PPP1R14A | ADAMTS4 | GUCY1B1 | TIMP3 |
| TAGLN | CRIP2 | GJA4 | UBA2 | GUCY1A1 |
| SPARCL1 | ADAMTS1 | RGS5 | YIF1A | FILIP1 |
| MCAM | CSRP2 | MEF2C | PHLDA1 | FAM13C |
| A2M | NDUFA4L2 | CALM2 | NDRG2 | NDUFS4 |
| MYLK | TPM1 | APOLD1 | ID3 | ITGB1 |
| IGFBP7 | MAP1B | OAZ2 | RGS16 | KCNE4 |
| CRIP1 | FRZB | MGST3 | CYB5R3 | CPE |
| TINAGL1 | CAVIN3 | ISYNA1 | | |

pan-dCAF

| | | | | |
|---|---|---|---|---|
| COL1A1 | CTSK | VCAN | ANTXR1 | PLAU |
| THBS2 | INHBA | SULF1 | CPXM1 | MORF4L2 |
| CTHRC1 | TNFAIP6 | IGFBP3 | COL6A1 | UAP1 |
| COL3A1 | ADAM12 | COL8A1 | ASPN | SERPINF1 |
| LUM | THY1 | GREM1 | PDLIM4 | ITGB1 |
| COL1A2 | FN1 | DCN | ITGA11 | TGFBI |
| LGALS1 | STEAP1 | ITGA5 | PRSS23 | HTRA3 |
| COL5A1 | SPON2 | RIN2 | COL6A2 | C1R |
| POSTN | PLAUR | TMEM119 | SFRP2 | TIMP1 |
| SERPINE1 | SPHK1 | TNFRSF12A | YIF1A | LMNA |
| LOXL2 | LOX | P4HA3 | SNAI2 | CYP1B1 |
| COL11A1 | EMP1 | CRABP2 | C1S | MGP |
| COL12A1 | ANGPTL2 | TPM4 | TMEM176B | ANGPTL4 |
| MMP2 | RARRES2 | LOXL1 | CCN2 | |

pan-iCAF

| | | | | |
|---|---|---|---|---|
| CFD | CXCL12 | SFRP2 | TMEM176B | SLC40A1 |
| GPC3 | ABI3BP | PTGDS | SERPINF1 | FHL2 |
| C3 | FBLN1 | DCN | FHL1 | ELN |
| ADH1B | MGST1 | MGP | GPX3 | KLF4 |
| IGF1 | MFAP4 | C1S | CTGF | RARRES1 |
| EFEMP1 | PLA2G2A | IGFBP6 | C1R | CCN1 |
| PODN | DPT | GSN | SFRP4 | IGFBP5 |
| SELENOP | WISP2 | TMEM176A | CYP1B1 | |
| | CCDC80 | FIBIN | CST3 | |

pan-iCAF-2

| | | | | |
|---|---|---|---|---|
| IER3 | SOD2 | GEM | GFPT2 | EGR1 |
| CXCL2 | FOSB | NR4A3 | JUNB | ABI3BP |
| ICAM1 | PIM1 | APOD | THBS1 | GADD45B |
| TNFAIP2 | ZFP36 | SAT1 | CDKN1A | DUSP1 |
| NFKBIA | CLU | UAP1 | C3 | RARRES1 |
| NR4A1 | ABL2 | OGN | CYP1B1 | CST3 |
| CCL2 | | | | |

pan-pCAF

| | | | | |
|---|---|---|---|---|
| NUSAP1 | ADAM12 | THY1 | CTHRC1 | COL8A1 |
| DIAPH3 | LOX | CD248 | COL5A1 | COL6A1 |
| LOXL2 | POSTN | FN1 | LOXL1 | COL6A2 |
| | | COL12A1 | COL1A1 | |

TABLE 4
Genes for detection in fine needle aspiration (FNA) biopsy sample

| | | | |
|---|---|---|---|
| gene symbol | avg_log2FC | gene symbol | avg_log2FC |

SELENOP+ Macro

| | | | |
|---|---|---|---|
| SELENOP | 4.1 | SERPING1 | 1.46 |
| PLTP | 2.98 | FUCA1 | 1.42 |
| C1QB | 2.68 | MRC1 | 1.4 |
| FOLR2 | 2.58 | CFD | 1.39 |
| CCL18 | 2.57 | TSPAN4 | 1.32 |
| RNASE1 | 2.56 | PSAP | 1.32 |
| C1QA | 2.53 | CREG1 | 1.26 |
| C1QC | 2.45 | CTSZ | 1.25 |
| SLC40A1 | 2.4 | CTSB | 1.24 |
| MS4A4A | 2.06 | FCGRT | 1.24 |
| MMP12 | 2.04 | SDC3 | 1.24 |
| CXCL9 | 1.97 | A2M | 1.21 |
| APOE | 1.94 | IFI27 | 1.17 |
| LGMN | 1.92 | BLVRB | 1.16 |
| F13A1 | 1.88 | GPNMB | 1.16 |
| IL2RA | 1.83 | MAF | 1.15 |
| CD14 | 1.83 | CD163 | 1.13 |
| CCL13 | 1.78 | TMEM176A | 1.13 |
| PLD3 | 1.77 | SLCO2B1 | 1.13 |
| STAB1 | 1.76 | NINJ1 | 1.08 |
| C2 | 1.62 | MS4A7 | 1.07 |
| DAB2 | 1.62 | MS4A6A | 1.06 |
| PDK4 | 1.58 | CD68 | 1.05 |
| CTSC | 1.55 | CD209 | 1.03 |
| TMEM176B | 1.49 | VAMP5 | 1 |
| VSIG4 | 1.48 | FCGR1A | 1 |

SPP1+ MARCO+ Macro

| | | | |
|---|---|---|---|
| CCL18 | 2.76 | CD68 | 1.35 |
| APOC1 | 2.76 | CTSB | 1.31 |
| CSTB | 2.57 | PRDX1 | 1.28 |
| NUPR1 | 2.48 | GCHFR | 1.27 |
| GPNMB | 2.38 | IFI27 | 1.23 |
| FTL | 2.32 | MMP9 | 1.22 |
| CTSD | 2.27 | BRI3 | 1.22 |
| SPP1 | 2.23 | CD52 | 1.18 |
| MARCO | 2.18 | PPDPF | 1.18 |
| CTSL | 2.02 | MT1X | 1.16 |
| FABP5 | 2.01 | CD63 | 1.14 |
| FBP1 | 1.94 | ATOX1 | 1.09 |
| LGALS3 | 1.93 | PLIN2 | 1.08 |
| ACP5 | 1.73 | PSAP | 1.08 |
| APOE | 1.69 | CTSZ | 1.07 |
| MT2A | 1.57 | BLVRB | 1.07 |
| TXN | 1.5 | MT1E | 1.06 |
| FTH1 | 1.47 | CCL2 | 1.06 |
| HMOX1 | 1.46 | GSTO1 | 1.04 |
| LIPA | 1.45 | MIF | 1.02 |
| CYP27A1 | 1.38 | ANXA2 | 1.02 |

SPP1+ TGFBI+ Macro

| | | | |
|---|---|---|---|
| SPP1 | 2.21 | IFI27 | 1.29 |
| RNASE1 | 2.21 | STAB1 | 1.22 |
| CTSL | 1.77 | IL7R | 1.22 |
| CTSD | 1.67 | APOC1 | 1.2 |
| APOE | 1.62 | MMP9 | 1.18 |
| ISG15 | 1.55 | MTRNR2L8 | 1.14 |
| CTSB | 1.54 | PLD3 | 1.12 |
| IFI6 | 1.54 | CXCL3 | 1.09 |
| SDS | 1.41 | PSAP | 1.09 |
| LGMN | 1.4 | ACP5 | 1.06 |
| GPNMB | 1.32 | FABP5 | 1.06 |
| TGFBI | 1.32 | LY6E | 1.01 |

CD1C+ cDC2

| | | | |
|---|---|---|---|
| CCL17 | 2.52 | RGCC | 1.17 |
| FCER1A | 2.02 | PKIB | 1.11 |
| AREG | 1.83 | HLA-DQA1 | 1.04 |
| CD1C | 1.52 | S100B | 1.02 |

CD1C+ CD1A+ cDC2

| | | | |
|---|---|---|---|
| S100B | 4.33 | IL22RA2 | 1.34 |
| HLA-DQB2 | 3.16 | GSN | 1.29 |
| LTB | 3.12 | NDRG2 | 1.26 |
| CD1A | 2.65 | LST1 | 1.26 |
| CD207 | 2.01 | LMNA | 1.17 |
| CD1E | 1.99 | CST7 | 1.17 |
| FCER1A | 1.9 | HLA-DQA2 | 1.16 |
| TACSTD2 | 1.88 | CST3 | 1.14 |
| CD1C | 1.82 | PLEK2 | 1.1 |
| FCGBP | 1.72 | VASP | 1.08 |
| C15orf48 | 1.67 | CD52 | 1.06 |
| PKIB | 1.64 | PPA1 | 1.02 |
| RUNX3 | 1.59 | | |

TABLE 5
Genes for detection in fine needle aspiration (FNA) biopsy sample

| [symbol] | p_val [symbol] | avg_log2FC [symbol] | pct. 1 [symbol] | pct. 2 [symbol] | p_val_adj [symbol] |
|---|---|---|---|---|---|
| SFRP2 | 0.000000e+00 | 3.5664549 | 0.920 | 0.163 | 0.000000e+00 |
| POSTN | 4.770352e−241 | 3.2330781 | 0.715 | 0.219 | 1.706975e−236 |
| CTHRC1 | 0.000000e+00 | 3.1959111 | 0.907 | 0.294 | 0.000000e+00 |
| COL1A1 | [symbol] .417015e−293 | 3. [symbol] 288641 | 0.989 | 0. [symbol] 04 | 3.011659e−288 |
| COMP | 0.000000e+00 | 2.9507268 | 0.386 | 0.016 | 0.000000e+00 |
| VCAN | 0.000000e+00 | 2.7928497 | 0.572 | 0.239 | 0.000000e+00 |
| LUM | 0.000000e+00 | 2.7810478 | 0.984 | 0.464 | 0.000000e+00 |
| COL1A2 | 1.082965e−275 | 2. [symbol] 528355 | 0.994 | 0.784 | 3.875184e−271 |
| COL3A1 | 2.961 [symbol] 99e−256 | 2.4834787 | 0.990 | 0.723 | 1. [symbol] 59506e−251 |
| COL11A1 | 0.000000e+00 | 2.4395897 | 0.448 | 0.040 | 0.000000e+00 |
| SFRP4 | 0.000000e+00 | 2.3987069 | 0.745 | 0.146 | 0.000000e+00 |
| COL10A1 | 0.000000e+00 | 2.3985988 | 0.531 | 0.044 | 0.000000e+00 |
| COL8A1 | 0.000000e+00 | 2.3573200 | 0.581 | 0.067 | 0.000000e+00 |
| HTRA1 | 1.240707e−187 | 2.2495763 | 0.746 | 0.308 | 4.439620e−183 |
| ASPN | 4.330942e−151 | 2.2005413 | 0.750 | 0.317 | 1.342741e−146 |
| THBS2 | 9.298321e−279 | 2.1769243 | 0.727 | 0.190 | 3.327218e−274 |
| CXCL14 | 3.013208e−87 | 2.1614847 | 0.344 | 0.118 | 1.078932e−62 |
| ITGBL1 | 7.512429e−271 | 2.1407549 | 0.515 | 0.090 | 2.668172e−266 |
| CST1 | 4.535774e−167 | 2.1237001 | 0.100 | 0.002 | 1.623036e−182 |
| IGF1 | 9.922805e−128 | 2.0651463 | 0.348 | 0.074 | 3.550677e−123 |
| NBL1 | 3.378246e−179 | 2.0516962 | 0.756 | 0.347 | 1.208638e−174 |
| CTGF | 1.277602e−96 | 2.0435825 | 0.656 | 0.351 | 4.571645e−92 |
| AEBP1 | 1.787160e−191 | 1.9695618 | 0. [symbol] | 0.410 | 6.394994e−187 |
| MMP11 | 2.160323e−60 | 1.9680989 | 0.373 | 0.146 | 7.730283e−56 |
| IGFBP3 | 5.285289e−236 | 1.9639341 | 0.372 | 0.049 | 1.891235e−231 |
| FN1 | 6.504999e−128 | 1.9077475 | 0.922 | 0.693 | 2.327684e−123 |
| CTSK | 7.835334e−134 | 1.8765108 | 0.718 | 0.278 | 2.803717e−179 |
| CDH11 | 3.417492e−242 | 1.8133271 | 0.611 | 0.150 | 1.222881e−237 |
| INHBA | 1. [symbol] 26651e−199 | 1.8030246 | 0.545 | 0.130 | 6.894134e−195 |
| MMP2 | 8.571431e−193 | 1.7277582 | 0.707 | 0.219 | 3.087115e−1 [symbol] |
| SULF1 | 2.231504e−134 | 1.6889804 | 0.992 | 0.219 | 7.984991e−130 |
| COL5A2 | 1.593643e−136 | 1.63 [symbol] 88 | 0.799 | 0.431 | 3.702531e−132 |
| COL5A2 | 1.593643e−136 | 1.6339588 | 0.799 | 0.431 | 3.702531e−132 |
| ISLR | 2.824790e−149 | 1.6088390 | 0.595 | 0.208 | 9.392284e−145 |
| COL6A3 | 4.413489e−160 | 1.3814515 | 0.896 | 0.316 | 1.579279e−155 |
| COL5A1 | 2.714362e−125 | 1.3783043 | 0.676 | 0.304 | 9.712 [symbol] 2e−121 |
| MXRA5 | 5.197751e−134 | 1.5709322 | 0.560 | 0.197 | 1.859911e−129 |
| FAP | 9.435911e−192 | 1.5313222 | 0.337 | 0.143 | 3.376452e−187 |
| SPARC | 2.314821e−154 | 1.4 [symbol] 2019 | 0.986 | 0.879 | 8.283522e−150 |
| LAMP5 | 2.636231e−181 | 1.4391616 | 0.366 | 0.068 | 9.433226e−177 |
| CYP181 | 2.720438e−110 | 1.4343444 | 0.495 | 0.165 | 9.734544e−108 |
| DPT | 1.492439e− [symbol] | 1.4313713 | 0.348 | 0.120 | 5.340394e−37 |
| ELN | 8. [symbol] 24148e−83 | 1.4245211 | 0.411 | 0.135 | 3.085979e−80 |
| DCN | 1.392232e−183 | 1.4042060 | 0.971 | 0.339 | 4.981824e−179 |
| RARRES2 | 1.271735e−87 | 1.379834 [symbol] | 0.671 | 0.370 | 4.550649e−83 |
| SDC1 | 0.000000e+00 | 1.3753397 | 0.293 | 0.017 | 0.000000e+00 |
| OMD | 1.454268e−147 | 1.3395107 | 0.434 | 0.101 | 5.203507e−143 |
| COL12A1 | 5.705976e−43 | 1.3362720 | 0.501 | 0.286 | 2.041770e−33 |
| PRELP | 1.769290e−48 | 1.3301037 | 0.410 | 0.194 | 6.331051e−44 |
| CCDC80 | 4.898663e−103 | 1.3141155 | 0.737 | 0.385 | 1.781060e−57 |
| MMP14 | 2.972888e−83 | 1.3120329 | 0.541 | 0.229 | 1.063766e−78 |
| MDK | 9.627136e−81 | 1.3037888 | 0.695 | 0.361 | 3.444878e−76 |
| MARCKS | 1.910407e−91 | 1.2968890 | 0.754 | 0.486 | 6.835987e−87 |
| LTBP2 | 2.439694e−120 | 1.2786622 | 0.469 | 0.144 | 8.729958e−116 |
| ANTXR1 | 2.760838e−66 | 1.2594710 | 0.577 | 0.319 | 9.379106e−62 |
| TGM2 | 4.010619e−126 | 1.2552527 | 0.289 | 0.054 | 1.435120e−121 |
| ANKH | 7.559413e−160 | 1.2492971 | 0.481 | 0.109 | 2.704985e−155 |
| C18 | 1.354656e−114 | 1.2401457 | 0.770 | 0.399 | 4.847365e−110 |
| OGN | 1.174423e−64 | 1.2357548 | 0.373 | 0.131 | 4.202439e−60 |
| GJA1 | 7.110855e−102 | 1.2356788 | 0.416 | 0.123 | 2.344477e−97 |
| MFAP2 | 1.354124e−111 | 1.2307024 | 0.340 | 0.081 | 4.845463e−107 |
| SERPINE2 | 3.045918e−55 | 1.2275286 | 0.469 | 0.215 | 1.089921e−50 |
| FAM1980 | 1.328899e−132 | 1.2181490 | 0.362 | 0.080 | 4.754340e−123 |
| PTGDS | 4.232702e−35 | 1.1692926 | 0.169 | 0.052 | 1.314588e−30 |
| PTGDS | 4.232702e−35 | 1.1692926 | 0.169 | 0.052 | 1.314588e−30 |
| PPIC | 1.879073e−104 | 1.1586615 | 0.729 | 0.360 | 6.723887e−100 |
| F2R | 1.042505e−73 | 1.1521810 | 0.372 | 0.128 | 3.750397e−69 |
| MMP13 | 2.646621e−112 | 1.1504356 | 0.110 | 0.008 | 9.470404e−108 |
| NNMT | 3.081169e−73 | 1.1389538 | 0.713 | 0.430 | 1.102535e−70 |
| PRSS23 | 6.367690e−57 | 1.1317895 | 0.684 | 0.436 | 2.278550e−52 |
| SPON2 | 4.280881e−61 | 1.1278705 | 0.493 | 0.227 | 1.531709e−56 |
| PLXDC2 | 6.551944e−105 | 1.1098197 | 0.463 | 0.148 | 2.344462e−100 |
| FBLN1 | 8.457128e−139 | 1.1023287 | 0.796 | 0.343 | 5.028214e−134 |
| TMEM119 | 1.444242e−162 | 1.0993676 | 0.387 | 0.068 | 5.167930e−158 |
| TIMP3 | 9.493705e−24 | 1.0789979 | 0.700 | 0.566 | 3.397132e−19 |
| RAB31 | 1.609701e−50 | 1.0734707 | 0.553 | 0.317 | 3.759992e−46 |
| ITGB5 | 1.901894e−88 | 1.0698913 | 0.494 | 0.196 | 6.805548e−84 |
| LOXL1 | 1.011556e−92 | 1.0646420 | 0.440 | 0.147 | 3.619578e−88 |
| DIO2 | 9.904488e−66 | 1.0617049 | 0.343 | 0.116 | 3.344123e−61 |
| DPYSL3 | 1.683544e−124 | 1.0558392 | 0.440 | 0.121 | 6.024227e−120 |
| RCN3 | 3.306876e−80 | 1.0440284 | 0.539 | 0.240 | 1.234863e−75 |
| FGF7 | 9.134435e−59 | 1.0334984 | 0.477 | 0.207 | 3.288993e−54 |
| EDIL3 | 5.697170e−94 | 1.0289283 | 0.375 | 0.107 | 2.0 [symbol] 616e−89 |
| TTC3 | 7.298371e−34 | 1.0250988 | 0.393 | 0.355 | 2.611576e−49 |
| COL16A1 | 6.668758e−93 | 1.0220272 | 0.470 | 0.151 | 2.386282e−90 |
| SERPINF1 | 1.850614e−59 | 1.0178480 | 0.762 | 0.578 | 6.622052e−55 |
| SERPINE1 | 8.785796e−51 | 1.0120093 | 0.345 | 0.133 | 3.145821e−45 |
| FNDC1 | 1.872554e−129 | 1.0094493 | 0.332 | 0.068 | 6.700555e−125 |
| SUGCT | 2.603583e−116 | 1.0066330 | 0.238 | 0.038 | 9.316400e−112 |
| TNBS1 | 3. [symbol] 18150e−30 | 1.0049740 | 0.410 | 0.229 | 1.294688e−29 |
| CERCAM | 3.743395e−90 | 1.0006300 | 0.450 | 0.138 | 2.055945e−85 |

[symbol]

indicates data missing or illegible when filed

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are described herein.

In certain instances, genes, nucleotides, and polypeptides disclosed herein are included in publicly-available databases. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, in some embodiments±0.1%, in some embodiments±0.01%, and in some embodiments±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The terms “diagnosing” and “diagnosis” as used herein, such as in the context of a high risk of presence, metastasis, and/or recurrence of thyroid cancer, refer to methods by which the skilled artisan can estimate and even determine whether or not a subject is suffering from a given disease or condition. The skilled artisan often makes a diagnosis on the basis of one or more diagnostic indicators, which is indicative of the presence, severity, or absence of the condition.

Along with diagnosis, clinical disease prognosis is also an area of great concern and interest. It is important to know the stage and rapidity of advancement of a cancer in order to plan the most effective therapy. If a more accurate prognosis can be made, appropriate therapy, and in some instances less severe therapy for the patient can be chosen.

As such, “making a diagnosis” or “diagnosing”, as used herein, is further inclusive of determining a prognosis, which can provide for predicting a clinical outcome (with or without medical treatment), selecting an appropriate treatment (or whether treatment would be effective), or monitoring a current treatment and potentially changing the treatment, based on the measure of gene expression levels disclosed herein.

The phrase “determining a prognosis” as used herein refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the presence, absence or levels of test biomarkers. Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, the chance of a given outcome may be about 3%. In certain embodiments, a prognosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, or about a 95% chance.

The skilled artisan will understand that associating a prognostic indicator with a predisposition to an adverse outcome is a statistical analysis. Preferred confidence intervals of the present subject matter are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

In other embodiments, a threshold degree of change in the level of a prognostic or diagnostic indicator can be established, and the degree of change in the level of the indicator in a biological sample can simply be compared to the threshold degree of change in the level. In yet other embodiments, a “nomogram” can be established, by which a level of a prognostic or diagnostic indicator can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

The terms “correlated” and “correlating,” as used herein in reference to the use of diagnostic and prognostic biomarkers, refers to comparing the presence or quantity of the biomarkers in a subject to its presence or quantity in subjects known to suffer from, or known to be at risk of, a given condition (e.g., a cancer); or in subjects known to be free of a given condition, i.e. “normal individuals.”

With respect to the presently-disclosed subject matter, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided in accordance with the presently-disclosed subject matter. As such, the presently-disclosed subject matter provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The presently-disclosed subject matter is further illustrated by the following specific but non-limiting examples. The following examples may include compilations of data that are representative of data gathered at various times during the course of development and experimentation related to the present invention.

### EXAMPLES

### Example 1—Mutational Landscape of Aggressive Thyroid Cancer

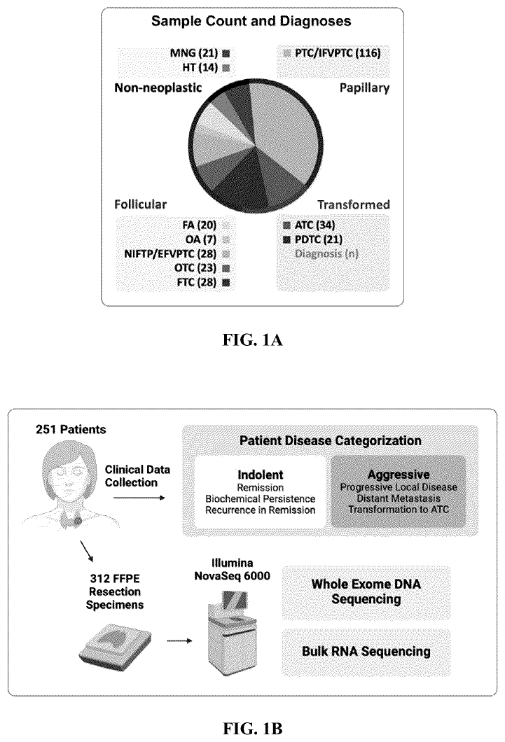

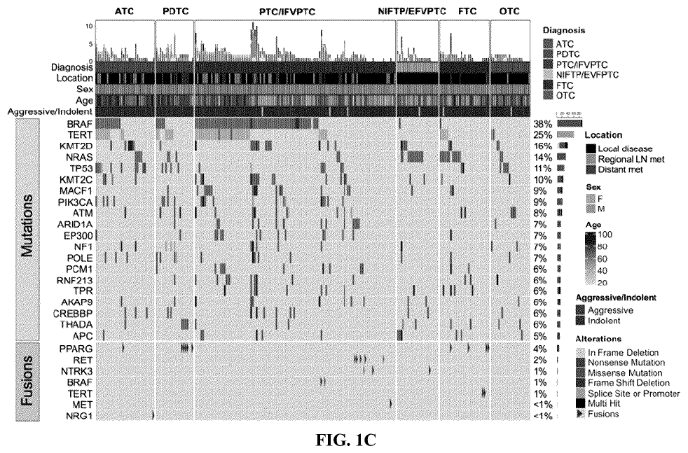

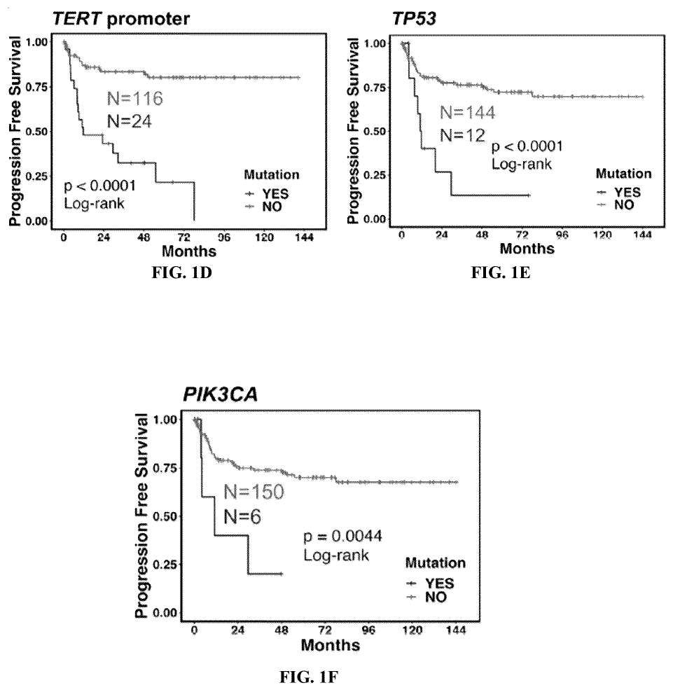

To identify gene mutations and fusions associated with aggressive thyroid cancer, we performed whole-exome and bulk RNA sequencing on 312 formalin-fixed paraffin-embedded (FFPE) resection samples from 251 patients with thyroid nodules, including non-neoplastic, neoplastic, and malignant lesions (FIG. 1A-1B) from two tertiary care centers. The cohort was enriched for aggressive lesions to include well-differentiated tumors with distant metastases and transformed thyroid cancers (ATCs and PDTCs). Samples from age-matched patients with well-differentiated cancers without distant metastases and patients with benign thyroid lesions were collected between the same date range (FIG. 1A, Table 6). We next categorized patients as having either “indolent” or “aggressive” disease (FIG. 1B, detailed definitions in STAR Methods). Sequencing analyses identified common driver thyroid cancer alterations at frequencies comparable to those previously reported (FIG. 1C, Table 7).^{10,11,12,13,27}

TABLE 6
Thyroid cohort patient and lesion characteristics.

| | | | Patients with | | | | Patients with well- | | Patients with | |
|---|---|---|---|---|---|---|---|---|---|---|
| | | | non-neoplastic | | Patients with | | differentiated | | transformed | |
| | All patients | | pathology | | benign neoplasms | | malignancies | | tumors | |
| | (n = 251 patients) | | (n = 35 patients) | | (n = 27 patients) | | (n = 150 patients) | | (n = 39 patients) | |
| Age at initial surgery | 53.8 | (41.1-66.1) | 53.0 | (42.5-61.9) | 55.0 | (45.6-67.0) | 50.3 | (35.3-63.2) | 66.8 | (61.2-75.6) |
| in years, median (IQR) | | | | | | | | | | |
| Sex, female, n (%) | 163 | (64%) | 27 | (77%) | 19 | (70%) | 97 | (65%) | 20 | (51%) |
| Race, white, n (%) | 217 | (87%) | 31 | (89%) | 25 | (93%) | 126 | (84%) | 35 | (90%) |
| History of radiation, n (%) | 31 | (12%) | 2 | (6%) | 2 | (7%) | 21 | (14%) | 6 | (15%) |
| Family history of | 13 | (5%) | 4 | (11%) | 2 | (7%) | 7 | (5%) | 0 | (0%) |
| thyroid cancer, n (%) | | | | | | | | | | |

| Non-metastatic only, n (%) | 157 | (63%) | — | — | 84 | (56%) | 11 | (28%) |
|---|---|---|---|---|---|---|---|---|
| Local metastasis, n (%) | 75 | (30%) | — | — | 53 | (35%) | 22 | (56%) |
| Distant metastasis, n (%) | 56 | (22%) | — | — | 34 | (23%) | 22 | (56%) |
| Average length of | 54.3 | | — | — | 60 | | 33.3 | |
| follow-up in months | | | | | | | | |
| Average PFS length in months | 40.3 | | — | — | 48 | | 12.4 | |
| Aggressive disease, n (%) | 74 | (30%) | — | — | 38 | (25%) | 36 | (92%) |
| Death from disease, n (%) | 29 | (12%) | — | — | 8 | (53%) | 21 | (54%) |

Patient characteristics (rows) by all patients and by subgroup from least to most advanced disease subgroup from left to right (columns). For patients with more than one sample, the patient was assigned to column based on their most advanced sample subgroup. Patients with both local and distant metastasis are counted once in both rows. Non-neoplastic lesions include Multinodular Goiter (MNG) and Hashimoto's Thyroiditis (HT). Benign neoplasms include Follicular Adenoma (FA) and Oncocytic Adenoma (OA). Well-differentiated lesions include Noninvasive Follicular Thyroid Neoplasm with Papillary-like nuclear features (NIFTP), Follicular Thyroid Carcinoma (FTC), Oncocytic Thyroid Carcinoma (OTC), Encapsulated Follicular Variant Papillary Thyroid Carcinoma (EFVPTC), Infiltrative Follicular Variant Papillary Thyroid Carcinoma (IFVPTC), and Papillary Thyroid Carcinoma (PTC), as well as PTC with HT background. Transformed tumors include Anaplastic Thyroid Carcinoma (ATC) and Poorly Differentiated Thyroid Carcinoma (PDTC).

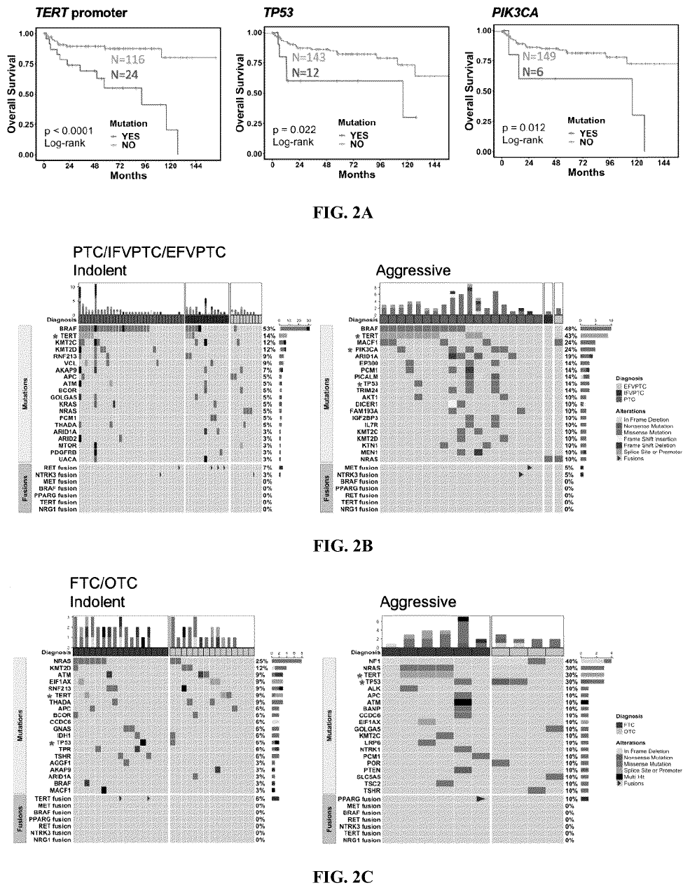

The frequency of TERTp, TP53, and PIK3CA mutations in the cohort, as they are known to be associated with distant metastasis²⁷ and transformed thyroid cancers such as PDTC and ATC.^{39,40,41} Benign lesions were negative for these high-risk mutations, as expected (Table 7). TP53 mutations were most common in ATC tumors (27%). TERTp mutations were identified in patients categorized with “aggressive” disease across histologic subtypes: PDTC (33%), ATC (31%), FTC (20%), infiltrative follicular variant papillary thyroid carcinoma (IFVPTC, 14%), and PTC (40%). PIK3CA mutations were seen in PTC (9%), PDTC (19%), and ATC (12%, Table 7). Kaplan-Meier curves confirmed that TERTp, TP53, and PIK3CA mutations were all significantly correlated with shorter progression-free survival (PFS) and overall survival (FIGS. 1D-1F and FIGS. 2A-2C). Notably, ˜42% of well-differentiated tumor samples from patients with aggressive disease lacked mutations in TERTp, TP53, or PIK3CA. Taken together, our mutational analyses indicate that while TERTp, TP53, and PIK3CA are significantly associated with aggressive disease, there is still a significant proportion of clinically aggressive thyroid cancers that lack these common high-risk mutations.

TABLE 7
Summary of key mutations.

| | | | TERT | | | |
|---|---|---|---|---|---|---|
| | BRAF | RAS | promoter | TP53 | PIK3CA | CTNNB1 |
| Diagnosis | mutation | mutation | mutation | mutation | mutation | mutation |
| MNG | 0% (0/21) | 0% (0/21) | 0% (0/19) | 0% (0/21) | 0% (0/21) | 0% (0/21) |
| HT | 0% (0/14) | 0% (0/14) | 0% (0/14) | 0% (0/14) | 0% (0/14) | 0% (0/14) |
| FA | 0% (0/20) | 15% (3/20) | 0% (0/20) | 0% (0/20) | 0% (0/20) | 0% (0/20) |
| OA | 0% (0/7) | 0% (0/7) | 0% (0/7) | 0% (0/7) | 0% (0/7) | 0% (0/7) |
| NIFTP | 0% (0/15) | 40% (6/15) | 0% (0/12) | 0% (0/15) | 0% (0/15) | 0% (0/15) |
| FTC | 4% (1/28) | 40% (11/28) | 20% (5/25) | 7% (2/28) | 0% (0/28) | 0% (0/28) |
| OTC | 0% (0/22) | 5% (1/22) | 5% (1/22) | 18% (4/22) | 0% (0/22) | 0% (0/22) |
| EFVPTC | 17% (2/12) | 42% (5/12) | 0% (0/10) | 0% (0/12) | 0% (0/13) | 0% (0/13) |
| IFVPTC | 33% (6/18) | 17% (3/18) | 14% (2/14) | 0% (0/18) | 0% (0/18) | 0% (0/18) |
| PTC | 64% (63/98) | 0% (0/98) | 40% (32/80) | 5% (5/98) | 9% (9/98) | 1% (1/98) |
| PDTC | 24% (5/21) | 14% (3/21) | 33% (7/21) | 19% (4/21) | 19% (4/21) | 5% (1/21) |
| ATC | 41% (14/34) | 12% (4/34) | 31% (10/32) | 27% (9/34) | 12% (4/34) | 0% (0/34) |

Relationship of thyroid lesion subtypes with BRAF, RAS, TERT promoter, TP53, and PIK3CA mutational status. Thyroid lesion subtype ordered by behavior from least to most aggressive. Sanger sequencing was used for TERT promoter identification (n = 276), and whole exome sequencing was used for BRAF and TP53 mutation identification (n = 310). Abbreviations: MNG = Multinodular Goiter; HT = Hashimoto Thyroiditis; FA = Follicular Adenoma; OA = Oncocytic Adenoma; NIFTP = Noninvasive Follicular Thyroid Neoplasm with Papillary-like Nuclear Features; FTC = Follicular Thyroid Carcinoma; OTC = Oncocytic Thyroid Carcinoma; EFVPTC = Encapsulated Follicular Variant Papillary Thyroid Carcinoma, IFVPTC = Infiltrative Follicular Variant Papillary Thyroid Carcinoma, PTC = Papillary Thyroid Carcinoma; PDTC = Poorly Differentiated Thyroid Carcinoma; ATC = Anaplastic Thyroid Carcinoma.

### Example 2—Molecular Aggression and Prediction (MAP) Score

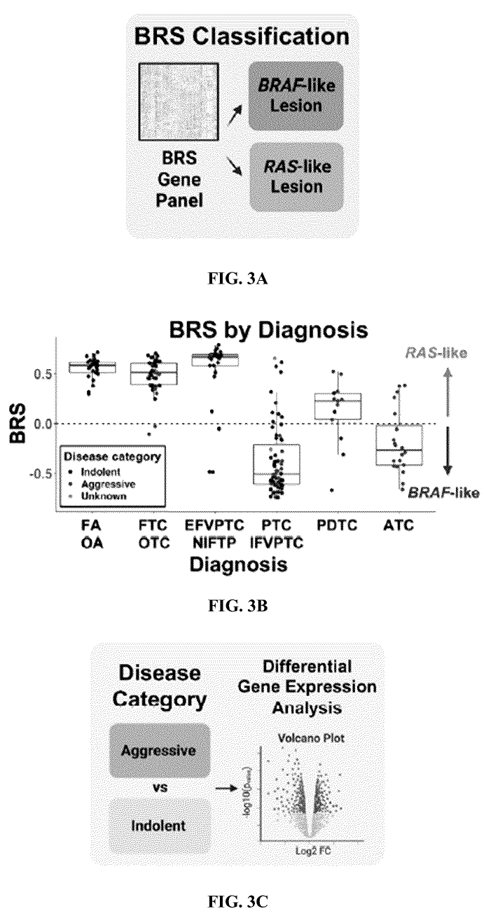

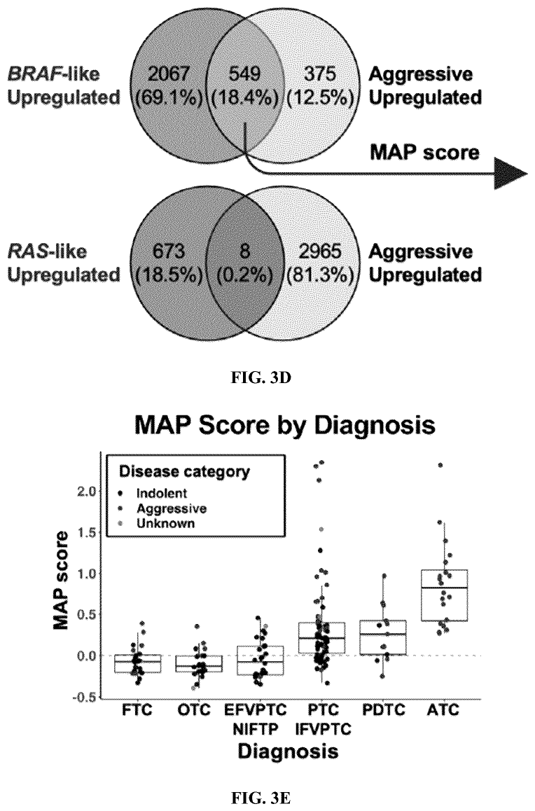

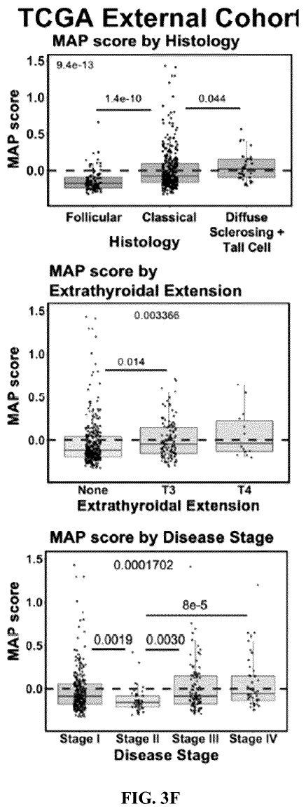

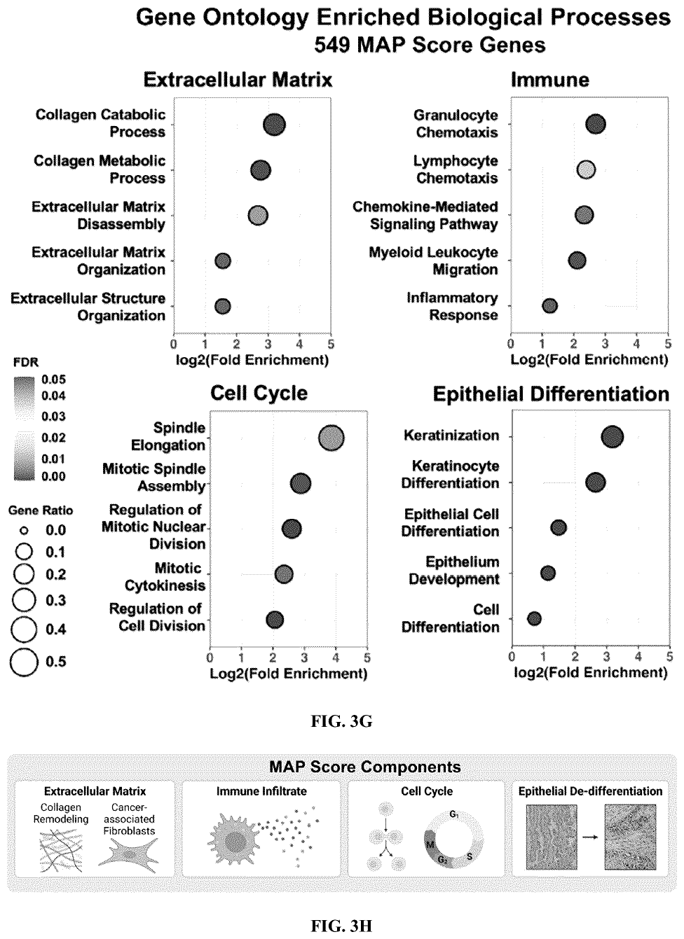

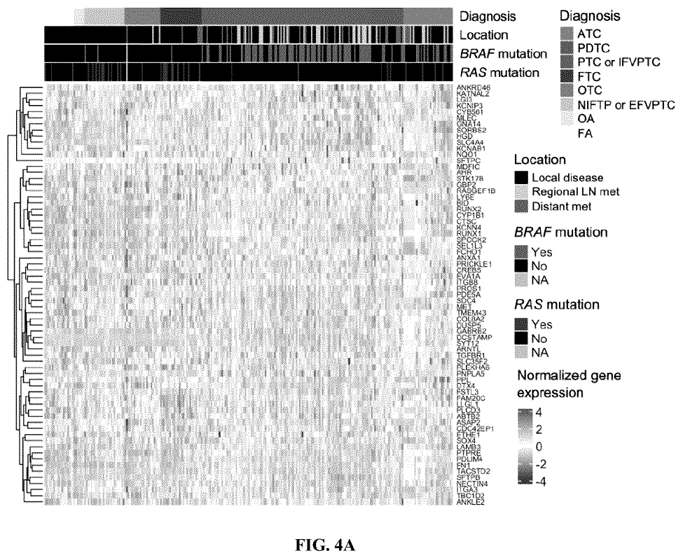

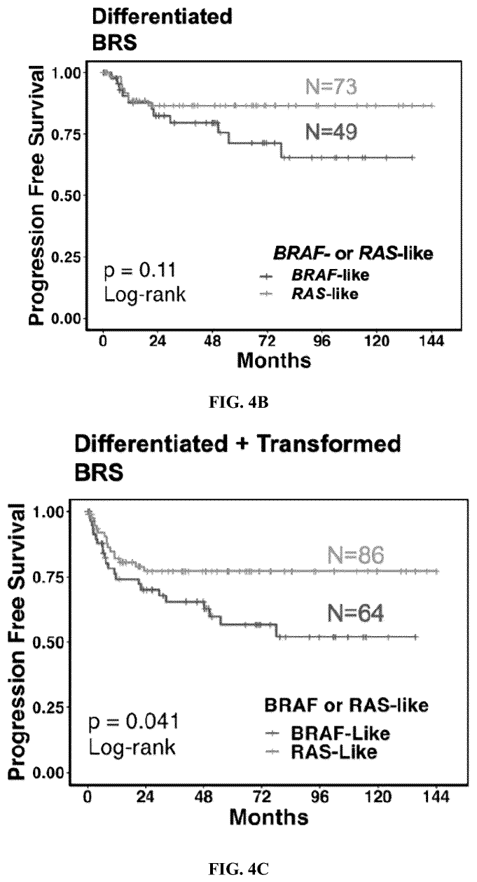

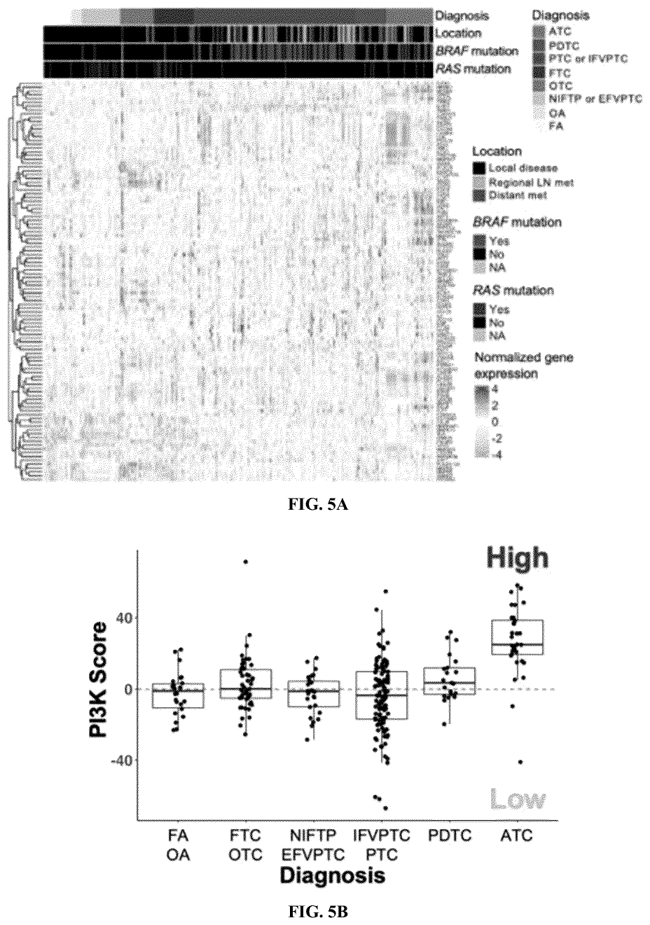

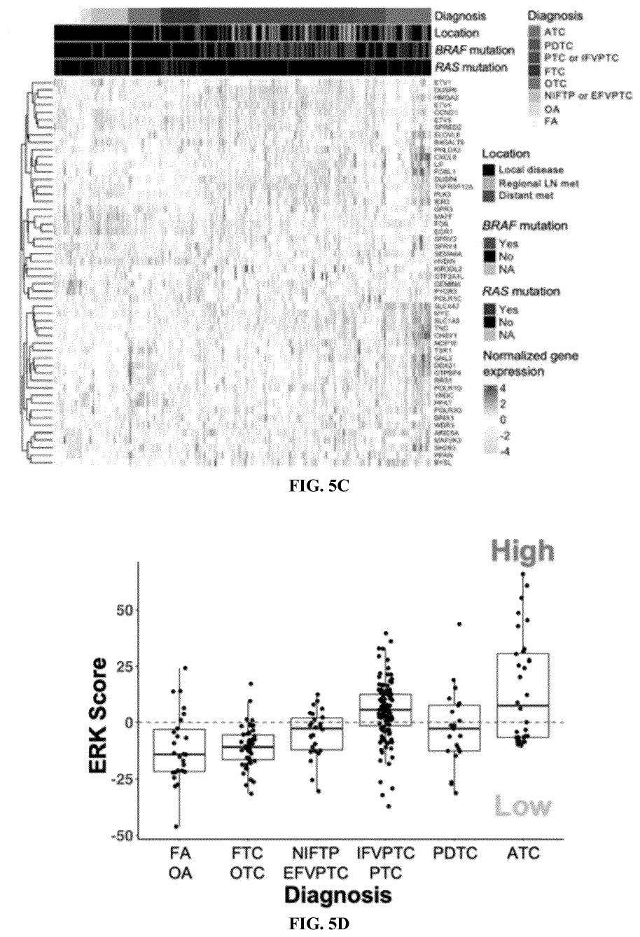

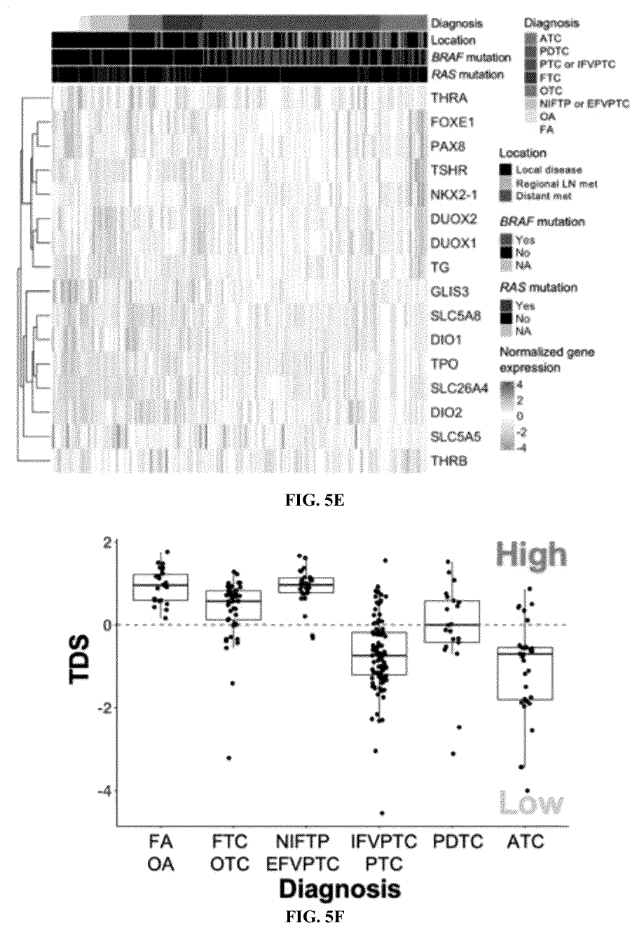

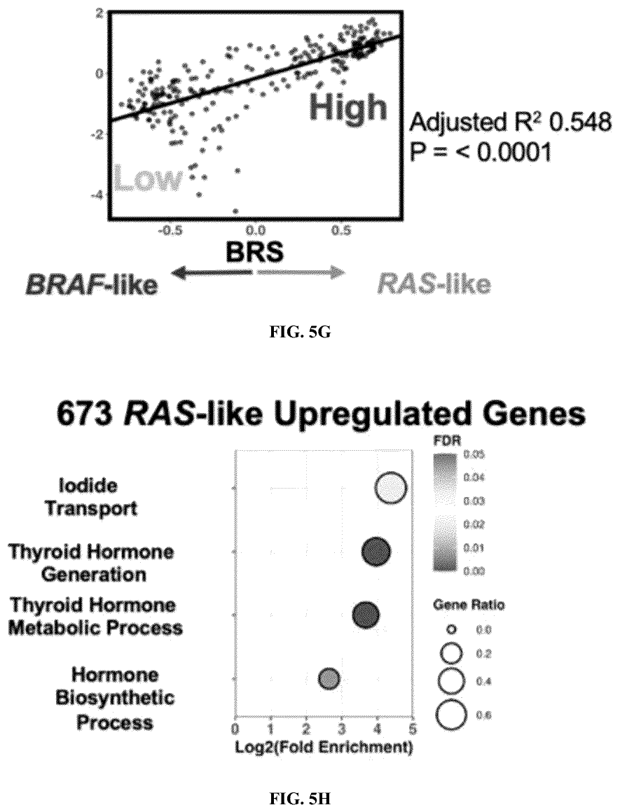

Next, previously identified gene expression signatures were explored to assess their association with aggressive disease (FIGS. 3A-3H, FIGS. 4A-4C, and FIGS. 5A-5H). Since BRAF mutational status is associated with slightly worse outcomes in some studies,20 we began by calculating the BRAF-RAS score (BRS) for each tumor, per The Cancer Genome Atlas (TCGA) classification (FIGS. 3A-3B and FIG. 4A). Approximately half of our aggressive disease samples were BRAF-like (53%). However, for well-differentiated cancer samples, BRAF-like status alone was not significantly associated with worse PFS (FIG. 4B). When transformed tumors (PDTC and ATC) were included, BRAF-like status was associated with shorter PFS, consistent with highly lethal ATCs being predominantly BRAF-like (FIG. 4C). MAPK and PI3K signaling pathways, known to be upregulated in BRAF-like and RAS-like tumors with aggressive disease, were upregulated in transformed tumors. Thyroid differentiation genes were downregulated in transformed tumors and correlated with BRAF-like status, as expected (FIG. 5A-5H). While these signatures correlated with worse survival of transformed tumors, they did not significantly predict aggressive disease in well-differentiated tumors.

To discover unique gene signatures associated with outcome, differential gene expression analysis was performed on tumors from patients categorized with either indolent or aggressive disease (FIG. 3C). As well-differentiated tumors with BRAF-like gene expression have slightly worse prognosis, we also performed differential gene expression analysis on BRAF-like or RAS-like tumors. When comparing upregulates genes in BRAF-like and aggressive disease samples, we observed a greater gene overlap in aggressive and BRAF-like samples than with RAS-like samples (549 genes versus 8 genes, FIG. 3D; Table 8). To further discriminate genes associated with both BRAF-like tumors and aggressive disease, we created a gene expression signature using the 549 genes upregulated in both aggressive and BRAF-like samples, termed the Molecular Aggression and Prediction (MAP) score. We compared this score across tumors from different histologic subtypes in our cohort. Positive MAP scores (>0) were seen for all ATCs, the majority of PDTCs, and a portion of well-differentiated thyroid cancers, predominantly from patients with aggressive disease (FIG. 3E). We further validated the MAP score on a large external cohort of well-differentiated PTCs within TCGA. Positive MAP score correlated with aggressive PTC histologic variants, such as tall cell and diffuse sclerosing, as well as adverse pathologic features such as extrathyroidal extension and advanced disease stage (FIG. 3F).

Gene ontology analysis of the MAP score genes was then used to better understand the biologic processes unique to these aggressive MAP-positive tumors. MAP-positive cancers showed enrichment of biological processes including extracellular matrix, immune-related processes, epithelial differentiation, and cell cycle processes (FIG. 3G-3H) in contrast to enrichment of thyroid metabolic processes seen only in RAS-like tumors (FIG. 5H). Epithelial de-differentiation and increased mitotic activity are known features associated with thyroid cancer progression and part of the current diagnostic criteria for many aggressive thyroid tumors, providing further support for the gene ontology analysis. 42,43,44 Altogether, the MAP score correlated with aggressive thyroid cancer subtypes and represented multiple biologic processes notably including remodeling of the tumor microenvironment.

TABLE 8
RAS- and BRAF-Aggression Overlap Genes.
RAS-Aggression Overlap Genes

| | | | | | | | |
|---|---|---|---|---|---|---|---|
| BRS3 | EEF1A2 | ISL1 | NRSN1 | OR4D5 | OR5H2 | OR6T1 | OR10S1 |

BRAF-Aggression Overlap Genes

| | | | | | | | |
|---|---|---|---|---|---|---|---|
| ACOD1 | ACP7 | ACTA1 | ACTC1 | ACY3 | ADAM19 | ADAM2 | ADAM29 |
| ADAM7 | ADAMTS14 | ADGRF4 | AFP | AGXT | AICDA | AIM2 | ALDH3B2 |
| ALPK2 | ANKRD1 | ANLN | ANPEP | ANXA8 | ANXA8L1 | APCDD1L | AQP9 |
| ARL14EPL | ARL4C | ARNTL2 | ARPP21 | ARSI | ASB10 | ASCL4 | ASPM |
| ATP12A | ATP1A4 | AURKA | AURKB | AVP | AZGP1 | B4GALNT4 | BATF |
| BCAS1 | BEND4 | BEST3 | BIRC5 | BPIFC | BUB1 | C10orf71 | C12orf75 |
| C4orf17 | C5orf46 | C9 | CA9 | CALB2 | CALHM6 | CALML3 | CBLN2 |
| CCBE1 | CCDC185 | CCDC197 | CCKAR | CCL1 | CCL11 | CCL13 | CCL20 |
| CCL24 | CCNA2 | CD19 | CD2 | CD300E | CD3E | CD7 | CD70 |
| CDC20 | CDC25C | CDCA2 | CDCA5 | CDCA8 | CDHR1 | CDK5R2 | CDX2 |
| CEACAM3 | CEACAM7 | CENPA | CENPE | CENPF | CENPI | CEP55 | CER1 |
| CHAT | CHRNA1 | CIB3 | CKAP2L | CKM | CLC | CLCA2 | CLDN14 |
| CLEC4C | CLEC4M | CLVS2 | CNGA2 | COL11A1 | COL1A1 | COL7A1 | CORO1A |
| COX6A2 | CPA4 | CPB1 | CPN2 | CPNE7 | CR1 | CR2 | CREG2 |
| CRYBA2 | CSF2 | CSF3 | CSN1S1 | CSRP3 | CST8 | CXCL1 | CXCL13 |
| CXCL3 | CXCL5 | CXCL6 | CXCL8 | CYP11B1 | CYP27C1 | CYP4F2 | CYP4F22 |
| DAZL | DDN | DEFB119 | DEFB121 | DEPDC1 | DIAPH3 | DKK1 | DLGAP5 |
| DNAJC12 | DNER | DNMT3L | DRD2 | DRGX | DSC3 | DSCAM | DSG1 |
| DSG3 | DUSP9 | DYNAP | E2F7 | E2F8 | EREG | ERICH3 | ESPL1 |
| EXD1 | EXO1 | FAM216B | FAM83A | FAM83D | FAM9A | FAM9B | FANCD2OS |
| FCAMR | FCRL3 | FCRLA | FGF5 | FOXA3 | FOXD1 | FOXE3 | FOXG1 |
| FOXI1 | FOXR2 | FPR2 | FSD1 | FST | FXYD3 | GABRA3 | GADL1 |
| GALNTL5 | GC | GDNF | GFRAL | GJA8 | GJB2 | GOLGA6L1 | GOLGA6L22 |
| GOLGA6L6 | GOT1L1 | GPR153 | GPRIN1 | GREM1 | GSDMA | GSDMC | GTSE1 |
| GTSF1L | H1-5 | H2AC16 | H2AC4 | H2BC17 | H3C12 | H3C2 | H3C3 |
| HAS2 | HCAR2 | HCAR3 | HDGFL1 | HES2 | HJURP | HMMR | HMX3 |
| HNF4G | HOXB9 | HOXC10 | HOXD10 | HOXD11 | HOXD12 | HS3ST3A1 | HTN3 |
| HTR3A | HTR7 | IBSP | IFNA8 | IFNL1 | IGF2BP1 | IL11 | IL19 |
| IL1A | IL1F10 | IL2 | IL21 | IL24 | IL2RA | IL3 | IL31RA |
| IL36B | IL36G | IQCJ | IQGAP3 | IVL | IZUMO3 | KIAA1549L | KIF14 |
| KIF15 | KIF18B | KIF20A | KIF23 | KIF4A | KIFC1 | KLF18 | KLK5 |
| KLK6 | KLK8 | KRT1 | KRT13 | KRT14 | KRT15 | KRT16 | KRT17 |
| KRT31 | KRT33A | KRT36 | KRT4 | KRT5 | KRT6A | KRT6C | KRT74 |
| KRT75 | KRT79 | KRTAP2-3 | KRTAP3-2 | KRTAP4-7 | KRTAP7-1 | L1CAM | LACTBL1 |
| LAIR2 | LAMA3 | LBP | LCE1B | LCE2A | LCE5A | LCE6A | LGALS14 |
| LHX1 | LIM2 | LORICRIN | LPAR3 | LRRC30 | LRRC38 | LY6D | LY6L |
| LYPD3 | LYPD6B | LYPD8 | MAGEA10 | MAGEA11 | MAGEA3 | MAGEB2 | MB |
| MC5R | MCHR1 | MDFIC2 | MELK | MEPE | MKI67 | MME | MMP1 |
| MMP10 | MMP11 | MMP12 | MMP13 | MMP27 | MMP3 | MMP7 | MSLNL |
| MUC7 | MUCL1 | MYBL2 | MYBPC1 | MYBPC2 | MYEOV | MYF6 | MYH13 |
| MYH2 | MYH7 | MYH8 | MYL1 | MYL2 | MYO18B | MYO1G | MYOD1 |
| MYOG | MYPN | NCAN | NCAPG | NCAPH | NDC80 | NEIL3 | NEK2 |
| NGB | NIPAL4 | NLRP10 | NLRP4 | NLRP5 | NPHS1 | NPHS2 | NRAP |
| NT5DC4 | NTNG1 | NTRK1 | NXPH2 | NYX | OBP2A | OOSP1 | OOSP2 |
| OPN1LW | OPRPN | OR10A3 | OR10W1 | OR12D2 | OR14J1 | OR1D2 | OR4A5 |
| OR4C11 | OR4D2 | OR4D9 | OR4F15 | OR4F17 | OR4K2 | OR4N2 | OR52A1 |
| OR52M1 | OR52N5 | OR52R1 | OR5AK2 | OR5BS1P | OR5H1 | OR5K4 | OR5P2 |
| OR6C1 | OR6K6 | OR8B4 | OR8G5 | OR8U3 | OTOP2 | OTP | OTX2 |
| P2RX5 | PADI6 | PAEP | PAGE2 | PAGE4 | PAQR9 | PAX5 | PBK |
| PDPN | PGLYRP3 | PI15 | PI3 | PITX1 | PITX2 | PKP1 | PLA2G2F |
| PLEKHS1 | PLPP4 | PMAIP1 | POLQ | POSTN | POTEF | POU4F2 | PRAME |
| PRAMEF1 | PRAMEF14 | PRAMEF18 | PRAMEF2 | PRAMEF20 | PRAMEF9 | PRDM13 | PRDM8 |
| PRDM9 | PRG3 | PRL | PRODH2 | PRSS21 | PRSS3 | PRSS56 | PTHLH |
| PTPRN | PTPRZ1 | PTX3 | RAB3B | RDH8 | REG3A | RESP18 | RFPL4AL1 |
| RGS21 | RGS7 | RHOV | RNASE3 | ROS1 | RRM2 | RTBDN | RTL3 |
| S100A2 | S100A7 | S100A8 | S100A9 | SAA1 | SAA2 | SCGB3A2 | SCRT2 |
| SEMA7A | SERPINA4 | SERPINA7 | SERPINB13 | SERPINB3 | SERPINB4 | SERPINB5 | SERPINB7 |
| SFN | SFTPA1 | SFTPA2 | SH2D1A | SHCBP1 | SIGLEC14 | SKA1 | SLC13A2 |
| SLC17A8 | SLC1A6 | SLC25A48 | SLC35D3 | SLCO1A2 | SLITRK1 | SLN | SMCO1 |
| SMIM31 | SMPX | SMYD1 | SOST | SOX11 | SP8 | SPACA3 | SPATA31D1 |
| SPC24 | SPO11 | SPOCD1 | SPRR2A | SPRR2D | SRD5A2 | STEAP1 | STRA8 |
| STRIT1 | SULT1E1 | SVOP | SYT8 | TAAR2 | TAC3 | TAF11L11 | TAFA4 |
| TARM1 | TAS1R2 | TBX20 | TCN1 | TEX13A | TEX33 | TFAP2A | TFDP3 |
| TFPI2 | TGM5 | TMEM158 | TMEM207 | TMEM40 | TNFAIP6 | TNIP3 | TNNI2 |
| TNNT1 | TNR | TOP2A | TP63 | TPX2 | TRIM46 | TRIML1 | TRIML2 |
| TRIP13 | TROAP | TTK | TTPA | TUBA3C | TWIST1 | TXNDC8 | UBE2C |
| UCN2 | UGT2B15 | UGT3A1 | UHRF1 | UNC45B | URAD | VCAN | VRTN |
| WFDC9 | WNT2 | WNT7B | WT1 | XAGE5 | XDH | XIRP2 | ZIC1 |
| ZIC2 | ZIC4 | ZNF365 | ZP4 | ZPBP2 | | | |

Eight genes that are upregulated in both RAS-like and aggressive non-metastatic samples, and 549 genes that are upregulated in both BRAF-like and aggressive non-metastatic samples.

### Example 3-MAP Score Identifies CAF-Rich Microenvironments

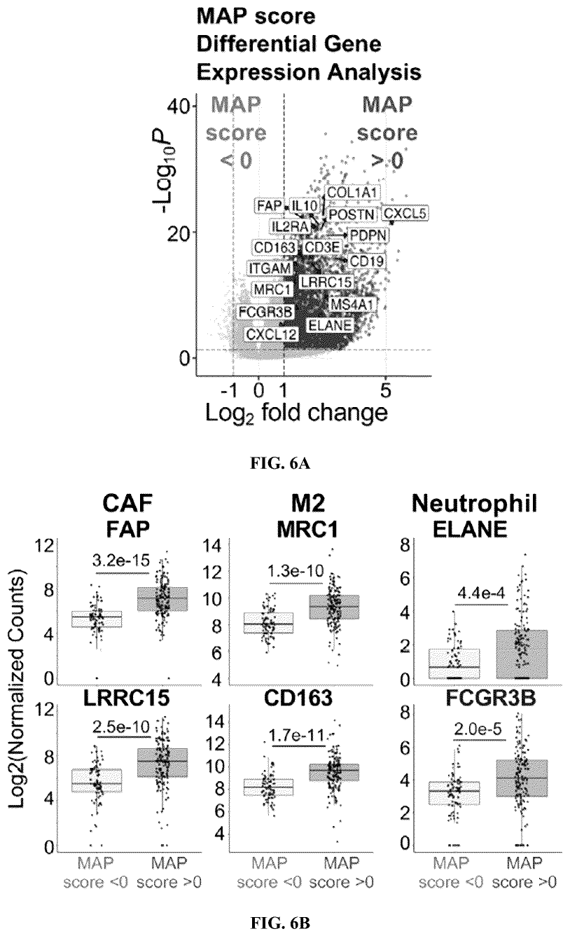

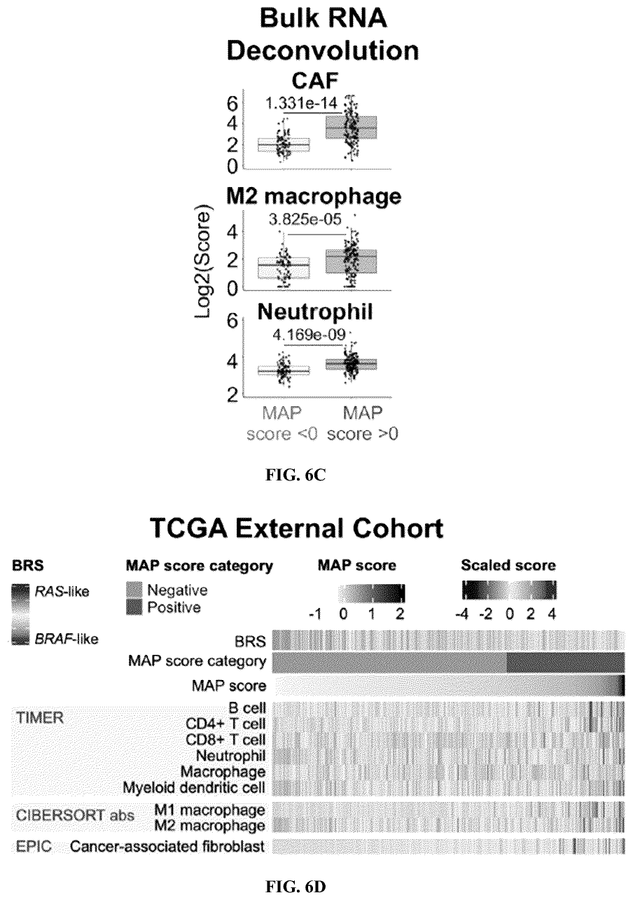

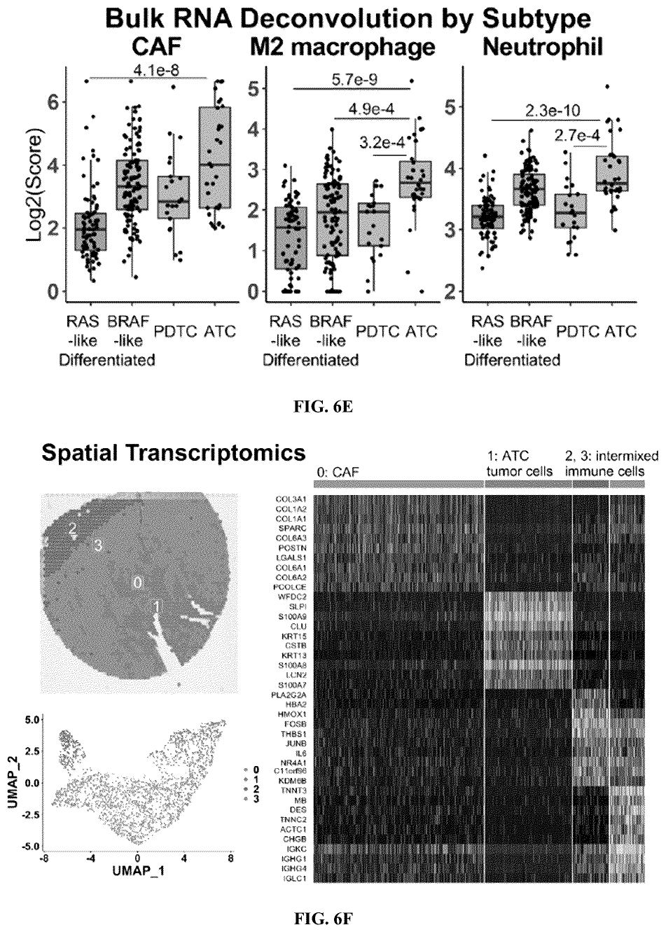

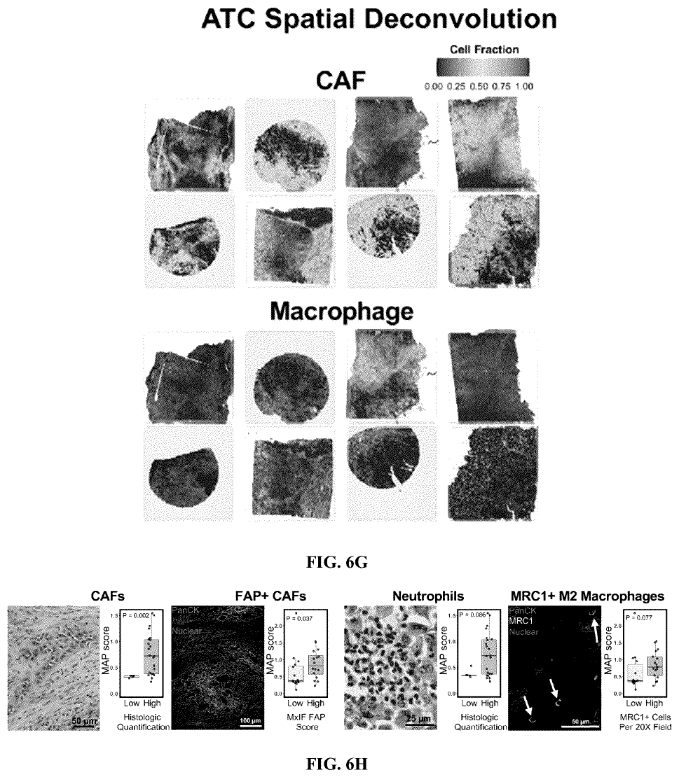

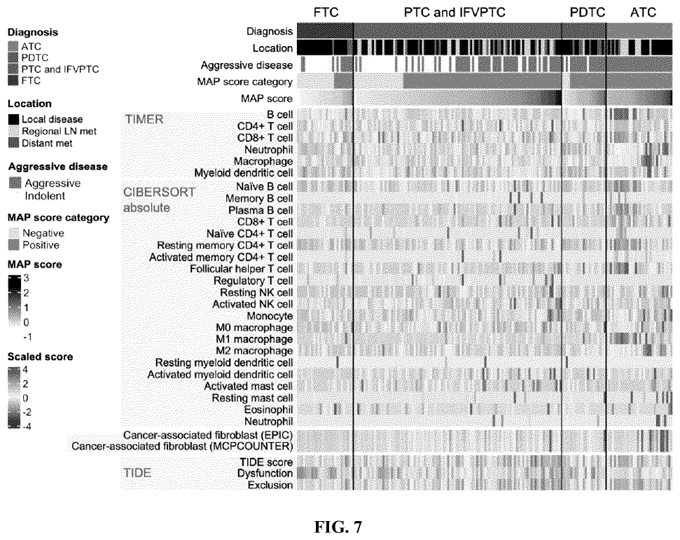

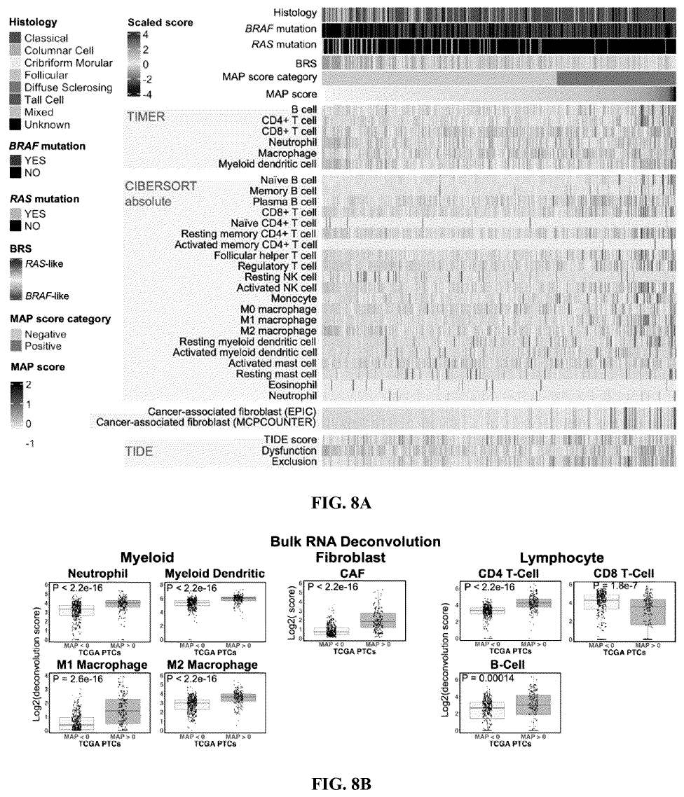

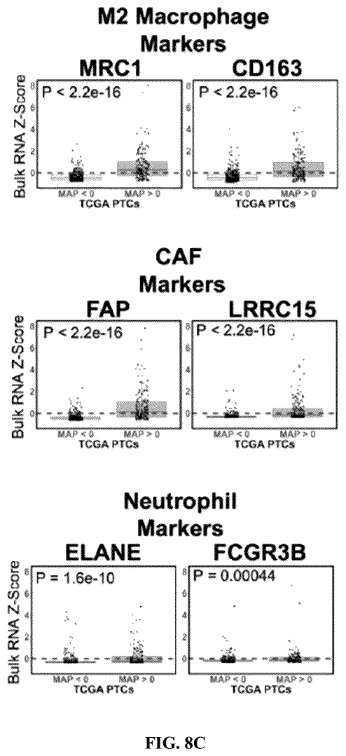

Based on the presence of extracellular matrix and immune-related genes in our MAP score, as well as the reported functional roles of CAFs in disease progression, 45 we further explored the stromal infiltrate of tumors with positive MAP score. Differential gene expression comparing positive and negative MAP score samples in our cohort showed enrichment of inflammatory genes in MAP-positive samples (FIG. 6A), including markers of specific immune cell infiltrates such as CAFs, M2 macrophages, and neutrophils (FIG. 6B). To confirm the stromal infiltrate in our cohort, we predicted infiltrating cell populations from bulk RNA sequencing using the immune deconvolution tools TIMER, CIBERSORT, EPIC, and MCPCOUNTER. Again, we observed strong enrichment of CAFs, M2 macrophages, and neutrophils in MAP-positive tumors (FIG. 6A and FIG. 7). To further validate the association of the MAP score with this unique stromal infiltrate, we utilized a large external cohort of well-differentiated PTC samples in the TCGA (FIG. 6D and FIG. 8A-8C). We found enrichment of CAFs, neutrophils, and M2-macrophages in MAP-positive PTC tumors, with enrichment of CD8+ T cells in MAP-negative tumors (FIG. 8A-8C).

As MAP scores are highest in ATCs, we next assessed whether ATCs had higher CAF infiltrates compared to other thyroid cancers. Comparison of these infiltrates across major thyroid cancer subtypes revealed that ATCs had the highest predicted CAF, M2 macrophage, and neutrophil infiltrates. RAS-like well-differentiated cancers and PDTCs had the lowest predicted levels of these stromal cells (FIG. 6E). To spatially confirm this infiltrate identified in ATCs from bulk sequencing data, we next performed spatial transcriptomics on eight ATC samples from our cohort. Clustering showed robust, distinct populations of tumor cells and CAFs in all ATC samples (FIG. 6F) but did not clearly detect separate populations of other immune cell subsets. As spatial transcriptomics is not single-cell resolution, clustering was unable to detect smaller populations of intermixed immune cell subsets. To overcome this limitation, we used a spatial deconvolution algorithm, SpaCET, to deconvolute the immune cell populations present within individual spatial capture areas. Exploration of immune cell populations with SpaCET confirmed robust CAF and macrophage infiltrate in all eight ATC specimens (FIG. 6G). As the gold-standard method of confirmation of neutrophil, CAF, and M2 macrophage infiltrates, we performed blinded pathologist review and scoring of H&E and immunofluorescence staining for fibroblast activation protein (FAP) (CAF marker) and MRC1 (M2 macrophage marker) in all ATCs of our cohort (FIG. 6H). While all ATCs had positive MAP scores, samples with high levels of CAFs, neutrophils, and M2 macrophages had higher MAP positivity. Overall, these findings suggest a strong association between MAP score and CAF, M2 macrophage, and neutrophil infiltrates across thyroid cancer subtypes.

### Example 4—MAP Score Highlights ATCs that May Respond to Immunotherapy

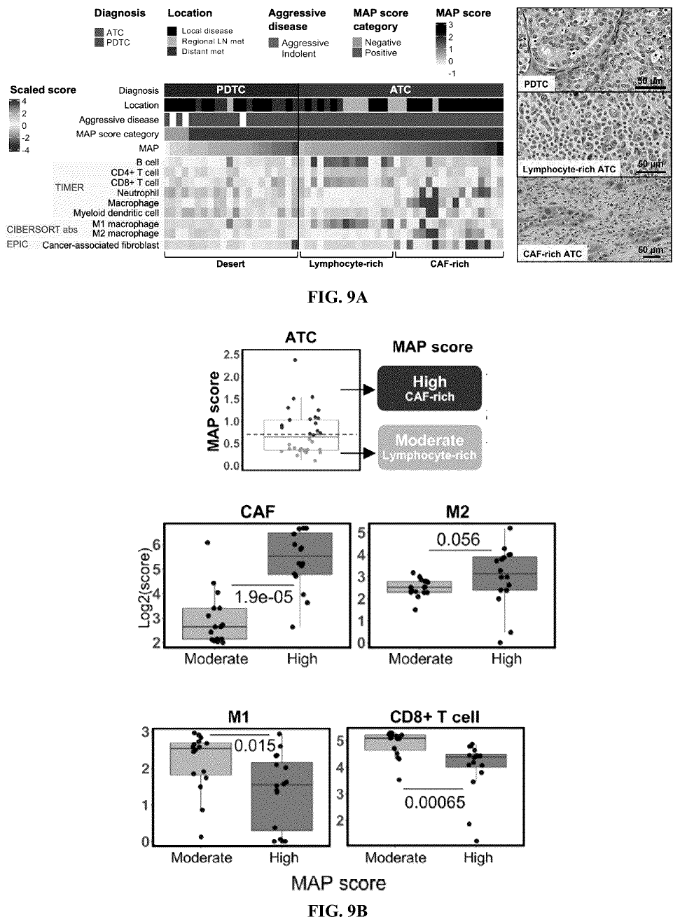

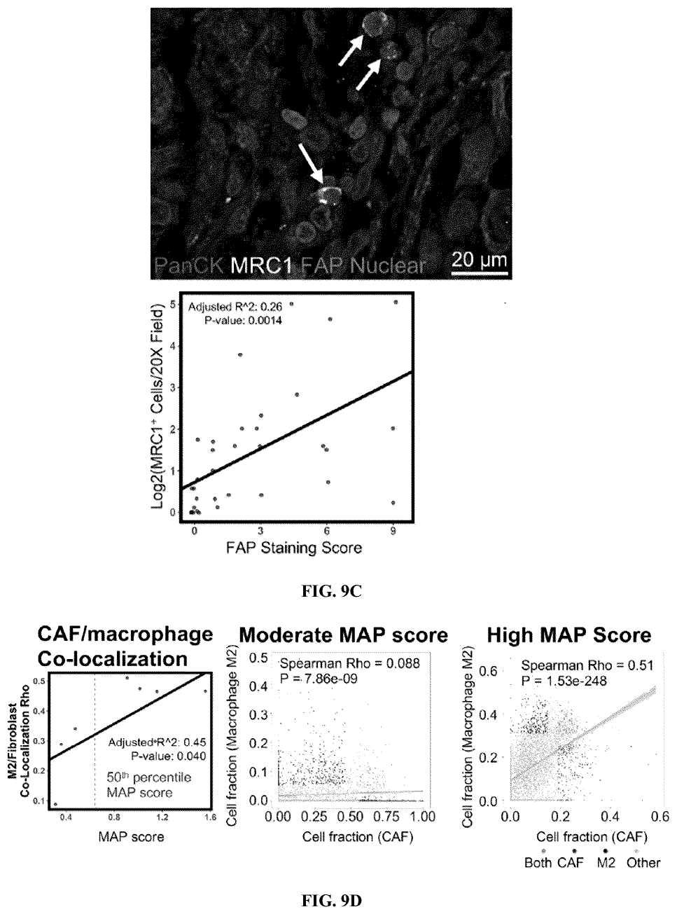

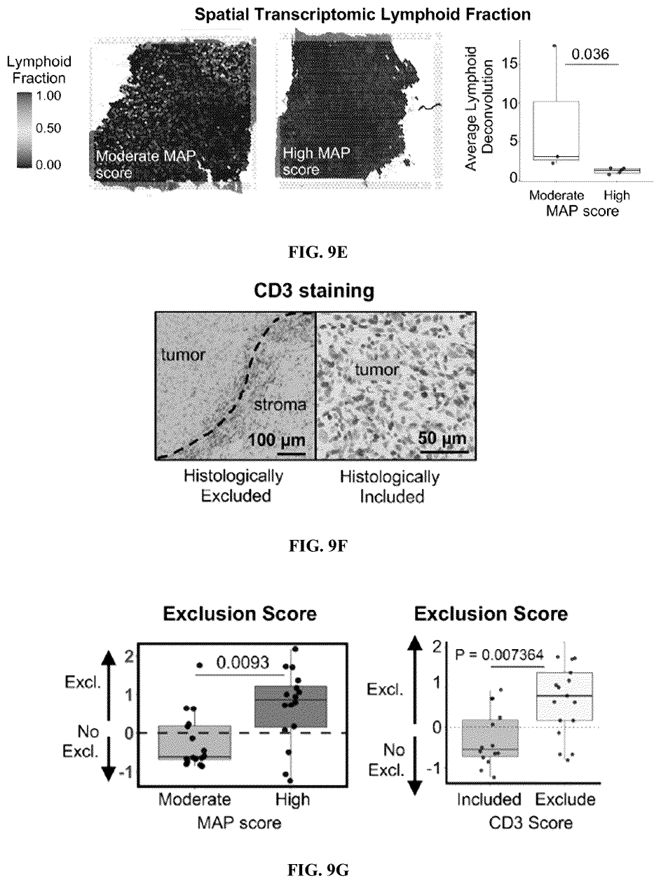

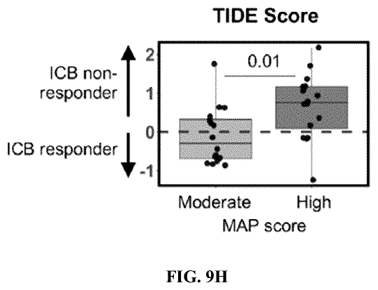

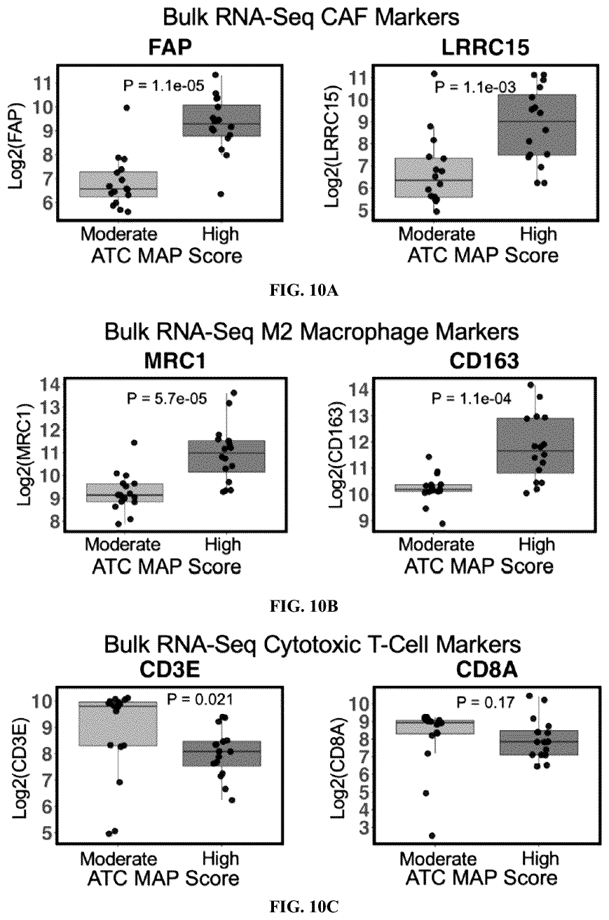

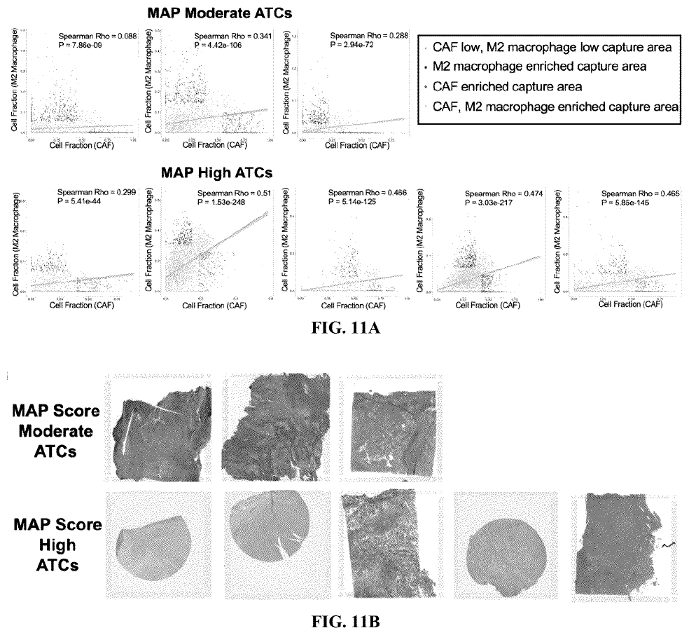

Given the recent clinical trials of immunotherapy for transformed tumors (PDTCs and ATCs), we further investigated immune cell subsets within these thyroid cancer subtypes. Immune deconvolution of bulk RNA sequencing from transformed tumors revealed three striking patterns of immune cell infiltrate: immune dessert, lymphocyte rich, and CAF rich (FIG. 9A). Regardless of MAP positivity, PDTCs showed low scores for immune infiltration, suggesting “immune desert” microenvironments. This finding is similar to prior reports of immune-cold PDTCs. 21,46 In contrast, ATCs were rich in stomal and immune cells, displaying either a lymphocyte/M1 macrophage-rich or CAF/M2 macrophage-rich infiltrate. While both metastatic and thyroid-localized ATCs demonstrated lymphocyte-rich microenvironments, CAF-rich microenvironments were more commonly seen in ATC samples from the thyroid and surrounding soft tissues. The lymphocyte-rich stroma in ATC strongly correlated with moderate MAP score. Using a 50th percentile MAP score cutoff, we categorized ATCs as either having a moderate MAP score or a high MAP score and found a significant association with lymphocyte-rich versus CAF-rich microenvironments, respectively (FIG. 9B and FIG. 10A-10C).

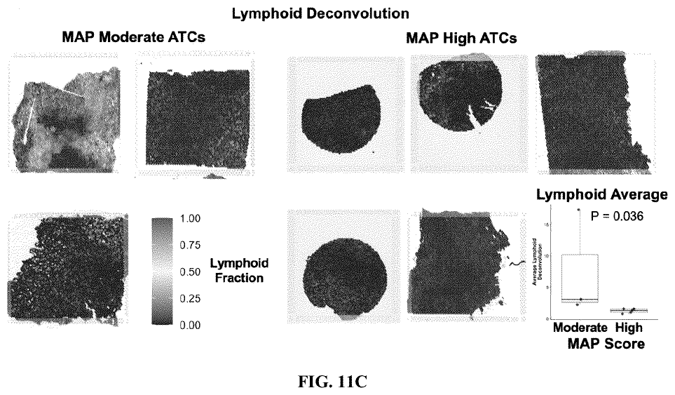

To further explore the spatial association of CAFs and M2 macrophages in ATC, we performed multiplex immunofluorescence for the CAF marker FAP and the M2 macrophage marker MRC1 across all ATCs in our cohort. We found a strong correlation between the abundance of MRC1-positive (MRC1+) macrophages and FAP-positive (FAP+) CAFs in ATCs. While the FAP+ CAFs abutted tumor cells, the MRC1+ macrophages were predominantly localized within the tumor stroma adjacent to fibroblasts (FIG. 9C). To quantitatively analyze co-localization of CAFs and M2 macrophages, we evaluated the correlation between predicted CAF and M2 macrophage frequency for individual capture areas within our spatial transcriptomics data. ATC samples had significant spatial correlation between CAFs and M2 macrophages. However, the magnitude of the correlation was more pronounced in tumors with higher MAP scores, suggesting greater CAF/M2 macrophage co-localization within high MAP tumors (FIG. 9D and FIG. 11A).

We next verified the abundance of infiltrating lymphocytes in moderate-MAP ATCs using spatial transcriptomics and immunohistochemical staining. Using our ATC spatial transcriptomic samples, we identified increased abundance of lymphoid populations in samples with moderate versus high MAP scores (FIG. 9E and FIG. 11B-11C). We also performed immunohistochemical staining and blinded scoring of CD3 stains from whole tumor sections of all ATCs in our cohort. We found two patterns of T cell infiltrate with CD3 staining: CD3 inclusion and CD3 exclusion (FIG. 9F). This histologic assessment strongly correlated with the T cell exclusion prediction algorithm, tumor immune dysfunction exclusion (TIDE), performed on the same ATC samples. TIDE is an immune deconvolution tool that uses bulk RNA sequencing data to provide estimates of T cell exclusion, dysfunction, and predicted response to immune checkpoint blockade (ICB) therapy. 47 Our TIDE exclusion results and CD3 staining strongly supported the association of moderate MAP score with T cell inclusion and high MAP score with T cell exclusion (FIG. 9F-9G). Finally, we utilized TIDE to predict potential response to ICB immunotherapy. As anticipated, ATCs with moderate MAP score and T cell inclusion were predicted to respond to immunotherapy, whereas ATCs with high MAP score and T cell exclusion were predicted to be non-responders (FIG. 9H). The data suggest that MAP scoring predicts ATC stromal infiltrate and potential response to ICB therapy.

### Example 5—MAP Score for Thyroid Cancer Outcome Prediction

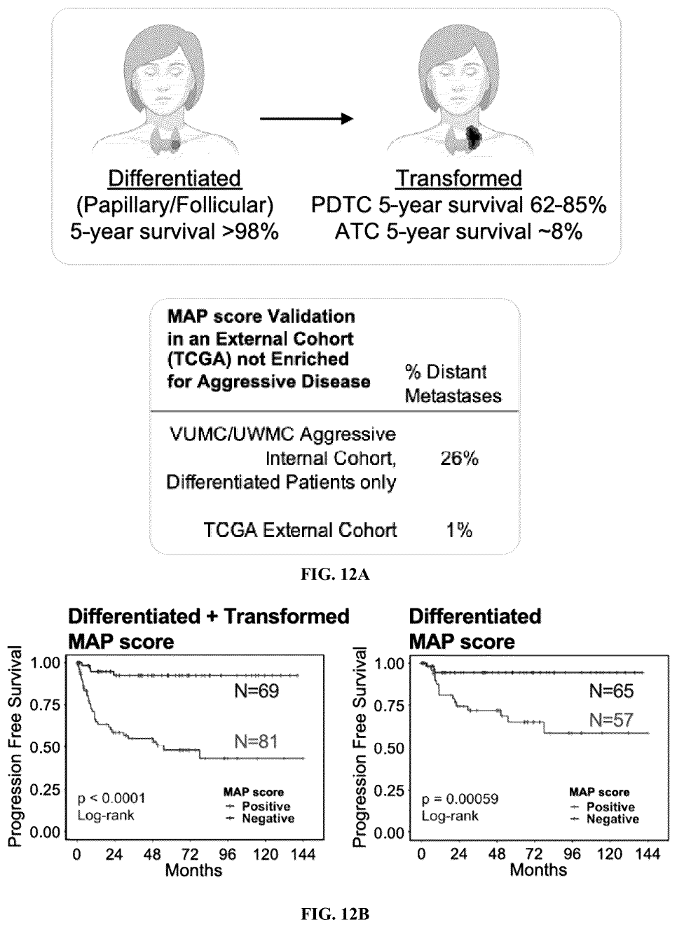

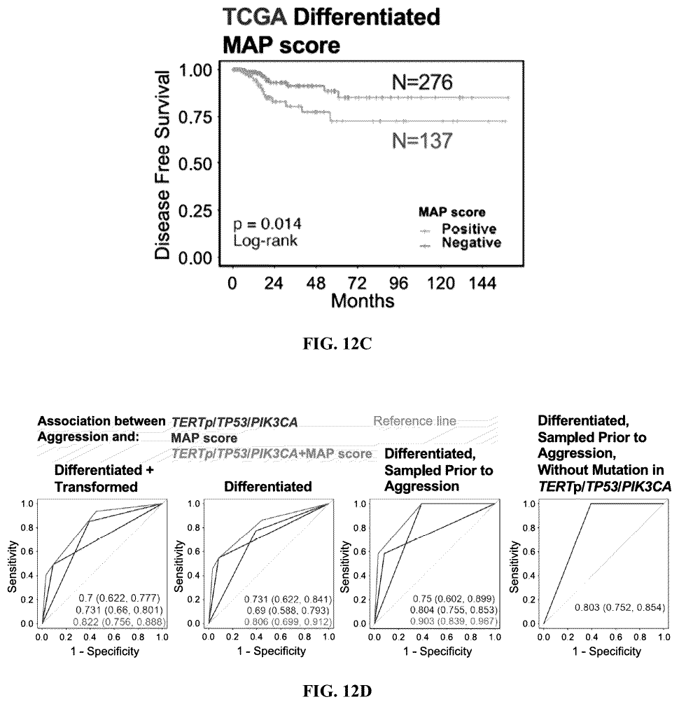

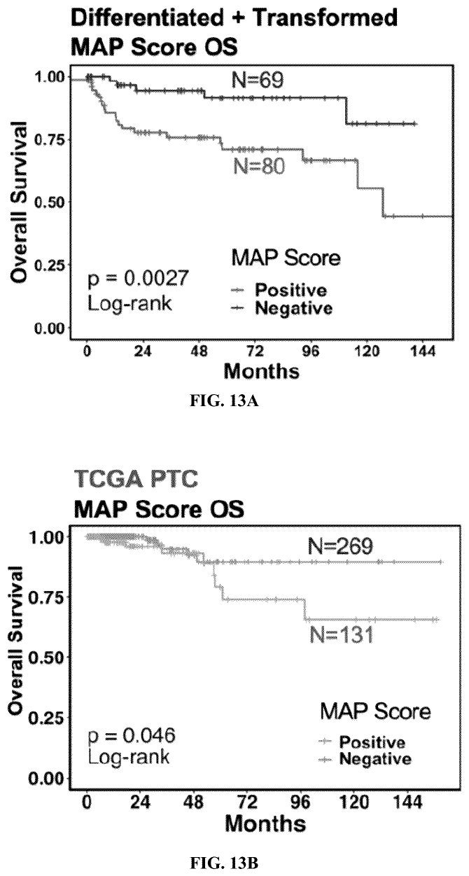

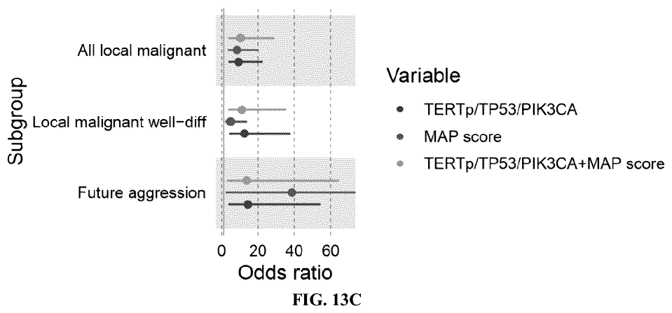

Thus far, we have calculated a MAP score that is enriched for microenvironment genes. We next assessed whether MAP score could be used as a robust predictor of aggressive disease. To perform outcome prediction, we used two cohorts: our cohort with enrichment for transformed and metastatic disease and the TCGA cohort, which is primarily composed of patients with non-metastatic (less aggressive) disease. The benefits of utilizing two cohorts were 2-fold, as it allowed (1) the ability to include an external cohort with the TCGA tumors and (2) the ability to include our cohort enriched in well-differentiated tumors with aggressive behavior such as distant metastases (26% of our patients vs. 1% of TCGA patients, FIG. 12A). Survival analysis using our cohort with both well-differentiated and transformed tumors, as well as with well-differentiated tumors alone, showed that patients with positive MAP scores had significantly worse survival (FIG. 12B and FIG. 13A). Despite having primarily patients with local disease, the TCGA cohort also showed a significant decrease in disease-free and overall survival in tumors with positive MAP score (FIG. 12C and FIG. 13B).

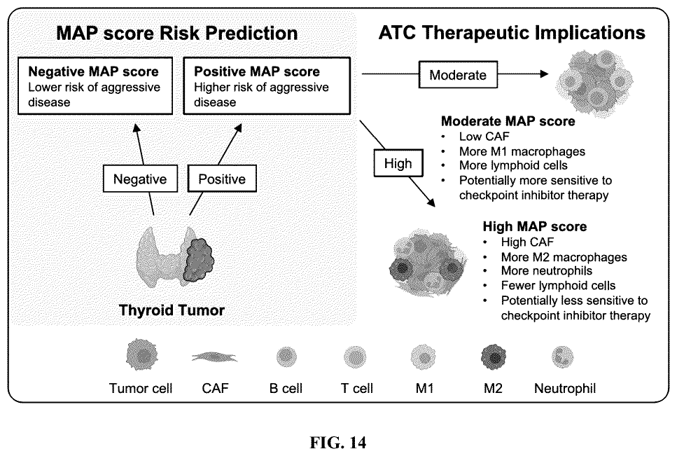

To further assess the ability of the MAP score to predict disease progression, we performed generalized linear models with penalized maximum likelihood estimation on our cohort. Score performance was assessed on three groups of local disease malignant samples: all malignant samples, well-differentiated malignancies, and well-differentiated malignancies that were resected prior to any evidence of disease progression. Inclusion of malignancies resected prior to disease progression allowed for prediction of future disease risk. We compared these scores to the predictive capacity of three common high-risk mutations: TP53, TERTp, and/or PIK3CA. Both the MAP score and the mutation scores performed similarly and were found to be good predictors of aggression (FIG. 12D and FIG. 13C and Table 9). Significantly, combining the MAP score with high-risk mutations provided the greatest predictive power by area under the receiver operating characteristic curve (FIG. 12D, Table 9). In addition, the MAP score provided aggressive disease prediction in samples lacking these known high-risk mutations (FIG. 12D, Table 9). Altogether, we show that the molecular prediction of disease outcome is improved with the inclusion of both high-risk mutations and gene signatures that incorporate stromal markers of aggressive disease. Molecular prediction of the stromal infiltrate could be highly useful in enhancing outcome prediction in thyroid cancer (FIG. 14).

TABLE 9
Generalized linear model aggression prediction results.

| Dataset | Variable | p | OR (CI) | | AUC (CI) | |
|---|---|---|---|---|---|---|
| Differentiated + | TERTp/TP53/PIK3CA | 0.000 | 9.239 | (3.807, 22.421) | 0.7 | (0.622, 0.777) |
| Transformed | mutation | | | | | |
| | MAP score | 0.000 | 8.407 | (3.481, 20.307) | 0.731 | (0.66, 0.801) |
| | TERTp/TP53/PIK3CA | 0.000 | 10.345 | (3.685, 29.038) | 0.822 | (0.756, 0.888) |
| | mutation + MAP score | | | | | |
| Differentiated | TERTp/TP53/PIK3CA | 0.000 | 12.535 | (4.172, 37.659) | 0.731 | (0.622, 0.841) |
| | mutation | | | | | |
| | MAP score | 0.003 | 4.917 | (1.723, 14.036) | 0.69 | (0.588, 0.793) |
| | TERTp/TP53/PIK3CA | 0.000 | 11.174 | (3.518, 35.491) | 0.806 | (0.699, 0.912) |
| | mutation + MAP score | | | | | |
| Differentiated, | TERTp/TP53/PIK3CA | 0.000 | 14.358 | (3.785, 54.468) | 0.75 | (0.602, 0.899) |
| Sampled Prior to | mutation | | | | | |
| Aggression | MAP score | 0.013 | 38.637 | (2.169, 688.234) | 0.804 | (0.755, 0.853) |
| | TERTp/TP53/PIK3CA | 0.001 | 13.715 | (2.917, 64.478) | 0.903 | (0.839, 0.967) |
| | mutation + MAP score | | | | | |
| Differentiated, | MAP score | 0.061 | 16.888 | (0.881, 323.815) | 0.803 | (0.752, 0.854) |
| Sampled Prior to | | | | | | |
| Aggression, Without | | | | | | |
| Mutation in | | | | | | |
| TERTp/TP53/PIK3CA | | | | | | |

Generalized linear model aggression prediction, subset by patients possessing differentiated and transformed lesions, differentiated lesions, differentiated lesions collected prior to aggression, and differentiated lesions collected prior to aggression without mutation in TERTp/TP53/PIK3CA. NIFTP lesions were included with malignant lesions. Local disease location samples only. Abbreviations: OR = Odd ratios; CI = 95% confidence interval; AUC = Area Under the ROC Curve.

### Example 6—Discussion Regarding Examples 1-5

The primary focus of thyroid nodule molecular profiling has been on malignancy prediction, rather than prediction of disease progression, recurrence, or therapy response. Recent studies have made significant progress in predicting patients with higher-risk disease by focusing on high-risk alterations such as mutations in TERTp, TP53, and PIK3CA, often in combination with BRAF V600E. 27 However, many patients with metastatic, recurrent, or progressive thyroid cancer lack such known high-risk mutational biomarkers. To better understand the pathogenesis of thyroid cancer progression and to identify additional biomarkers, we sequenced a diverse collection of thyroid lesions from a large patient cohort enriched for aggressive disease. As expected, TERTp, TP53, and PIK3CA mutations were associated with decreased PFS. However, our findings suggest that approximately 40% of patients with aggressive well-differentiated tumors lack these previously identified high-risk mutations. Based on these findings, we generated the MAP score, which was associated with outcome in both our cohort and the TCGA PTC cohort. We found that, when utilized in combination with known high-risk mutations, MAP score improves the prediction of thyroid cancer aggressiveness. Importantly, the MAP score could also potentially provide outcome prediction in patients lacking these mutations.

In addition to outcome prediction, the MAP score also offered an assessment of immune infiltrate, which could be useful in identifying ATC patients who might respond to ICB immunotherapy. Clinical trials have recently showed some moderate response of ATC to ICB therapy. 33,34,35,36,37,38 Immune profiling with a molecular signature such as the MAP score could help identify ICB-responsive ATC patients. MAP-predicted differential immune infiltration also highlights important biology that may shed light on the drivers of aggressive disease. CAFs, macrophages, and neutrophils have been implicated as key regulators of anti-tumor immunity and have substantial crosstalk with tumor cells, indicating their ability to influence tumor growth and response to therapy. 48 As such, the robust infiltration of CAFs, neutrophils, and M2 macrophages in MAP-high tumors may play a key role in thyroid cancer progression. Additional research is needed to explore whether differing CAF populations and/or immune infiltrate compositions could be used to inform the development of new targeted therapies for ATC patients.

In conclusion, our findings identify the stromal microenvironment as an important component of outcome in thyroid cancer. We show that incorporation of a molecular signature including stromal genes with standard mutational analysis could improve risk-stratification and may even predict immunotherapy response in ATC patients. In the future, we envision a testing platform utilizing both mutational and stromal microenvironment data for outcome and ICB response prediction. Continuing research on the stromal microenvironment of thyroid cancer has the potential to improve care of thyroid cancer patients, identify novel therapeutic targets for aggressive disease, and potentially help prevent ATC, one of the most aggressive forms of thyroid cancer. Similar molecular tests could potentially provide predictive value for other stromal-rich cancers. While more research is needed, assessment of stromal microenvironment genes has the potential to deepen our understanding of cancer biology, redefine tumor classification, and estimate poor outcome risk for patients across a wide range of solid tumors.

### Example 7—Experimental Model and Study Participant Details

Following Institutional Review Board approval from Vanderbilt University Medical Center (VUMC) and the University of Washington Medical Center (UWMC), all consecutive cases of advanced thyroid cancer (including well-differentiated tumors with distant metastases, ATCs, and PDTCs) resected at VUMC between Oct. 14, 2005 and Jan. 14, 2020, and well-differentiated malignancies with distant metastases at UWMC between Oct. 11, 2002 and Jul. 14, 2017 were included in the study. We define well-differentiated thyroid tumors as including Follicular Thyroid Carcinoma, FTC; Oncocytic Thyroid Carcinoma, OTC, Noninvasive Follicular Thyroid Neoplasm with Papillary-like Nuclear Features, NIFTP, Encapsulated Follicular Variant Papillary Thyroid Carcinoma, EFVPTC, Papillary Thyroid Carcinoma, PTC, and Infiltrative Follicular Variant Papillary Thyroid Carcinoma, IFVPTC. All such cases of aggressive thyroid cancers with sufficient FFPE tissues and tumor percentage were included in this study (N=123 samples). Samples (collected during the same data range) from age-matched patients with well-differentiated malignant thyroid lesions without distant metastases (N=112 samples), multinodular goiters (N=21 samples), patients with clinically diagnosed Hashimoto thyroiditis (N=14 samples), and benign neoplasms (N=42 samples) were also included for comparison. In all, 312 samples from 251 different patients were included in this study (VUMC, N=292 samples and UWMN, N=20 samples). Among the patients there are 163 females. For analyses, samples were binned into non-neoplastic (MNG, HT), neoplastic (FA, OA), well-differentiated malignancies (FTC, OTC, EFVPTC, IFVPTC, PTC), and transformed malignancies (PDTC, ATC). As NITFP is not yet clearly defined as either benign or malignant, it was grouped with our well-differentiated malignancies. Diagnostic criteria are based on WHO and ATA guidelines. Each specimen's histopathology was reviewed by three board-certified pathologists (VW, MM, KE).

### Example 8—Clinical Data

Manual chart review was performed (GX, ML, JG, EH) to gather additional/pertinent patient demographics (e.g., race), clinical histories (e.g., prior exposures to ionizing radiation), treatment courses (e.g., types of surgeries), tumor details (e.g., size), and outcomes (e.g., survival).

### Example 9—Patient Outcomes and Survival Analyses

Responses to therapy (typically surgery and radioactive iodine) and patient outcomes were categorized in accordance with the latest American Thyroid Association guidelines3 and are detailed below. Complex and equivocal cases were discussed (GX, ML, JG, VW) until unanimous consensus was achieved. For aggressive disease classification, patients were grouped into the two categories described below.

### Example 10—Indolent

Includes patients with no evidence of disease (NED), indeterminate disease, persistent disease, or recurrent disease in remission. NED was defined by undetectable thyroglobulin (Tg) level, lack of circulating anti-thyroglobulin antibodies (aThyG), and a thyroid ultrasound indicating no evidence of disease. Patients without imaging follow-up were determined to be NED by laboratory testing (undetectable Tg or aThyG) alone. Imaging alone (without labs) was only sufficient for NED if the patient had a hemithyroidectomy or no radioactive iodine. Indeterminate disease was defined by with stable/detectable Tg<1.0 ng/ml, stimulated Tg<10 ng/mL, positive aThyG levels that were not increasing, imaging without Tg labs, and/or inconclusive imaging. Persistent disease includes stable Tg>1.0 ng/mL, stimulated Tg>10 ng/mL, and/or a persistent lesion by imaging that did not increase in size over multiple years of follow-up. Recurrent disease in remission includes malignancies that could be measured (via imaging or laboratory testing) after a designation of NED but were treated with local intervention and followed by stable or decreasing tumor size or Tg.

### Example 11—Aggressive Disease

Defined by local disease recurrence without stabilization of disease or remission following subsequent localized treatment; increasing lesion size after initial therapy; biopsy demonstrating transformation to ATC; or distant metastasis after initial therapy completion. Patients with transformed disease or metastatic disease at presentation were categorized as having aggressive disease.

### Example 12—Survival Analyses

For progression-free survival (PFS) analyses, the interval between the completion of initial therapy to the date of progression was calculated. The date of disease progression was determined by the first date of either an increasing Tg (in an appropriately thyroid-stimulating hormone suppressed patient) or the increase in size of a lesion by imaging. All patients that were determined to be progressive by Tg had subsequent imaging evidence of progressive disease. For patients without progression, the date of last follow-up was used to determine PFS—and the data appropriately censored. Patients with no follow-up after therapy were omitted from analysis. Overall survival similarly was calculated from the completion of initial therapy to the date of death or date of last follow-up (also censored in the case of a living patient). For well-differentiated tumors, the date of initial therapy completion was either 1) the date of post-operative radioactive iodine administration or 2) the date of surgery for low-risk tumors that did not require post-operative radioactive iodine (e.g., NIFTP). For undifferentiated tumors (PDTC and ATC), the date of surgery was used as the initial therapy completion date.

### Example 13—DNA Sequencing and Mutational Analysis

Nucleic acids were extracted using the COVARIS truXTRAC FFPE Total NA Kit per the manufacturer's instructions (COVARIS, Woburn, MA). DNA libraries were built using the NEB Ultra II DNA Library Prep Kit per the manufacturer's instructions (NEB, Ipswich, MA). Sequencing was performed at the Vanderbilt Technologies for Advanced Genomics (VANTAGE) core facility on an Illumina NovaSeq 6000 platform using the IDT xGen Exome Research Panel (Illumina, San Diego, CA). Raw 150 bp paired-end reads were trimmed to remove adapter sequences using Cutadapt (v2.10) 57 and the quality of the reads before and after trimming was checked by

FastQC. (www.bioinformatics.babraham.ac.uk/projects/fastqc).⁵⁸ Trimmed reads were aligned to hg38 genome using Burrows-Wheeler Aligner (v0.7.17-r1188).⁵⁹ GATK v. 4.1.8.1 was used to remove duplicate reads, perform base quality score recalibration and variants discovery.⁶⁰ Variant calling was first performed on individual samples using HaplotypeCaller in gVCF mode, all samples were jointly genotyped, and variant filtering was performed with VQSR. Variant annotation was conducted with ANNOVAR (v2018-04-16). 77 Variants with minor allele frequency≥0.1% in at least one of the ExAC (Exome Aggregation Consortium), 49 1000G (1000 Genomes Project), 49 and gnomAD (Genome Aggregation Database)50 databases were filtered out. BRAF, RAS, TP53, and PIK3CA mutations were evaluated according to the standards and guidelines for the reporting of sequence variants in cancer by the Association for Molecular Pathology, American Society of Clinical Oncology, and the College of American Pathologists. 78 Average depth and coverage of whole exome sequencing was 157× and 91×, respectively.

TERT promoter alterations C228T and C250T were probed using Sanger sequencing with primers: forward+T7 tail and reverse+M13F tail.⁵⁶ and the HotStarTaq DNA Polymerase kit (QIAGEN, Hilden, Germany). Thermal cycling conditions were as follows: 95° C. (15 min), followed by 35 cycles of 94° C. (30 s), 56° C. (30 s), and 72° C. (20 s), followed by 72° C. (10 min) and 4° C. hold. Purified PCR products were analyzed using Sanger sequencing (GENEWIZ, South Plainfield, NJ).

### Example 14—RNA Sequencing and Tumor-Infiltrating Immune Cell Deconvolution

Nucleic acids were extracted using the COVARIS truXTRAC FFPE Total NA Kit as above (COVARIS, Woburn, MA). Illumina TruSeq mRNA sequencing libraries were prepared and sequenced at VANTAGE on a NovaSeq 6000 platform Raw (Illumina, San Diego, CA). Raw 150 bp paired-end reads were trimmed to remove adapter sequences using Cutadapt (v2.10)57 and aligned to the GENCODE GRCh38. p13 genome51 using STAR (v2.7.8a). 61 GENCODE v38 gene annotations were provided to STAR to improve the accuracy of mapping. Quality control on both raw reads and adaptor-trimmed reads was performed using FastQC. 58 featureCounts (v2.0.2) 62 was used to count the number of mapped reads to each gene. Significantly differential expressed genes with FDR-adjusted p value<0.05 and absolute fold change>2.0 were detected by DESeq2 (v1.30.1)63 and visualized with R package EnhancedVolcano (1.18). 64 The R package Heatmap365 was used for cluster analysis and visualization. Gene Ontology was performed on differentially expressed genes using the Gene Ontology Consortium resource. 52,53 Gene set enrichment analysis was performed using GSEA (v4.1.0) 66 on the msigdb v7.1 database. TIMER2.0 (timer.cistrome.org/), a web-based deconvolution program capable of estimating tumor-infiltrating immune cells based on gene expression profiles across diverse cancer types67 was used. TIMER 2.0 was run using THCA (Thyroid Carcinoma) as the cancer type gene signature. TIMER 2.0 immune deconvolution scores used include those from CIBERSORT-Abs, 79 EPIC, 79 and MCPCOUNTER. 80 Descriptive results were plotted using the R package ggplot2.68 In addition, we used the computation tool TIDE (tide.dfci.harvard.edu/)⁴⁷ to estimate immune checkpoint blockade response based on gene expression data. The TIDE response prediction module was run using the following settings: Cancer type=Other, Previous Immunotherapy=No. TIMER and TIDE score heatmaps were generated using R package ComplexHeatmap. 69

### Example 15—Fusion Analysis

The STAR-Fusion (v2.7.8a) pipeline⁷⁰ was used to align and map paired-end RNA-seq reads to the human genome (GRCh38_gencode_v37) using parameters optimized to capture fusion transcripts.⁸¹ FusionInspector, a component of the STAR-Fusion suite was used to validate fusion transcripts in silico. Manual review of RNA data was performed using the Integrated Genomics Viewer⁷¹ and two additional RET fusions were identified by blasting soft clip reads to the human genome.

The following parameters were used to run STAR-Fusion:
STAR—genomeDir—outReadsUnmapped None—chimSegmentMin 12—chimJunctionOverhangMin 8—chimOutJunctionFormat 1—alignSJDBoverhangMin 10-alignMatesGapMax 100000-alignIntronMax 100000—alignSJstitchMismatchNmax 5-1 5 5.
   runThreadN 8—outSAMstrandField intronMotif—outSAMunmapped Within—alignInsertionFlush Right—alignSplicedMateMapLminOverLmate 0—alignSplicedMateMapLmin 30—outSAMtype BAM Unsorted—outSAMattrRGline ID: GRPundef—chimMultimapScoreRange 3—chimScoreJunctionNonGTAG—4—chimMultimapNmax 20—chimNonchimScoreDropMin 10.
   peOverlapNbasesMin 12—peOverlapMMp 0.1—genomeLoad NoSharedMemory—twopassMode Basic.
Command.
STAR—genomeDir.
   outReadsUnmapped None.
   chimSegmentMin 12.
   chimJunctionOverhangMin 8.
   chimOutJunctionFormat 1.
   alignSJDBoverhangMin 10.
   alignMatesGapMax 100000.
   alignIntronMax 100000.
   alignSJstitchMismatchNmax 5-1 5 5.
   runThreadN 8.
   outSAMstrandField intronMotif
   outSAMunmapped Within.
   alignInsertionFlush Right.
   alignSplicedMateMapLminOverLmate 0.
   alignSplicedMateMapLmin 30.
   outSAMtype BAM Unsorted.
   outSAMattrRGline ID: GRPundef.
   chimMultimapScoreRange 3.
   chimScoreJunctionNonGTAG—4.
   chimMultimapNmax 20.
   chimNonchimScoreDropMin 10.
   peOverlapNbasesMin 12.
   peOverlapMMp 0.1.
   genomeLoad NoSharedMemory
   twopassMode Basic.

### Example 16—Calculation of RNA Scores

BRAF-RAS score calculation. The BRAF-RAS score (BRS) was calculated from a previously defined list of 71 genes 14 using bulk RNA-sequencing data transformed into Z score format. 69 of the 71 genes in the originally published score were covered in the sequencing data and were used here. In brief, BRAF-mutant and RAS-mutant centroids were calculated and used to generate a BRS for each sample.

Calculating BRAF-mutant and RAS-mutant centroids. BRAF-mutant ([B]) and RAS-mutant ([R]) centroids were calculated from PTCs and FVPTCs with BRAF V600E and RAS mutations (NRAS, HRAS, or KRAS). The centroids consisted of vectors of the median expression of each of the 69 BRS genes for each group (BRAF or RAS mutant).

Calculating BRS for each sample. For each sample, a vector containing the expression of the 69 BRS genes was generated ([S]). The normalized squared Euclidean distance between [S] and [B] and [S] and [R] was calculated. Finally, the BRS was calculated as the difference between these normalized squared Euclidean distances such that a negative value indicated a BRAF-like sample and positive value a RAS-like sample, as shown below. Note that | [S]-[B] | and [S]-[R] | indicate normalized squared Euclidean distances.

$$BRS ( S ) = \vert [ S ] - [ B ] \vert - \vert [ S ] - [ R ] \vert$$

Thyroid differentiation score calculation. The thyroid differentiation score, or TDS, was calculated from the mRNA expression levels of 16 genes related to thyroid function and metabolism, as previously described. 14 To calculate TDS, the variance stabilized expression data were subtracted by the median across all tumor samples. Next, the TDS was calculated from the average values across the 16 genes in each tumor.

ERK activity score calculation. The ERK score was calculated as previously described¹⁴ using the expression of 48-genes previously shown to be down-regulated with MEK inhibition (set A) and 4 genes up-regulated with MEK inhibition (set B).⁸² In brief, expression data for set A and set B across the cohort (excluding MNG and HT) was log 2 transformed, and the Z score of the expression of each gene for each sample was calculated. For each sample, the Z-scores of set A genes were summed, and the Z-scores of set B genes were summed. The Z score sum of set B genes (up-regulated with MEK inhibition) was subtracted from the Z score sum of set A genes (down-regulated with MEK inhibition) to achieve a final ERK score for each sample.

PI3K-AKT-mTOR (PI3K) score calculation. The hallmark PI3K-AKT-mTOR signaling gene set⁸³ was used to calculate a PI3K activity score. Across our cohort of RNA-sequencing data (MNG and HT excluded), the expression data for each of the 105 genes in the score was log 2 transformed and Z-scores were calculated. For each sample, the activity score was calculated as the sum of the Z-scores for the 105 genes in the hallmark PI3K-AKT-mTOR signaling gene set.

MAP score calculation and enrichment analysis. MAP score was calculated from a list of 549 genes that were upregulated>4-fold with an adjusted p value of <0.05 in aggressive patient samples (relative to indolent) and >2-fold with an adjusted p value of <0.05 in BRAF-like (relative to RAS-like) patient samples. Across our cohort of RNA-sequencing data (MNG, FA, OA, HT excluded), the expression data for each of the 549 genes was log 2 transformed and Z-scores were calculated. For each sample, the MAP score was calculated as the average Z score for the 549 genes. Enrichment analysis of the 549 MAP score gene list was performed using a Panther overrepresentation test (pantherdb.org/).⁷²

### Example 17—Analysis of TCGA

Bulk RNA-sequencing, mutation, and clinical data from TCGA encompassing 496 PTCs14 was downloaded from cBioPortal.^{54,55} The clinical data included pre-calculated BRS and BRAF-like/RAS-like designations, disease free survival, and overall survival data. RNA gene-level expression values were downloaded from cBioPortal in RNA-Seq by Expectation Maximization (RSEM) and RSEM Z score formats.⁸⁴ MAP score was calculated for TCGA samples as the average Z score across 520 genes upregulated in BRAF-like and aggressive lesion samples in our cohort. Twenty-nine genes were excluded from the original 549 gene list because they were not covered in the TCGA sequencing data. TIMER 2.0 immune deconvolution data for TCGA samples, containing TIMER, CIBERSORT-Abs,⁷⁹ EPIC,⁷⁹ and MCPCOUNTER⁸⁰ algorithms, was downloaded from timer.cistrome.org.⁶⁷ For Tumor Immune Dysfunction Exclusion (TIDE) analysis,⁴⁷ RSEM formatted expression data were log 2 transformed and the log-fold change ratio was calculated for each gene in each sample. A log-fold change expression matrix was uploaded to TIDE for response prediction.

### Example 18—Multiplex Immunofluorescence (IF) of Formalin-Fixed Paraffin-Embedded (FFPE) Tissue

Data generation. Five μm ATC tissue sections were cut from 33 FFPE blocks and stored at −20° C. Tissue sections were thawed overnight at room temperature and heated for 1 h at 60° C. Tissue sections were deparaffinized with xylene (2× 15 min), ethanol (100% 2×5 minutes, 95% 1×5 minutes), and water (5 min) then washed with PBS. Antigen retrieval was performed by heating slides for 45 min in sodium citrate buffer (pH 6.0) in a rice cooker followed by 30 min at room temperature. Tissues were washed with PBS and blocked for 2 h with 10% goat serum in PBS (blocking buffer). Primary antibodies (Abcam ab207178 recombinant rabbit monoclonal anti-fibroblast activation protein alpha (FAP) IgG, clone EPR20021, 1:100; Invitrogen MA5-16868 rat monoclonal anti-MRC1 IgG2a, clone MR5D3, 1:25) were diluted in blocking buffer and incubated on tissue sections at 4° C. for 16 h (Abcam, Cambridge, UK; Thermo Fisher, Waltham, MA). Tissue sections were washed with 0.05% Tween 20 in PBS. Secondary antibodies (Invitrogen A-21245 polyclonal goat anti-rabbit IgG Alexa Fluor 647 1:150; Abcam ab6953 polyclonal goat anti-rat IgG Cy3 1:150) and conjugated primary antibodies (eBioscience 53-9003-82 mouse monoclonal anti-pan cytokeratin IgG1 AF488, clone AE1/AE3, 1:100) were diluted in blocking buffer containing Hoechst 33342 nuclear stain (1:1000) and incubated on tissue sections at 37° C. for 1 h (Abcam, Cambridge, UK; Thermo Fisher, Waltham, MA). 12 representative 20× and 12 representative 60×images were taken of each tissue section on a Nikon Spinning Disc confocal microscope.

Data analysis. Representative multiplex immunofluorescence images were scored by a practicing pathologist (VW). For each ATC tissue section, FAP staining of non-malignant cells were scored for intensity (0-3) and frequency (0-3). An overall FAP staining score was calculated as the product of the intensity and frequency scores (0-9). FAP staining scores of 0-1 were categorized as low. FAP staining scores of greater than 1 were categorized as high. The number of non-malignant MRC1 stained cells was counted on 12 20× images for each tissue section. An average of less than 1 MRC1+ cell per 20× field was categorized as low. Greater than 1 MRC1+ cell per 20× field was categorized as high.

### Example 19—Spatial Transcriptomics for FFPE

The Visium FFPE platform was used to generate spatial transcriptomics data (10× Genomics, Pleasonton, CA).

Slide preparation. 8 FFPE blocks of thyroid carcinomas with ATC histology were selected for Visium analysis. Following pathologist review (VW), 5 μm sections up to 6 mm×6 mm in size were cut onto a Visium Gene Expression Slide (Visium Spatial Gene Expression Slide Kit, PN-1000188). After sectioning, the slide was incubated at 42° C. and then stored in a desiccator until use.

Data generation. Following manufacturer's protocols (Visium FFPE 10× Genomics), samples were deparaffinized, stained (hematoxylin and eosin), and scanned at 20×. 3 of 8 ATCs were stained with hematoxylin only due to a supply chain shortage of eosin. The Visium Human Transcriptome Probe Set v1.0 was hybridized to samples overnight at 50° C. Following RNA digestion and tissue permeabilization, sequencing libraries were prepared per manufacturer's protocols. Sequencing was performed at a depth of >40,000 reads per spot, and >150 million reads per sample using the NovaSeq 6000 platform (Illumina, San Diego, CA).

Data analysis. Visium sequencing data was pre-processed with Space Ranger 2.0.0 (10× Genomics). Analysis of Space Ranger outputs was performed with Seurat 4.0.73 In brief, Seurat 4.0 was used to perform normalization, dimensionality reduction, and clustering. Dimensionality was determined by elbow plot. For clustering, resolution was set to 0.2. Designation of ATC histology was done by pathologist review (VW). Deconvolution of immune cell frequencies within individual capture areas was performed with the R package SpaCET.⁷⁴ For determining capture area malignant cell fraction, the SpaCET PANCAN setting was chosen. ATC classification was based on current standard-of-care clinical practice as outlined by the WHO and ATA guidelines and was reviewed by a practicing pathologist (VW). Individual Visium samples were determined to be MAP-high or MAP-moderate based on the MAP score of the associated bulk RNA sequencing sample.

### Example 20—Quantification and Statistical Analysis

Oncoplots were generated with the R packages maftools⁷⁵ and ComplexHeatmap.⁶⁹ Kaplan-Meier survival curves were compared and tested using the log rank test. PFS time and overall survival time were calculated as described in survival analyses methods above. Continuous outcomes were summarized by group with boxplots and tested using Wilcoxon rank-sum test. Kruskal-Wallis test with subsequent pairwise Wilcoxon rank-sum tests with Bonferroni's correction was used when comparing more than two groups. All statistical tests are two-sided unless otherwise specified. Logistic regression models were used to evaluate the association between aggressive disease and each predictive score. Penalized maximum likelihood with Jeffreys-prior penalty was used to allow for less biased and more stable estimation to account for the low number of events in some strata (R package brglm2).⁷⁶ Area under the receiver operating characteristic curve (AUC) and corresponding 95% confidence interval (CI) were computed to assess the discrimination ability of a fitted model. All statistical analyses were performed in R version 4.1.2 (R Foundation, Vienna, Austria).

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Lim, H., Devesa, S. S., Sosa, J. A., Check, D., and Kitahara, C. M. (2017). Trends in Thyroid Cancer Incidence and Mortality in the United States, 1974-2013. JAMA 317, 1338-1348. doi.org/10.1001/jama.2017.2719.
2. Rahib, L., Smith, B. D., Aizenberg, R., Rosenzweig, A. B., Fleshman, J. M., and Matrisian, L. M. (2014). Projecting cancer incidence and deaths to 2030: the unexpected burden of thyroid, liver, and pancreas cancers in the United States. Cancer Res. 74, 2913-2921. doi.org/10.1158/0008-5472.CAN-14-0155.
3. Haugen, B. R., Alexander, E. K., Bible, K. C., Doherty, G. M., Mandel, S. J., Nikiforov, Y. E., Pacini, F., Randolph, G. W., Sawka, A. M., Schlumberger, M., et al. (2015). American Thyroid Association Management Guidelines for Adult Patients with Thyroid Nodules and Differentiated Thyroid Cancer: The American Thyroid Association Guidelines Task Force on Thyroid Nodules and Differentiated Thyroid Cancer. Thyroid 26, 1-133. doi.org/10.1089/thy.2015.0020.
4. Filetti, S., Durante, C., Hartl, D., Leboulleux, S., Locati, L. D., Newbold, K., Papotti, M. G., and Berruti, A.; ESMO Guidelines Committee Electronic address clinicalguidelines@esmoorg (2019). Thyroid cancer: ESMO Clin¬ical Practice Guidelines for diagnosis, treatment and follow-up. Ann. On-col. 30, 1856-1883. doi.org/10.1093/annonc/mdz400.
5. Hwangbo, Y., Kim, J. M., Park, Y. J., Lee, E. K., Lee, Y. J., Park, D. J., Choi, Y. S., Lee, K. D., Sohn, S. Y., Kim, S. W., et al. (2017). Long-Term Recur¬rence of Small Papillary Thyroid Cancer and Its Risk Factors in a Korean Multicenter Study. J. Clin. Endocrinol. Metab. 102, 625-633. doi.org/10.1210/jc.2016-2287.
6. Nagaiah, G., Hossain, A., Mooney, C. J., Parmentier, J., and Remick, S. C. (2011). Anaplastic thyroid cancer: a review of epidemiology, pathogenesis, and treatment. JAMA Oncol. 2011. doi.org/10.1155/2011/542358.
7. Ali, S., and Cibas, E. (2018). The Bethesda System for Reporting Thyroid Cytopathology, 2 ed. (Springer International Publishing).
8. Hu, M. I., Waguespack, S. G., Dosiou, C., Ladenson, P. W., Livhits, M. J., Wirth, L. J., Sadow, P. M., Krane, J. F., Stack, B. C., Zafereo, M. E., et al. (2021). Afirma Genomic Sequencing Classifier and Xpression Atlas Molec¬ular Findings in Consecutive Bethesda III-VI Thyroid Nodules. J. Clin. Endo-crinol. Metab. 106, 2198-2207. doi.org/10.1210/clinem/dgab304.
9. Steward, D. L., Carty, S. E., Sippel, R. S., Yang, S. P., Sosa, J. A., Sipos, J. A., Figge, J. J., Mandel, S., Haugen, B. R., Burman, K. D., et al. (2019). Perfor¬mance of a Multigene Genomic Classifier in Thyroid Nodules With Indeter¬minate Cytology: A Prospective Blinded Multicenter Study. JAMA Oncol. 5, 204-212. doi.org/10.1001/jamaoncol.2018.4616.
10. Cohen, Y., Xing, M., Mambo, E., Guo, Z., Wu, G., Trink, B., Beller, U., Westra, W. H., Ladenson, P. W., and Sidransky, D. (2003). BRAF mutation in papillary thyroid carcinoma. J. Natl. Cancer Inst. 95, 625-627. doi.org/10.1093/jnci/95.8.625.
11. Nikiforov, Y. E., and Nikiforova, M. N. (2011). Molecular genetics and diag¬nosis of thyroid cancer. Nat. Rev. Endocrinol. 7, 569-580. doi.org/10.1038/nrendo.2011.142.
12. Xing, M. (2013). Molecular pathogenesis and mechanisms of thyroid can¬cer. Nat. Rev. Cancer 13, 184-199. doi.org/10.1038/nrc3431.
13. Lemoine, N. R., Mayall, E. S., Wyllie, F. S., Farr, C. J., Hughes, D., Padua, R. A., Thurston, V., Williams, E. D., and Wynford-Thomas, D. (1988). Acti¬vated ras oncogenes in human thyroid cancers. Cancer Res. 48, 4459-4463.
14. Cancer Genome Atlas Research Network (2014). Integrated genomic characterization of papillary thyroid carcinoma. Cell 159, 676-690. doi.org/10.1016/j.cell.2014.09.050.
15. Nikiforova, M. N., Mercurio, S., Wald, A. I., Barbi de Moura, M., Callenberg, K., Santana-Santos, L., Gooding, W. E., Yip, L., Ferris, R. L., and Nikiforov, Y. E. (2018). Analytical performance of the ThyroSeq v3 genomic classifier for cancer diagnosis in thyroid nodules. Cancer 124, 1682-1690. doi.org/10.1002/cncr.31245.
16. Nikiforova, M. N., Tseng, G. C., Steward, D., Diorio, D., and Nikiforov, Y. E. (2008). MicroRNA expression profiling of thyroid tumors: biological signif¬icance and diagnostic utility. J. Clin. Endocrinol. Metab. 93, 1600-1608. doi.org/10.1210/jc.2007-2696.
17. Gopal, R. K., Ku€bler, K., Calvo, S. E., Polak, P., Livitz, D., Rosebrock, D., Sadow, P. M., Campbell, B., Donovan, S. E., Amin, S., et al. (2018). Wide¬spread Chromosomal Losses and Mitochondrial DNA Alterations as Genetic Drivers in Huerthle Cell Carcinoma. Cancer Cell 34, 242-255.e5. doi.org/10.1016/j.ccell.2018.06.013.
18. Sicklick, J. K., Kato, S., Okamura, R., Schwaederle, M., Hahn, M. E., Wil¬liams, C. B., De, P., Krie, A., Piccioni, D. E., Miller, V. A., et al. (2019). Mole¬cular profiling of cancer patients enables personalized combination ther¬apy: the I-PREDICT study. Nat. Med. 25, 744-750. doi.org/10. 1038/s41591-019-0407-5.
19. Hays, P. (2021). Translational Personalized Medicine: Molecular Profiling, Druggable Targets, and Clinical Genomic Medicine (Springer).
20. Xing, M., Alzahrani, A. S., Carson, K. A., Viola, D., Elisei, R., Bendlova, B., Yip, L., Mian, C., Vianello, F., Tuttle, R. M., et al. (2013). Association be¬tween BRAF V600E mutation and mortality in patients with papillary thyroid cancer. JAMA 309, 1493-1501. doi.org/10.1001/jama.2013.3190.
21. Landa, I., Ibrahimpasic, T., Boucai, L., Sinha, R., Knauf, J. A., Shah, R. H., Dogan, S., Ricarte-Filho, J. C., Krishnamoorthy, G. P., Xu, B., et al. (2016). Genomic and transcriptomic hallmarks of poorly differentiated and anaplastic thyroid cancers. J. Clin. Invest. 126, 1052-1066. doi.org/10.1172/JCI85271.
22. Pozdeyev, N., Gay, L. M., Sokol, E. S., Hartmaier, R., Deaver, K. E., Davis, S., French, J. D., Borre, P. V., LaBarbera, D. V., Tan, A. C., et al. (2018). Ge¬netic Analysis of 779 Advanced Differentiated and Anaplastic Thyroid Cancers. Clin. Can¬cer Res. 24, 3059-3068. doi.org/10.1158/1078-0432.CCR-18-0373.
23. Xu, B., Fuchs, T., Dogan, S., Landa, I., Katabi, N., Fagin, J. A., Tuttle, R. M., Sherman, E., Gill, A. J., and Ghossein, R. (2020). Dissecting Anaplastic Thyroid Carcinoma: A Comprehensive Clinical, Histologic, Immunopheno-typic, and Molecular Study of 360 Cases. Thyroid 30, 1505-1517. doi.org/10.1089/thy.2020.0086.
24. Yoo, S. K., Song, Y. S., Lee, E. K., Hwang, J., Kim, H. H., Jung, G., Kim, Y. A., Kim, S. J., Cho, S. W., Won, J. K., et al. (2019). Integrative analysis of genomic and transcriptomic characteristics associated with progression of aggressive thyroid cancer. Nat. Commun. 10, 2764. doi.org/10.1038/s41467-019-10680-5.
25. Mady, L. J., Grimes, M. C., Khan, N. I., Rao, R. H., Chiosea, S. I., Yip, L., Fer¬ris, R. L., Nikiforov, Y. E., Carty, S. E., and Duvvuri, U. (2020). Molecular Pro¬file of Locally Aggressive Well Differentiated Thyroid Cancers. Sci. Rep. 10, 8031. doi.org/10.1038/s41598-020-64635-8.
26. Jin, M., Song, D. E., Ahn, J., Song, E., Lee, Y. M., Sung, T. Y., Kim, T. Y., Kim, W. B., Shong, Y. K., Jeon, M. J., and Kim, W. G. (2021). Genetic Profiles of Aggressive Variants of Papillary Thyroid Carcinomas. Cancers 13, 892. doi.org/10.3390/cancers13040892.
27. Yip, L., Gooding, W. E., Nikitski, A., Wald, A. I., Carty, S. E., Karslioglu-French, E., Seethala, R. R., Zandberg, D. P., Ferris, R. L., Nikiforova, M. N., and Nikiforov, Y. E. (2021). Risk assessment for distant metastasis in differ¬entiated thyroid cancer using molecular profiling: A matched case-control study. Cancer 127, 1779-1787. doi.org/10.1002/cncr.33421.
28. Xiao, Y., and Yu, D. (2021). Tumor microenvironment as a therapeutic target in cancer. Pharmacol. Ther. 221, 107753. doi.org/10.1016/j.pharmthera.2020.107753.
29. Pu, W., Shi, X., Yu, P., Zhang, M., Liu, Z., Tan, L., Han, P., Wang, Y., Ji, D., Gan, H., et al. (2021). Single-cell transcriptomic analysis of the tumor eco¬systems underlying initiation and progression of papillary thyroid carcinoma. Nat. Commun. 12, 6058. doi.org/10.1038/s41467-021-26343-3.
30. Jolly, L. A., Novitskiy, S., Owens, P., Massoll, N., Cheng, N., Fang, W., Moses, H. L., and Franco, A. T. (2016). Fibroblast-Mediated Collagen Re¬modeling Within the Tumor Microenvironment Facilitates Progression of Thyroid Cancers Driven by BrafV600E and Pten Loss. Cancer Res. 76, 1804-1813. doi.org/10.1158/0008-5472.CAN-15-2351.
31. Fang, W., Ye, L., Shen, L., Cai, J., Huang, F., Wei, Q., Fei, X., Chen, X., Guan, H., Wang, W., et al. (2014). Tumor-associated macrophages pro¬mote the metastatic potential of thyroid papillary cancer by releasing CXCL8. Carcinogenesis 35, 1780-1787. doi.org/10.1093/carcin/bgu060.
32. Lu, L., Wang, J. R., Henderson, Y. C., Bai, S., Yang, J., Hu, M., Shiau, C. K., Pan, T., Yan, Y., Tran, T. M., et al. (2023). Anaplastic transformation in thy¬roid cancer revealed by single cell transcriptomics. J. Clin. Invest. 133, e169653. doi.org/10.1172/JCI169653.
33. Dierks, C., Seufert, J., Aumann, K., Ruf, J., Klein, C., Kiefer, S., Rassner, M., Boerries, M., Zielke, A., la Rosee, P., et al. (2021). Combination of Len-vatinib and Pembrolizumab Is an Effective Treatment Option for Anaplastic and Poorly Differentiated Thyroid Carcinoma. Thyroid 31, 1076-1085. doi.org/10.1089/thy.2020.0322.
34. Dierks, C., Ruf, J., Seufert, J., Kreissl, M., Klein, C., Spitzweg, C., Kroiss, M., Thomusch, O., Lorenz, K., Zielke, A., and Miething, C. (2022). 1646MO Phase II ATLEP trial: Final results for lenvatinib/pembrolizumab in metas¬tasized anaplastic and poorly differentiated thyroid carcinoma. Ann. On-col. 33, S1295.
35. Study of Cemiplimab Combined with Dabrafenib and Trametinib in People with Anaplastic Thyroid Cancer. ClinicalTrials.gov/show/NCT04238624.
36. Pembrolizumab, D. Trametinib before Surgery for the Treatment of BRAF-Mutated Anaplastic Thyroid Cancer. ClinicalTrials.gov/show/NCT04675710.
37. Lenvatinib and Pembrolizumab for the Treatment of Stage IVB Locally Advanced and Unresectable or Stage IVC Metastatic Anaplastic Thyroid Cancer. ClinicalTrials.gov/show/NCT04171622.
38. Atezolizumab with Chemotherapy in Treating Patients with Anaplastic or Poorly Differentiated Thyroid Cancer. ClinicalTrials.gov/show/NCT03181100.
39. Manzella, L., Stella, S., Pennisi, M. S., Tirrô, E., Massimino, M., Romano, C., Puma, A., Tavarelli, M., and Vigneri, P. (2017). New Insights in Thyroid Cancer and p53 Family Proteins. Int. J. Mol. Sci. 18, 1325. doi.org/10.3390/ijms18061325.
40. Lai, W. A., Liu, C. Y., Lin, S. Y., Chen, C. C., and Hang, J. F. (2020). Charac¬terization of Driver Mutations in Anaplastic Thyroid Carcinoma Identifies. Cancers 12, 1973. doi.org/10.3390/cancers12071973.
41. Landa, I., Ganly, I., Chan, T. A., Mitsutake, N., Matsuse, M., Ibrahimpasic, T., Ghossein, R. A., and Fagin, J. A. (2013). Frequent somatic TERT pro¬moter mutations in thyroid cancer: higher prevalence in advanced forms of the disease. J. Clin. Endocrinol. Metab. 98, E1562-E1566. doi.org/10.1210/jc.2013-2383.
42. Hiltzik, D., Carlson, D. L., Tuttle, R. M., Chuai, S., Ishill, N., Shaha, A., Shah, J. P., Singh, B., and Ghossein, R. A. (2006). Poorly differentiated thyroid carcinomas defined on the basis of mitosis and necrosis: a clinicopatho-logic study of 58 patients. Cancer 106, 1286-1295. doi.org/10. 1002/cncr.21739.
43. Volante, M., Collini, P., Nikiforov, Y. E., Sakamoto, A., Kakudo, K., Katoh, R., Lloyd, R. V., LiVolsi, V. A., Papotti, M., Sobrinho-Simoes, M., et al. (2007). Poorly differentiated thyroid carcinoma: the Turin proposal for the use of uniform diagnostic criteria and an algorithmic diagnostic approach. Am. J. Surg. Pathol. 31, 1256-1264. doi.org/10.1097/PAS.0b013e3180309e6a.
44. Ragazzi, M., Ciarrocchi, A., Sancisi, V., Gandolfi, G., Bisagni, A., and Piana, S. (2014). Update on anaplastic thyroid carcinoma: morphological, molecular, and genetic features of the most aggressive thyroid cancer. Internet J. Endo-crinol. 2014, 790834. doi.org/10.1155/2014/790834.
45. Biffi, G., and Tuveson, D. A. (2021). Diversity and Biology of Cancer-Associated Fibroblasts. Physiol. Rev. 101, 147-176. doi.org/10. 1152/physrev.00048.2019.
46. Giannini, R., Moretti, S., Ugolini, C., Macerola, E., Menicali, E., Nucci, N., Morelli, S., Colella, R., Mandarano, M., Sidoni, A., et al. (2019). Immune Profiling of Thyroid Carcinomas Suggests the Existence of Two Major Phe¬notypes: An ATC-Like and a PDTC-Like. J. Clin. Endocrinol. Metab. 104, 3557-3575. doi.org/10.1210/jc.2018-01167.
47. Jiang, P., Gu, S., Pan, D., Fu, J., Sahu, A., Hu, X., Li, Z., Traugh, N., Bu, X., Li, B., et al. (2018). Signatures of T cell dysfunction and exclusion predict cancer immunotherapy response. Nat. Med. 24, 1550-1558. doi.org/10.1038/s41591-018-0136-1.
48. Mao, X., Xu, J., Wang, W., Liang, C., Hua, J., Liu, J., Zhang, B., Meng, Q., Yu, X., and Shi, S. (2021). Crosstalk between cancer-associated fibro¬blasts and immune cells in the tumor microenvironment: new findings and future perspectives. Mol. Cancer 20, 131. doi.org/10.1186/s12943-021-01428-1.
49. Lek, M., Karczewski, K. J., Minikel, E. V., Samocha, K. E., Banks, E., Fen¬nell, T., O'Donnell-Luria, A. H., Ware, J. S., Hill, A. J., Cummings, B. B., et al. (2016). Analysis of protein-coding genetic variation in 60,706 hu¬mans. Nature 536, 285-291. doi.org/10.1038/nature19057.
50. Karczewski, K. J., Francioli, L. C., Tiao, G., Cummings, B. B., Alföldi, J., Wang, Q., Collins, R. L., Laricchia, K. M., Ganna, A., Birnbaum, D. P., et al. (2020). The mutational constraint spectrum quantified from variation in 141,456 humans. Nature 581, 434-443. doi.org/10.1038/s41586-020-2308-7.
51. Frankish, A., Diekhans, M., Jungreis, I., Lagarde, J., Loveland, J. E., Mudge, J. M., Sisu, C., Wright, J. C., Armstrong, J., Barnes, I., et al. (2021). GENCODE 2021. Nucleic Acids Res. 49, D916-D923. doi.org/10.1093/nar/gkaa1087.
52. Ashburner, M., Ball, C. A., Blake, J. A., Botstein, D., Butler, H., Cherry, J. M., Davis, A. P., Dolinski, K., Dwight, S. S., Eppig, J. T., et al. (2000). Gene ontology: tool for the unification of biology. The Gene Ontology Consortium. Nat. Genet. 25, 25-29. doi.org/10.1038/75556.
53. Aleksander, S. A., Balhoff, J., Carbon, S., Cherry, J. M., Drabkin, H. J., Ebert, D., Feuermann, M., Gaudet, P., Harris, N. L., Hill, D. P., et al. (2023). The Gene Ontology knowledgebase in 2023. Genetics 224. doi.org/10.1093/genetics/iyad031.
54. Gao, J., Aksoy, B. A., Dogrusoz, U., Dresdner, G., Gross, B., Sumer, S. O., Sun, Y., Jacobsen, A., Sinha, R., Larsson, E., et al. (2013). Integrative anal¬ysis of complex cancer genomics and clinical profiles using the cBioPortal. Sci. Signal. 6, p11. doi.org/10.1126/scisignal.2004088.
55. Cerami, E., Gao, J., Dogrusoz, U., Gross, B. E., Sumer, S. O., Aksoy, B. A., Jacobsen, A., Byrne, C. J., Heuer, M. L., Larsson, E., et al. (2012). The cBio cancer genomics portal: an open platform for exploring multidimensional cancer genomics data. Cancer Discov. 2, 401-404. doi.org/10. 1158/2159-8290.CD-12-0095.
56. Koelsche, C., Renner, M., Hartmann, W., Brandt, R., Lehner, B., Waldbur-ger, N., Alldinger, I., Schmitt, T., Egerer, G., Penzel, R., et al. (2014). TERT promoter hotspot mutations are recurrent in myxoid liposarcomas but rare in other soft tissue sarcoma entities. J. Exp. Clin. Cancer Res. 33, 33. doi.org/10.1186/1756-9966-33-33.
57. Martin, M. (2011). Cutadapt Removes Adapter Sequences from High-Throughput Sequencing Reads (EMBnet).
58. Andrews, S. (2010). FastQC: A Quality Control Tool for High Throughput Sequence Data.
59. Li, H., and Durbin, R. (2010). Fast and accurate long-read alignment with Burrows-Wheeler transform. Bioinformatics 26, 589-595. doi.org/10.1093/bioinformatics/btp698.
60. McKenna, A., Hanna, M., Banks, E., Sivachenko, A., Cibulskis, K., Kernyt-sky, A., Garimella, K., Altshuler, D., Gabriel, S., Daly, M., and DePristo, M. A. (2010). The Genome Analysis Toolkit: a MapReduce framework for analyzing next-generation DNA sequencing data. Genome Res. 20, 1297-1303. doi.org/10.1101/gr.107524.110.
61. Dobin, A., Davis, C. A., Schlesinger, F., Drenkow, J., Zaleski, C., Jha, S., Batut, P., Chaisson, M., and Gingeras, T. R. (2013). STAR: ultrafast univer¬sal RNA-seq aligner. Bioinformatics 29, 15-21. doi.org/10.1093/bioinformatics/bts635.
62. Liao, Y., Smyth, G. K., and Shi, W. (2014). featureCounts: an efficient gen¬eral purpose program for assigning sequence reads to genomic features. Bioinformatics 30, 923-930. doi.org/10.1093/bioinformatics/btt656.
63. Love, M. I., Huber, W., and Anders, S. (2014). Moderated estimation of fold change and dispersion for RNA-seq data with DESeq2. Genome Biol. 15, 550. doi.org/10.1186/s13059-014-0550-8.
64. Blighe, K., Rana, S., and Lewis, M. (2018). EnhancedVolcano: publication-ready volcano plots with enhanced colouring and labeling. bioconductor.org/packages/devel/bioc/vignettes/EnhancedVolcano/inst/doc/EnhancedVolcano.html.
65. Zhao, S., Guo, Y., Sheng, Q., and Shyr, Y. (2014). Heatmap3: an improved heatmap package with more powerful and convenient features. BMC Bio-inf. 15. doi.org/10.1186/1471-2105-15-S10-P16.
66. Subramanian, A., Tamayo, P., Mootha, V. K., Mukherjee, S., Ebert, B. L., Gillette, M. A., Paulovich, A., Pomeroy, S. L., Golub, T. R., Lander, E. S., and Mesirov, J. P. (2005). Gene set enrichment analysis: a knowledge-based approach for interpreting genome-wide expression profiles. Proc. Natl. Acad. Sci. USA 102, 15545-15550. doi.org/10.1073/pnas. 0506580102.
67. Li, T., Fu, J., Zeng, Z., Cohen, D., Li, J., Chen, Q., Li, B., and Liu, X. S. (2020). TIMER2.0 for analysis of tumor-infiltrating immune cells. Nucleic Acids Res. 48, W509-W514. doi.org/10.1093/nar/gkaa407.
68. Wickham, H. (2016). ggplot2: Elegant Graphics for Data Analysis (Springer). ggplot2.tidyverse.org.
69. Gu, Z. (2022). Complex Heatmap Visualization 1, e43.
70. Hass, B., Dobin, A., Stransky, N., Li, B., Yang, X., Tickle, T., Bankapur, A., Ganote, C., Doak, T., and Pochet, N. (2017). STAR-Fusion: fast and accu¬rate fusion transcript detection from RNA-seq. Preprint at bioRxiv. doi.org/10.1101/120295.
71. Robinson, J. T., Thorvaldsdóttir, H., Winckler, W., Guttman, M., Lander, E. S., Getz, G., and Mesirov, J. P. (2011). Integrative genomics viewer. Nat. Biotechnol. 29, 24-26. doi.org/10.1038/nbt.1754.
72. Thomas, P. D., Ebert, D., Muruganujan, A., Mushayahama, T., Albou, L. P., and Mi, H. (2022). PANTHER: Making genome-scale phylogenetics acces¬sible to all. Protein Sci. 31, 8-22. doi.org/10.1002/pro.4218.
73. Hao, Y., Hao, S., Andersen-Nissen, E., Mauck, W. M., Zheng, S., Butler, A., Lee, M. J., Wilk, A. J., Darby, C., Zager, M., et al. (2021). Integrated analysis of multimodal single-cell data. Cell 184, 3573-3587.e29. doi.org/10.1016/j.cell.2021.04.048.
74. Ru, B., Huang, J., Zhang, Y., Aldape, K., and Jiang, P. (2023). Estimation of cell lineages in tumors from spatial transcriptomics data. Nat. Commun. 14, 568. doi.org/10.1038/s41467-023-36062-6.
75. Mayakonda, A., Lin, D. C., Assenov, Y., Plass, C., and Koeffler, H. P. (2018). Maftools: efficient and comprehensive analysis of somatic variants in cancer. Genome Res. 28, 1747-1756. doi.org/10.1101/gr.239244.118.
76. Kosmidis, I., Kenne Pagui, E. C., Konis, K., and Sartori, N. (2023). brglm2: Bias Reduction in Generalized Linear Models. CRAN.R-project. org/package=brglm2.
77. Wang, K., Li, M., and Hakonarson, H. (2010). ANNOVAR: functional anno¬tation of genetic variants from high-throughput sequencing data. Nucleic Acids Res. 38, e164. doi.org/10.1093/nar/gkq603.
78. Li, M. M., Datto, M., Duncavage, E. J., Kulkarni, S., Lindeman, N. I., Roy, S., Tsimberidou, A. M., Vnencak-Jones, C. L., Wolff, D. J., Younes, A., and Ni-kiforova, M. N. (2017). Standards and Guidelines for the Interpretation and Reporting of Sequence Variants in Cancer: A Joint Consensus Recom¬mendation of the Association for Molecular Pathology, American Society of Clinical Oncology, and College of American Pathologists. J. Mol. Diagn. 19, 4-23. doi.org/10.1016/j.jmoldx.2016.10.002.
79. Newman, A. M., Liu, C. L., Green, M. R., Gentles, A. J., Feng, W., Xu, Y., Hoang, C. D., Diehn, M., and Alizadeh, A. A. (2015). Robust enumeration of cell subsets from tissue expression profiles. Nat. Methods 12, 453-457. doi.org/10.1038/nmeth.3337.
80. Becht, E., Giraldo, N. A., Lacroix, L., Buttard, B., Elarouci, N., Petitprez, F., Selves, J., Laurent-Puig, P., Saute's-Fridman, C., Fridman, W. H., and de Reynie's, A. (2016). Estimating the population abundance of tissue-infil¬trating immune and stromal cell populations using gene expression. Genome Biol. 17, 218. doi.org/10.1186/s13059-016-1070-5.
81. Stransky, N., Cerami, E., Schalm, S., Kim, J. L., and Lengauer, C. (2014). The landscape of kinase fusions in cancer. Nat. Commun. 5, 4846. doi.org/10.1038/ncomms5846.
82. Pratilas, C. A., Taylor, B. S., Ye, Q., Viale, A., Sander, C., Solit, D. B., and Rosen, N. (2009). (V600E) BRAF is associated with disabled feedback inhi¬bition of RAF-MEK signaling and elevated transcriptional output of the pathway. Proc. Natl. Acad. Sci. USA 106, 4519-4524. doi.org/10. 1073/pnas.0900780106.
83. Liberzon, A., Birger, C., Thorvaldsdóttir, H., Ghandi, M., Mesirov, J. P., and Tamayo, P. (2015). The Molecular Signatures Database (MSigDB) hallmark gene set collection. Cell Syst. 1, 417-425. doi.org/10.1016/j.cels. 2015.12.004.
84. Li, B., and Dewey, C. N. (2011). RSEM: accurate transcript quantification from RNA-Seq data with or without a reference genome. BMC Bioinf. 12, 323. doi.org/10.1186/1471-2105-12-323.
85. Xu G J, Loberg M A, Gallant J N, Sheng Q, Chen S C, Lehmann B D, Shaddy S M, Tigue M L, Phifer C J, Wang L, Saab-Chalhoub M W, Dehan L M, Wei Q, Chen R, Li B, Kim C Y, Ferguson D C, Netterville J L, Rohde S L, Solórzano C C, Bischoff L A, Baregamian N, Shaver A C, Mehrad M, Ely K A, Byrne D W, Stricker T P, Murphy B A, Choe J H, Kagohara L T, Jaffee E M, Huang E C, Ye F, Lee E, Weiss V L. Molecular signature incorporating the immune microenvironment enhances thyroid cancer outcome prediction. Cell Genom. 2023 Sep. 14; 3 (10): 100409. doi: 10.1016/j.xgen.2023.100409.
86. Gao, Y, Li, J., Cheng, W., Diao, T., Liu, H., Bo, Y., Liu, C., Zhou, W., Chen, M., Zhang, Y., Liu, Z., Han, W., Chen, R., Peng, J., Zhu, L., Hou, W., Zhang, Z. Cross-Tissue human fibroblast atlas reveals myofibroblast subtypes with distinct roles in immune modulation. Cancer Cell. 2024 Oct. 14; 42 (1): P1764-1783.
87. Kieffer Y, Hocine H R, Gentric G, Pelon F, Bernard C, Bourachot B, Lameiras S, Albergante L, Bonneau C, Guyard A, Tarte K, Zinovyev A, Baulande S, Zalcman G, Vincent-Salomon A, Mechta-Grigoriou F. Single-Cell Analysis Reveals Fibroblast Clusters Linked to Immunotherapy Resistance in Cancer. Cancer Discov. 2020 September; 10 (9): 1330-1351. doi: 10.1158/2159-8290.
88. Costa A, Kieffer Y, Scholer-Dahirel A, Pelon F, Bourachot B, Cardon M, Sirven P, Magagna I, Fuhrmann L, Bernard C, Bonneau C, Kondratova M, Kuperstein I, Zinovyev A, Givel A M, Parrini M C, Soumelis V, Vincent-Salomon A, Mechta-Grigoriou F. Fibroblast Heterogeneity and Immunosuppressive Environment in Human Breast Cancer. Cancer Cell. 2018 Mar. 12; 33 (3): 463-479.e10. doi: 10.1016/j.ccell.2018.01.011.
89. Fornage, B. D. (2020). Errors and Pitfalls in Ultrasound-Guided Fine-Needle Aspiration. In: Interventional Ultrasound of the Breast. Springer, Cham. doi.org/10.1007/978-3-030-20829-5.
90. Hijioka S, Hara K, Mizuno N, Imaoka H, Bhatia V, Mekky M A, Yoshimura K, Yoshida T, Okuno N, Hieda N, Tajika M, Tanaka T, Ishihara M, Yatabe Y, Shimizu Y, Niwa Y, Yamao K. Diagnostic performance and factors influencing the accuracy of EUS-FNA of pancreatic neuroendocrine neoplasms. J Gastroenterol. 2016 September; 51 (9): 923-30. doi: 10.1007/s00535-016-1164-6.
91. Kundu U, Gan Q, Donthi D, Sneige N. The Utility of Fine Needle Aspiration (FNA) Biopsy in the Diagnosis of Mediastinal Lesions. Diagnostics (Basel). 2023 Jul. 18; 13 (14): 2400. doi: 10.3390/diagnostics13142400.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for identifying enrichment of a fibroblast subtype in a thyroid cancer, comprising:
obtaining biopsy sample from a subject, the sample including nucleic acids;
assaying the nucleic acids in the sample for an increase in the amount of mRNA produced by genes consisting of 3-80 of the genes set forth in Table 1A, Table 1B, Table 1C, Table 1D, Table 1E, Table 2A, Table 2B, Table 2C, Table 2D, Table 2E, Table 2F, Table 3, Table 4, and Table 5 relative to a control level of mRNA produced by the genes in a sample obtained from a healthy subject; and
identifying an enrichment of the fibroblast subset population if there is an increase in the amount of mRNA in the sample relative to the control level.

2. The method of claim 1, wherein the biopsy sample is a fine needle aspiration (FNA) biopsy sample.

3. The method of claim 2, wherein a portion of the genetic material in the FNA biopsy sample is from fibroblasts.

4. The method of claim 1, wherein the subject is being evaluated for presence, metastasis, and/or recurrence of thyroid cancer.

5. The method of claim 1, wherein biopsy sample is taken from a tumor microenvironment.

6. A method for identifying a fibroblast subtype, macrophage subtype, or myofibroblast subtypes in a thyroid cancer, comprising:
obtaining biopsy sample from a subject, the sample including nucleic acids;
assaying the nucleic acids in the sample for an increase in the amount of mRNA produced by genes consisting of 3-80 of the genes set forth in Table 1A, Table 1B, Table 1C, Table 1D, Table 1E, Table 2A, Table 2B, Table 2C, Table 2D, Table 2E, Table 2F, Table 3, Table 4, and Table 5 relative to a control level of mRNA produced by the genes in a sample obtained from a healthy subject; and
and
identifying an enrichment of fibroblast subtype, macrophage subtype, or myofibroblast subtypes if there is an increase in the amount of mRNA in the sample relative to the control level.

7. The method of claim 6, wherein the biopsy sample is a fine needle aspiration (FNA) biopsy sample.

8. The method of claim 7, wherein a portion of the genetic material in the FNA biopsy sample is from fibroblasts.

9. The method of claim 6, wherein the subject is being evaluated for presence, metastasis, and/or recurrence of thyroid cancer.

10. The method of claim 6, wherein biopsy sample is taken from a tumor microenvironment.

11. A method of detecting expression of genes in a fine needle aspiration (FNA) biopsy sample from a subject, comprising the steps of:
determining expression levels in the FNA biopsy sample of each of the gene of a fibroblast gene signature, wherein the fibroblast gene signature comprises 3-80 of the genes set forth in Table 1A, Table 1B, Table 1C, Table 1D, Table 1E, Table 2A, Table 2B, Table 2C, Table 2D, Table 2E, Table 2F, Table 3, Table 4, and Table 5;
wherein the expression levels of each of the genes are determined by sequencing genetic material in the FNA biopsy sample.

12. The method of claim 11, wherein a portion of the genetic material in the FNA biopsy sample is from fibroblasts.

13. The method of claim 11, wherein the subject is being evaluated for presence, metastasis, and/or recurrence of thyroid cancer.

14. The method of claim 11, wherein biopsy sample is taken from a tumor microenvironment.

15. The method of claim 13, and further comprising comparing the expression levels to expression levels for each gene in a control sample from a normal healthy individual or a benign individual, wherein overexpression of the genes in the FNA biopsy sample relative to the control is associated with high risk of presence, metastasis, and/or recurrence of thyroid cancer.

16. The method of claim 15, and further comprising administering radioactive iodine to the subject when there is overexpression of the genes in the FNA biopsy sample relative expression levels of the genes in a control sample from a normal healthy individual or a benign individual.
